(19) United States
(12) Patent Application Publication
(10) Pub. No.: US 2023/0242473 A1
(43) Pub. Date: Aug. 3, 2023

(54) THYROMIMETICS

(71) Applicant: Autobahn Therapeutics, Inc., San Diego, CA (US)

(72) Inventors: Thomas von GELDERN, Richmond, IL (US); Bradley BACKES, San Francisco, CA (US)

(21) Appl. No.: 18/002,036

(22) PCT Filed: Jun. 17, 2021
(86) PCT No.: PCT/US21/37833

§ 371 Date: Dec. 15, 2022

Related U.S. Application Data
(60) Provisional application No. 63/040,448, filed on Jun. 17, 2020.
(60) Provisional application No. 63/040,452, filed on Jun. 17, 2020.

(51) Int. Cl. C07C 69/618; C07C 59/72; C07C 235/20; C07C 59/70

(52) U.S. Cl. CPC C07C 69/618; C07C 59/72; C07C 235/20; C07C 59/70; C07C 2602/08

(57) ABSTRACT

Compounds are provided herein function as thyromimetics and have utility for treating diseases such as neurodegenerative disorders and fibrotic diseases. Pharmaceutical compositions containing such compounds are also provided, as are methods of their preparation.

### CROSS-REFERENCE

This application claims benefit of U.S. Provisional Patent Application No. 63/040,448, filed on Jun. 17, 2020 and U.S. Provisional Patent Application No. 63/040,452, filed on Jun. 17, 2020, which are both incorporated herein by reference in their entirety.

### BACKGROUND

### Technical Field

The invention relates to thyromimetic compounds and to products containing the same, as well as to methods of their use and preparation.

### Description of the Related Art

Thyroid hormone (TH) is a key signal for oligodendrocyte differentiation and myelin formation during development, and also stimulates remyelination in adult models of multiple sclerosis (MS) (Calzà et al., Brain Res Revs 48:339-346, 2005). However, TH is not an acceptable long-term therapy due to the limited therapeutic window in which remyelination can be achieved while avoiding the cardiotoxicity and bone demineralization associated with chronic hyperthyroidism. Some thyroid hormone analogs can activate thyroid hormone-responsive genes while avoiding the associated downsides of TH by exploiting molecular and physiological features of thyroid hormone receptors (Malm et al., Mini Rev Med Chem 7:79-86, 2007). These receptors are expressed in two major forms with heterogenous tissue distributions and overlapping but distinct sets of target genes (Yen, Physiol Rev 81:1097-1142, 2001). TRα is enriched in the heart, brain, and bone while TRβ is enriched in the liver (O'Shea et al., Nucl ReceptSignal 4:e011, 2006).

It has also been reported that TH can inhibit the transforming growth factor beta (TGF-β) signaling, and, therefore, attenuate fibrotic responses (Alonso-Merino et al., Proc Natl Acad Sci USA. 113(24):E3451-60, 2016). TGF-β is a cytokine with pleiotropic effects in tissue homeostasis that plays a key role in pathological processes such as fibrosis (Massagué, Nat Rev Mol Cell Biol. 13(10):616-630, 2012). By inhibiting TGF-β signalling, TR ligands or agonists could have beneficial effects to block the progression of fibrotic diseases, such as idiopathic pulmonary fibrosis (IPF) or systemic sclerosis (Varga et al., Curr Opin Rheumatol. 20(6): 720-728, 2008).

Developing selective thyromimetics has been challenging due to the high sequence homology of thyroid hormone receptor subtypes; namely, only one amino acid residue on the internal surface of the ligand binding domain cavity varies between the α1 and β1 forms. Despite this challenge, several groups have reported TRβ-selective agonists. Scanlan et al. identified GC-1 (sobetirome) as one of the first potent analogs to demonstrate significant TRβ-selectivity in vitro (Chiellini et al., Chem Biol 5:299-306, 1998; Yoshihara et al., J Med Chem 46:3152-3161, 2003) and in vivo (Trost et al., Endocrinology 141:3057-3064, 2000; Grover et al., Endocrinology 145:1656-1661, 2004; Baxter et al., Trends Endocrinol Metab 15:154-157, 2004). As used herein, the term “sobetirome” refers to a synthetic diarylmethane derivative that was investigated clinically as a potential therapeutic for hypercholesterolemia (see U.S. Pat. No. 5,883,294, which is incorporated by reference herein). Other names for sobetirome found in the literature and regulatory filings include QRX-431 and GC-1. Metabasis employs a similar core with a novel liver-targeting prodrug strategy in MB07811 (Erion et al., PNAS 104(39), 15490-15495, 2007). Madrigal has reported TRβ-selective activity in vivo for MGL-3196 (Taub et al., Atherosclerosis 230(2):373-380, 2013). KaroBio has reported on eprotirome (KB2115; Berkenstam et al., PNAS 105(2):663-668, 2008) and KB-141 (Ye et al., J Med Chem 46:1580-1588, 2003), both of which demonstrate improved TRβ-selectivity in vitro. Further studies from this group highlight additional selective compounds (Hangeland et al., BMCL 14:3549-3553, 2004). Two TRβ-selective agonists, identified as SKL-12846 and SKL-13784, have been reported to accumulate in the liver and to reduce cholesterol levels in rodents (Takahashi et al., BMC 22(1):488-498, 2014; Xenobiotica 2015, 1-9). Kissei has also reported selective compounds (Shiohara et al., BMC 20(11), 3622-3634, 2012).

While progress has been made in this field, there remains a need in the art for further selective thyromimetic compounds, as well as to products containing the same, and for methods related to their use and preparation. It is the consensus view from the thyromimetic literature that 3,5 inner ring di-substitution is required for potency in T3 analogs, with dramatic losses in potency observed when one of these inner-ring substituents is removed (Jorgensen, E. C. Thyroid hormones and analogues. II. Structure-activity relationships. Hormonal Proteins and Peptides; Li, C. H., Ed.; Academic Press: New York: 1978; Vol. 6, pp 107-204). In turn, all thyromimetic compounds that have been moved into clinical development feature 3,5 inner ring di-substitution, including sobetirome (Elbers et al. Curr Atheroscler Rep (2016) 18: 14 DOI 10.1007/s11883-016-0564-7). However, as disclosed herein, properly-substituted thyromimetics can retain potency and show improved TRβ-selectivity vs T3 in the absence of one of these inner-ring substituents.

### BRIEF SUMMARY

Disclosed herein are compounds according to Formula I:

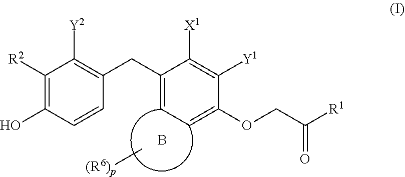

or a pharmaceutically acceptable isomer, racemate, tautomer, hydrate, solvate, isotope, or salt thereof, wherein B, X¹, Y¹, Y², R¹, R², R⁶, and p are as defined below.

Also disclosed herein are compounds according to Formula I′:

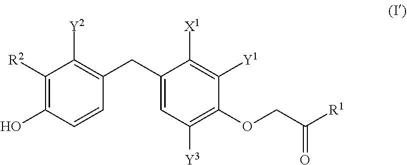

or a pharmaceutically acceptable isomer, racemate, tautomer, hydrate, solvate, isotope, or salt thereof, wherein R¹, R², X¹, Y¹, Y², and Y³ are as defined herein.

In an embodiment, a pharmaceutical composition is provided comprising a compound having the structure of Formula (I) or (I′), or a pharmaceutically acceptable isomer, racemate, tautomer, hydrate, solvate, isotope, or salt thereof, in combination with a pharmaceutically acceptable carrier, diluent, or excipient. In an embodiment, the pharmaceutical composition is for use in treating a neurodegenerative disorder including neurodegenerative disorders classified as a demyelinating disease such as X-linked adrenoleukodystrophy or multiple sclerosis. In another embodiment, the pharmaceutical composition is for use in treating a medical condition associated increased activity of TGF-β, such as a fibrotic disease.

In an embodiment, a method is provided for treating a neurodegenerative disorder in a subject in need thereof, comprising administering a compound having the structure of Formula (I) or (I′), or a pharmaceutically acceptable salt or composition comprising the same. In some aspects, the neurodegenerative disorder can be classified as a demyelinating disease such as X-linked adrenoleukodystrophy or multiple sclerosis.

In another embodiment, a method is provided for treating a medical condition associated with over-expression of TGF-β in a subject in need thereof, comprising administering a compound having the structure of Formula (I) or (I′), or a pharmaceutically acceptable salt or composition comprising the same. In some aspects, the medical condition associated with over-expression of TGF-β is a fibrotic disease.

### DETAILED DESCRIPTION

As mentioned above, the invention relates to thyromimetic compounds, to products comprising the same, and to methods for their use and synthesis.

In one embodiment, compounds are provided having the structure of Formula (I):

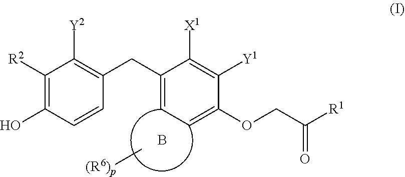

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl; R² is lower alkyl, lower alkenyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

The acid compounds of the present invention (R¹═—OR^{1c} and R^{1c}═H) are active agonists selectively activating the TRβ receptor. The amide compounds of the present invention (R¹═—NR^{1a}R^{1b}) may act as substrates for the specific hydrolase enzyme fatty acid-amide hydrolase (FAAH), which cleaves the amide, liberating the thyromimetic. Thus, prodrug conversion to drug is enhanced in tissues that express high levels of FAAH such as the central nervous system. The ester compounds of the present invention (R¹═—OR^{1c} and R^{1c}≠H) are also prodrugs, typically processed through the action of esterases which may exist selectively in specific tissues.

As used herein, “lower alkyl” means a straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of straight chain lower alkyl groups include, but are not limited to, methyl, ethyl, n-propyl, n-butyl, n-pentyl-, n-hexyl, n-heptyl, and n-octyl groups. Examples of branched lower alkyl groups include, but are not limited to, isopropyl, iso-butyl, sec-butyl, t-butyl, neopentyl, isopentyl, and 2,2-dimethylpropyl groups.

As used herein, “lower alkenyl” means a straight chain or branched alkenyl group having from 2 to 8 carbon atoms, in some embodiments from 2 to 6 carbon atoms, in some embodiments from 2 to 4 carbon atoms, and in some embodiments from 2 to 3 carbon atoms.

Alkenyl groups are unsaturated hydrocarbons that contain at least one carbon-carbon double bond. Examples of lower alkenyl groups include, but are not limited to, vinyl, propenyl, isopropenyl, butenyl, pentenyl, and hexenyl.

As used herein, “lower alkynyl” means a straight chain or branched alkynyl group having from 2 to 8 carbon atoms, in some embodiments from 2 to 6 carbon atoms, in some embodiments from 2 to 4 carbon atoms, and in some embodiments from 2 to 3 carbon atoms.

Alkynyl groups are unsaturated hydrocarbons that contain at least one carbon-carbon triple bond. Examples of lower alkynyl groups include, but are not limited to, ethynyl, propynyl, butynyl, pentynyl, and hexynyl.

“Halo” or “halogen” refers to fluorine, chlorine, bromine, and iodine.

“Hydroxy” refers to —OH.

“Cyano” refers to —CN.

“Lower haloalkyl” refers to a lower alkyl as defined above with one or more hydrogen atoms replaced with halo. Examples of lower haloalkyl groups include, but are not limited to, —CF₃, —CHF₂, and the like.

“Lower alkoxy” refers to a lower alkyl as defined above joined byway of an oxygen atom (i.e., —O-(lower alkyl). Examples of lower alkoxy groups include, but are not limited to, methoxy, ethoxy, n-propoxy, n-butoxy, isopropoxy, sec-butoxy, tert-butoxy, and the like.

“Lower haloalkoxy” refers to a lower haloalkyl as defined above joined by way of an oxygen atom (i.e., —O-(lower haloalkyl). Examples of lower haloalkoxy groups include, but are not limited to, —OCF₃, —OCHF₂, and the like.

“Cycloalkyl” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring do not give rise to aromaticity. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

“Cycloalkylalkyl” are alkyl groups as defined above in which a hydrogen or carbon bond of the alkyl group is replaced with a bond to a cycloalkyl group as defined above.

“Aryl” groups are cyclic aromatic hydrocarbons that do not contain heteroatoms. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl.

“Carbocyclyl,” “carbocycle,” or “carbocyclic” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring may give rise to aromaticity. In one embodiment, carbocycle includes cycloalkyl as defined above. In another embodiment, carbocycle includes aryl as defined above.

“Carbocyclealkyl” are alkyl groups as defined above in which a hydrogen or carbon bond of the alkyl group is replaced with a bond to a carbocycle group as defined above. Examples of carbocyclealkyl groups include, but are not limited to, cy clopropylmethyl, cyclobutylmethyl, benzyl, and the like.

“Heterocyclyl,” “heterocycle,” or “heterocyclic” refers to aromatic and non-aromatic ring moieties containing 3 or more ring members, of which one or more is a heteroatom such as, but not limited to, N, O, S, or P. In some embodiments, heterocyclyl include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, hydroxy, —CN, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

“Heterocyclealkyl” are alkyl groups as defined above in which a hydrogen or carbon bond of the alkyl group is replaced with a bond to a heterocycle group as defined above.

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

In one embodiment, compounds are provided having the structure of Formula (I) or (I′), or a pharmaceutically acceptable isomer, racemate, tautomer, hydrate, solvate, isotope, or salt thereof, wherein R² is lower alkyl optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl. In another embodiment, R² is unsubstituted lower alkyl. In a more specific embodiment, R² is methyl, ethyl, propyl, isopropyl, or butyl.

In one embodiment, compounds are provided having the structure of Formula (I), or a pharmaceutically acceptable isomer, racemate, tautomer, hydrate, solvate, isotope, or salt thereof, wherein B is carbocycle. In one embodiment, B is a 5-6 membered carbocycle. In another embodiment, B is a 5-6 membered cycloalkyl. In another embodiment, B is a 5-6 membered aryl.

In one embodiment, compounds are provided having the structure of Formula (I), or a pharmaceutically acceptable isomer, racemate, tautomer, hydrate, solvate, isotope, or salt thereof, wherein B is heterocycle. In one embodiment, B is a 5-6 membered heterocycle. In another embodiment, B is a 5-6 membered heterocycloalkyl. In another embodiment, B is a 5-6 membered heteroaryl.

In one embodiment, compounds are provided having the structure of Formula (I-A):

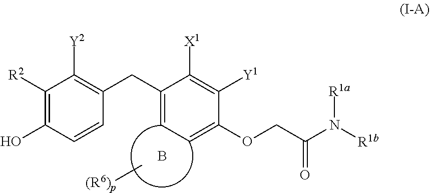

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R² is lower alkyl, lower alkenyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (I-A-1):

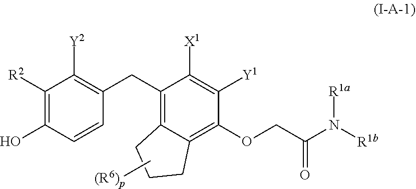

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R² is lower alkyl, lower alkenyl, carbocycle, heterocycle, carbocyclealkyl, or heterocyclealkyl;

each R⁶ is, independently, lower alkyl, lower alkenyl, lower haloalkyl, or halo; and

p is 0-3;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (I-A-2):

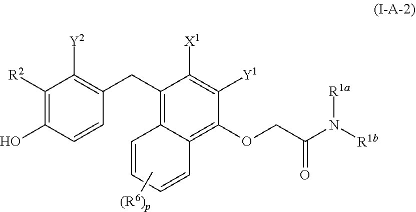

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R² is lower alkyl, lower alkenyl, carbocycle, heterocycle, carbocyclealkyl, or heterocyclealkyl;

each R⁶ is, independently, lower alkyl, lower alkenyl, lower haloalkyl, or halo; and

p is 0-3;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (I-B):

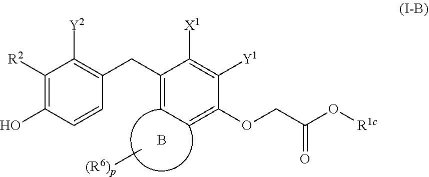

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R² is lower alkyl, lower alkenyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (I-B-1):

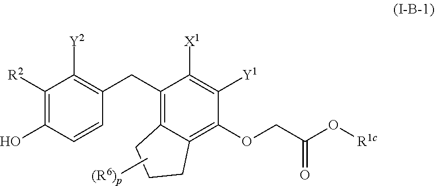

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R² is lower alkyl, lower alkenyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (I-B-2):

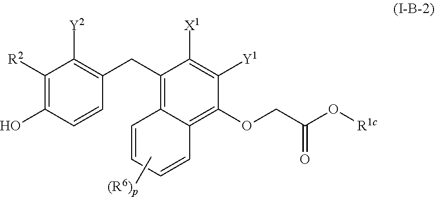

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R² is lower alkyl, lower alkenyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of any one of Formula (I), Formula (I-A), Formula (I-A-1), Formula (I-B), Formula (I-B-1), or Formula (I-B-2), or a pharmaceutically acceptable isomer, racemate, tautomer, hydrate, solvate, isotope, or salt thereof, wherein R² is lower alkyl optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl. In one embodiment, R² is unsubstituted lower alkyl. In one embodiment, R² is methyl, ethyl, propyl, or butyl.

In one embodiment, compounds are provided having the structure of Formula (II):

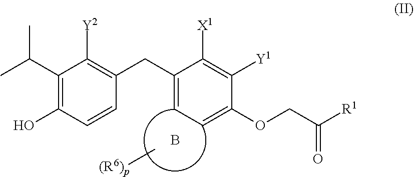

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy; R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle; and

R^{1c} is H, lower alkyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1a}, R^{1b}, and R^{1c} are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-A):

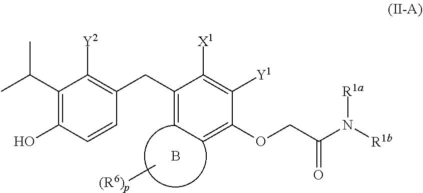

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁶ is, independently, lower alkyl, lower alkenyl, lower haloalkyl, or halo; and

p is 0-5;

wherein R^{1a} and R^{1b} are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-A-1):

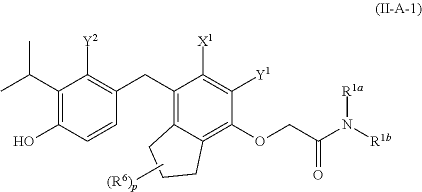

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁶ is, independently, lower alkyl, lower alkenyl, lower haloalkyl, or halo; and

p is 0-3;

wherein R^{1a} and R^{1b} are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-A-2):

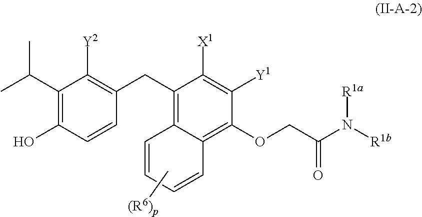

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁶ is, independently, lower alkyl, lower alkenyl, lower haloalkyl, or halo; and

p is 0-3;

wherein R^{1a} and R^{1b} are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-B):

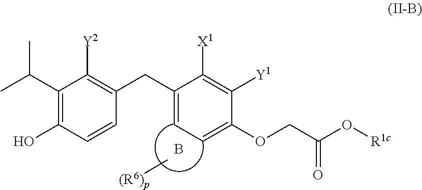

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1c} is optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-B-1):

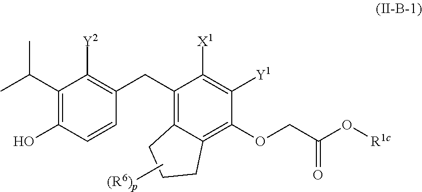

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1c} is optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-B-2):

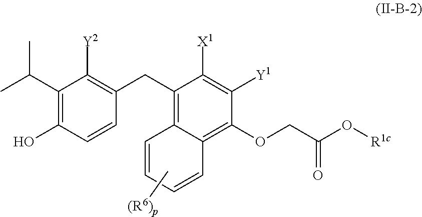

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁶ is, independently, lower alkyl, lower alkenyl, lower haloalkyl, or halo; and

p is 0-5;

wherein R^{1c} is optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III):

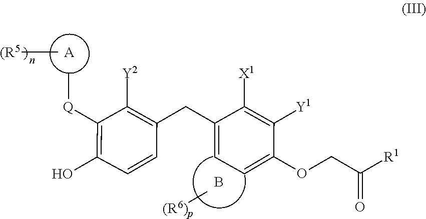

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy; Q is —C(R³R⁴)— or —{C(R³R⁴)}₂—;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle; and

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-A):

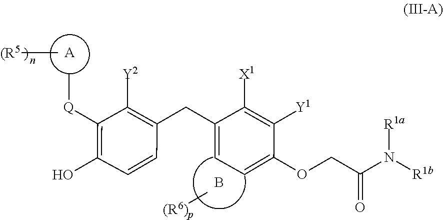

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

Q is —C(R³R⁴)— or —{C(R³R⁴)}₂—;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-A-1):

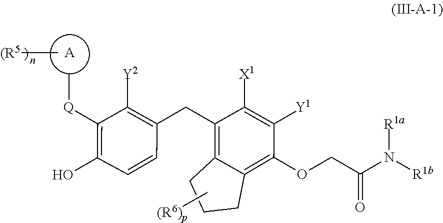

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

Q is —C(R³R⁴)— or —{C(R³R⁴)}₂—;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-A-2):

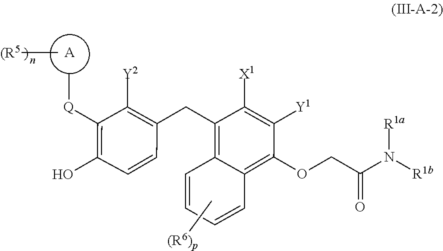

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

Q is —C(R³R⁴)— or —{C(R³R⁴)}₂—;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-B):

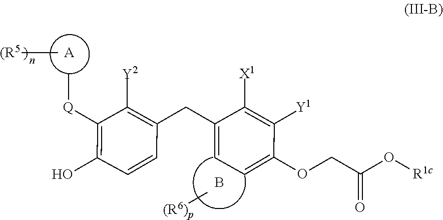

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

Q is —C(R³R⁴)— or —{C(R³R⁴)}₂—;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-B-1):

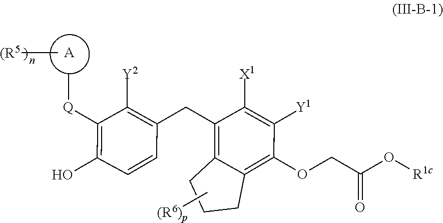

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

Q is —C(R³R⁴)— or —{C(R³R⁴)}₂—;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-B-2):

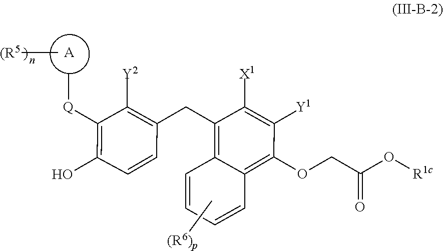

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

Q is —C(R³R⁴)— or —{C(R³R⁴)}₂—;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV):

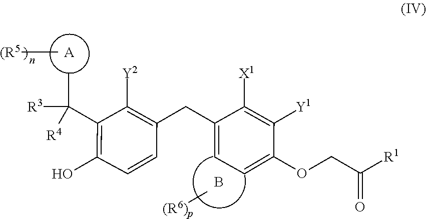

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;
n is 0-5; and
p is 0-5;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-A):

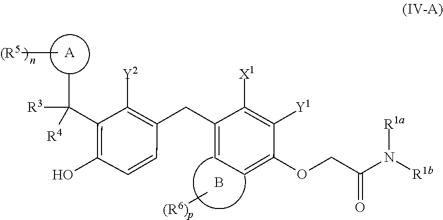

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-A-1):

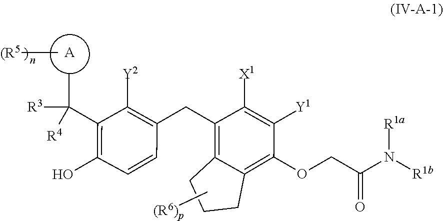

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-A-2):

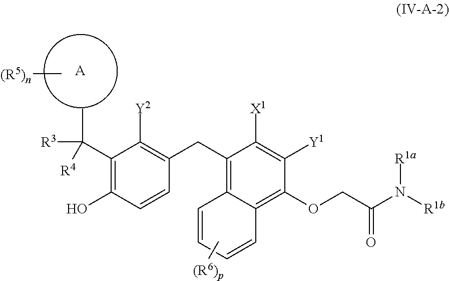

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-B):

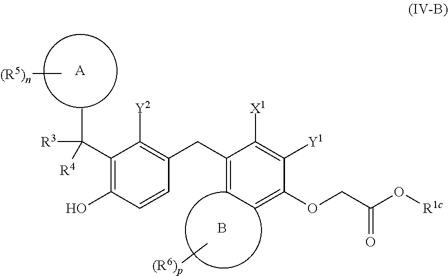

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-B-1):

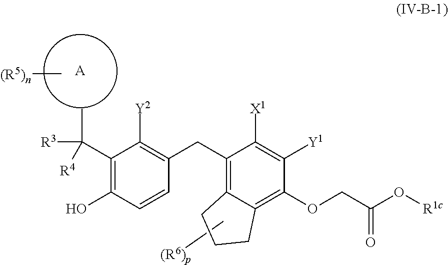

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy; Rao is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-B-2):

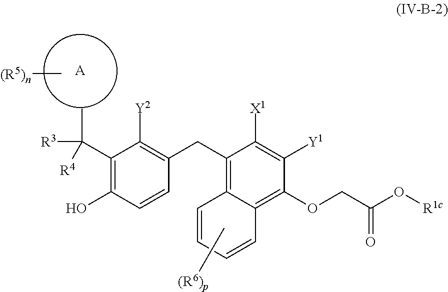

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V):

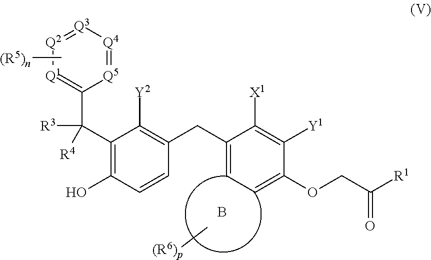

or a pharmaceutically acceptable isomer, racemate, tautomer, hydrate, solvate, isotope, or salt thereof, wherein:

Q¹, Q², Q³, Q⁴, and Q⁵ are each, independently, CH, CR⁵, or N;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-A):

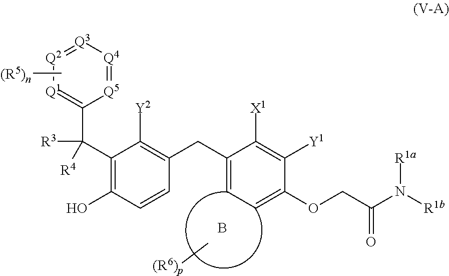

or a pharmaceutically acceptable isomer, racemate, tautomer, hydrate, solvate, isotope, or salt thereof, wherein:

Q¹, Q², Q³, Q⁴, and Q⁵ are each, independently, CH, CR⁵, or N;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-A-1):

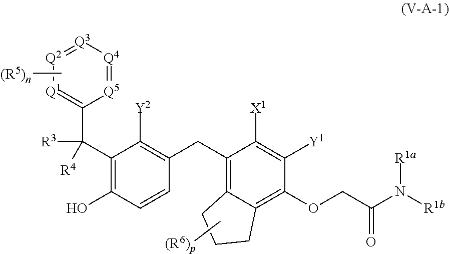

or a pharmaceutically acceptable isomer, racemate, tautomer, hydrate, solvate, isotope, or salt thereof, wherein:

Q¹, Q², Q³, Q⁴, and Q⁵ are each, independently, CH, CR⁵, or N;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-A-2):

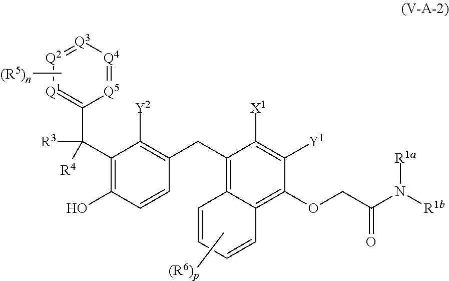

or a pharmaceutically acceptable isomer, racemate, tautomer, hydrate, solvate, isotope, or salt thereof, wherein:

Q¹, Q², Q³, Q⁴, and Q⁵ are each, independently, CH, CR⁵, or N;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-B):

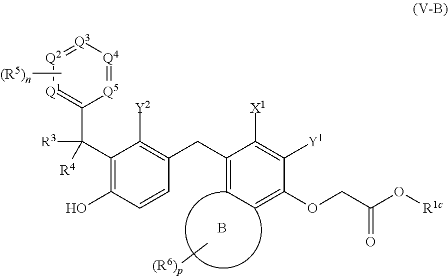

or a pharmaceutically acceptable isomer, racemate, tautomer, hydrate, solvate, isotope, or salt thereof, wherein:

Q¹, Q Q³, Q⁴, and Q⁵ are each, independently, CH, CR⁵, or N;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-B-1):

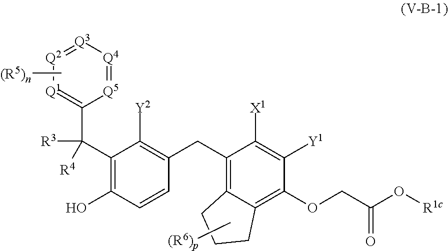

or a pharmaceutically acceptable isomer, racemate, tautomer, hydrate, solvate, isotope, or salt thereof, wherein:

Q¹, Q Q³, Q⁴, and Q⁵ are each, independently, CH, CR⁵, or N;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-B-2):

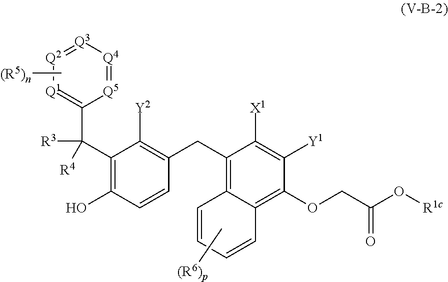

or a pharmaceutically acceptable isomer, racemate, tautomer, hydrate, solvate, isotope, or salt thereof, wherein:

Q¹, Q², Q³, Q⁴ and Q⁵ are each, independently, CH, CR, or N;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocycle alkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI):

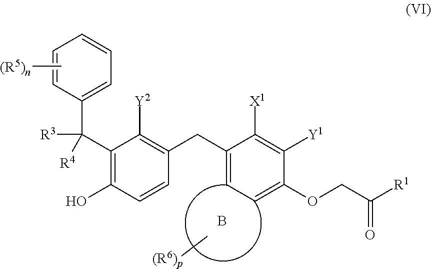

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI-A):

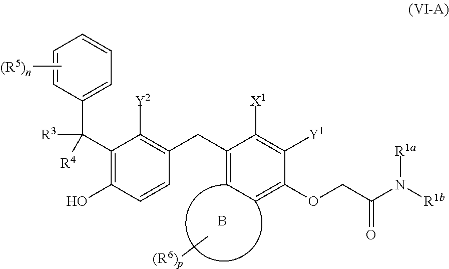

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI-A-1):

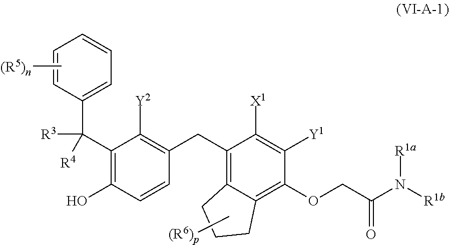

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI-A-2):

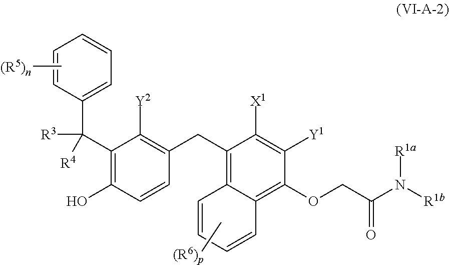

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI-B):

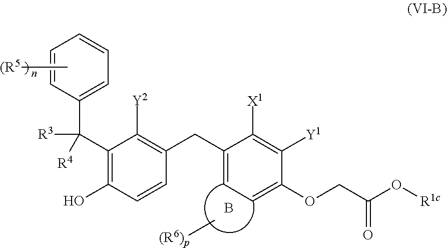

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI-B-1):

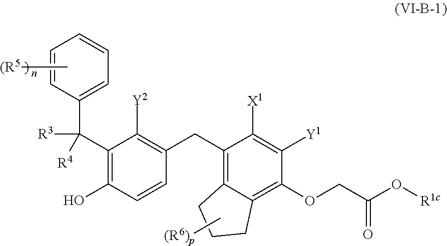

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI-B-2):

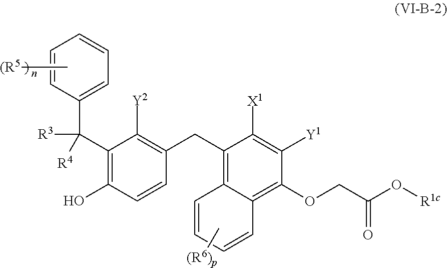

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII):

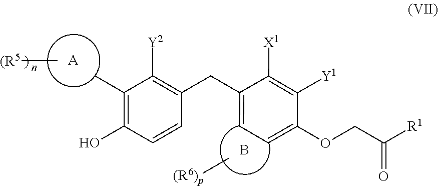

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-A):

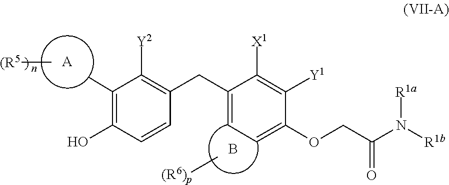

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-A-1):

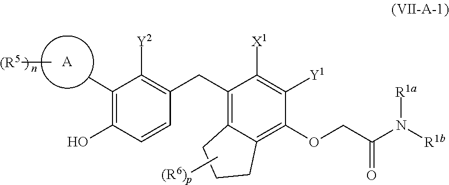

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-A-2):

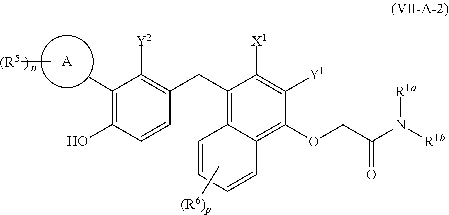

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-B):

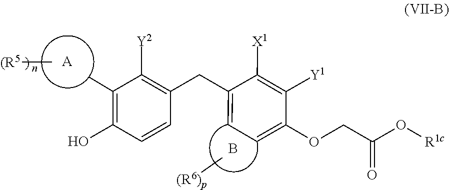

or a pharmaceutically acceptable isomer, racemate, tautomer, hydrate, solvate, isotope, or salt thereof, wherein:

A is aryl or heteroaryl;

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-B-1):

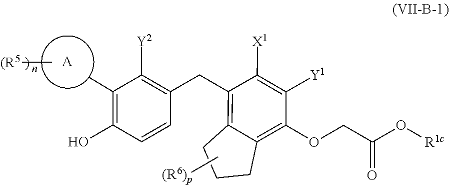

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-B-2):

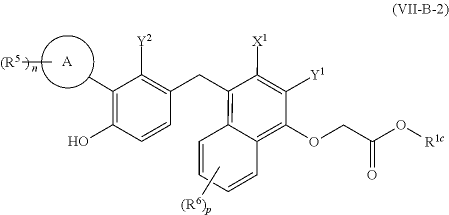

or a pharmaceutically acceptable isomer, racemate, tautomer, hydrate, solvate, isotope, or salt thereof, wherein:

A is aryl or heteroaryl;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII):

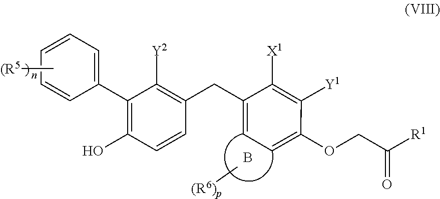

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-A):

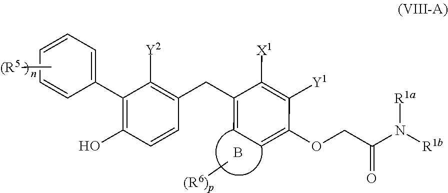

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-A-1):

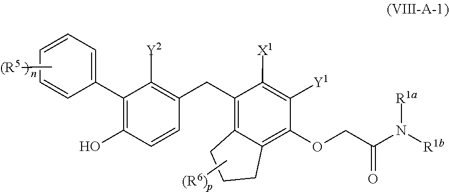

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-A-2):

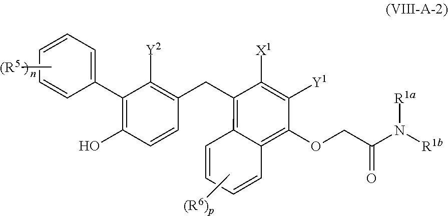

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-B):

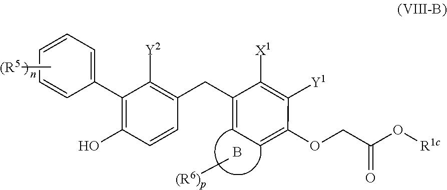

or a pharmaceutically acceptable isomer, racemate, tautomer, hydrate, solvate, isotope, or salt thereof, wherein:

B is a carbocyle or a heterocyle;

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-5;

wherein R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-B-1):

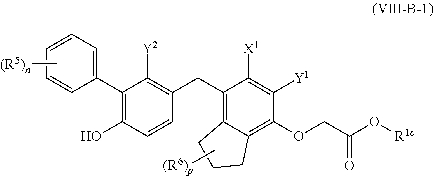

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-B-2):

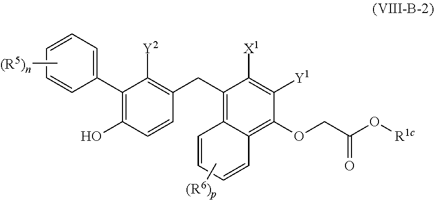

or a pharmaceutically acceptable isomer, racemate, tautomer, hydrate, solvate, isotope, or salt thereof, wherein:

X¹ is H, lower alkyl, lower alkenyl, lower haloalkyl, or halo;

Y¹ is H, —CN, halo, lower alkyl, or lower alkoxy;

Y² is H, —CN, halo, lower alkyl, or lower alkoxy;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

R^{a} and R^{b} are each, independently, H, lower alkyl, or lower haloalkyl;

each R⁶ is, independently, lower alkyl, lower alkenyl, lower haloalkyl, or halo;

n is 0-5; and

p is 0-3;

wherein R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), or Formula (VI-B-2), or a pharmaceutically acceptable isomer, racemate, tautomer, hydrate, solvate, isotope, or salt thereof, wherein R³ is H.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), or Formula (VI-B-2), or a pharmaceutically acceptable isomer, racemate, tautomer, hydrate, solvate, isotope, or salt thereof, wherein R³ is carbocycle. In one embodiment, R³ is cyclopropyl or cyclobutyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), or Formula (VI-B-2), or a pharmaceutically acceptable isomer, racemate, tautomer, hydrate, solvate, isotope, or salt thereof, wherein R³ is lower alkyl. In one embodiment, R³ is methyl, ethyl, or propyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), or Formula (VI-B-2), or a pharmaceutically acceptable isomer, racemate, tautomer, hydrate, solvate, isotope, or salt thereof, wherein R³ is —OR^{a}. In one embodiment, R^{a} is H. In one embodiment, R^{a} is lower alkyl. In one embodiment, R^{a} is lower methyl.

In one embodiment, compounds are provided having the structure of any one of Formula (I), Formula (II), Formula (III), Formula (IV), Formula (V), Formula (VI), Formula (VII), or Formula (VIII), or a pharmaceutically acceptable isomer, racemate, tautomer, hydrate, solvate, isotope, or salt thereof, wherein R¹ is R¹ is —NR^{1a}R^{1b}. In one embodiment, R¹ is —OR^{1c}.

In one embodiment, compounds are provided having the structure of any one of Formula (I), Formula (I-A), Formula (I-A-1), Formula (I-A-2), Formula (II), Formula (II-A), Formula (II-A-1), Formula (II-A-2), Formula (III), Formula (III-A), Formula (III-A-1), Formula (III-A-2), Formula (IV), Formula (IV-A), Formula (IV-A-1), Formula (IV-A-2), Formula (V), Formula (V-A), Formula (V-A-1), Formula (V-A-2), Formula (VI), Formula (VI-A), Formula (VI-A-1), Formula (VI-A-2), Formula (VII), Formula (VII-A), Formula (VII-A-1), Formula (VII-A-2), Formula (VIII), Formula (VIII-A), Formula (VIII-A-1), or Formula (VIII-A-2), or a pharmaceutically acceptable isomer, racemate, tautomer, hydrate, solvate, isotope, or salt thereof, wherein R^{1a} is lower alkyl. In one embodiment, R^{1a} is methyl.

In one embodiment, compounds are provided having the structure of any one of Formula (I), Formula (I-A), Formula (I-A-1), Formula (I-A-2), Formula (II), Formula (II-A), Formula (II-A-1), Formula (II-A-2), Formula (III), Formula (III-A), Formula (III-A-1), Formula (III-A-2), Formula (IV), Formula (IV-A), Formula (IV-A-1), Formula (IV-A-2), Formula (V), Formula (V-A), Formula (V-A-1), Formula (V-A-2), Formula (VI), Formula (VI-A), Formula (VI-A-1), Formula (VI-A-2), Formula (VII), Formula (VII-A), Formula (VII-A-1), Formula (VII-A-2), Formula (VIII), Formula (VIII-A), Formula (VIII-A-1), or Formula (VIII-A-2), or a pharmaceutically acceptable isomer, racemate, tautomer, hydrate, solvate, isotope, or salt thereof, wherein R^{1b} is H.

In one embodiment, compounds are provided having the structure of any one of Formula (I), Formula (I-B), Formula (I-B-1), Formula (I-B-2), Formula (II), Formula (II-B), Formula (II-B-1), Formula (II-B-2), Formula (III), Formula (III-B), Formula (III-B-1), Formula (III-B-2), Formula (IV), Formula (IV-B), Formula (IV-B-1), Formula (IV-B-2), Formula (V), Formula (V-B), Formula (V-B-1), Formula (V-B-2), Formula (VI), Formula (VI-B), Formula (VI-B-1), Formula (VI-B-2), Formula (VII), Formula (VII-B), Formula (VII-B-1), Formula (VII-B-2), Formula (VIII), Formula (VIII-B), Formula (VIII-B-1), or Formula (VIII-B-2), or a pharmaceutically acceptable isomer, racemate, tautomer, hydrate, solvate, isotope, or salt thereof, wherein R^{1c} is H.

In one embodiment, compounds are provided having the structure of any one of Formula (I), Formula (I-B), Formula (I-B-1), Formula (I-B-2), Formula (II), Formula (II-B), Formula (II-B-1), Formula (II-B-2), Formula (III), Formula (III-B), Formula (III-B-1), Formula (III-B-2), Formula (IV), Formula (IV-B), Formula (IV-B-1), Formula (IV-B-2), Formula (V), Formula (V-B), Formula (V-B-1), Formula (V-B-2), Formula (VI), Formula (VI-B), Formula (VI-B-1), Formula (VI-B-2), Formula (VII), Formula (VII-B), Formula (VII-B-1), Formula (VII-B-2), Formula (VIII), Formula (VIII-B), Formula (VIII-B-1), or Formula (VIII-B-2), or a pharmaceutically acceptable isomer, racemate, tautomer, hydrate, solvate, isotope, or salt thereof, wherein R^{1c} is lower alkyl. In one embodiment, R^{1c} is methyl or ethyl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein X¹ is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein X¹ lower alkyl. In one embodiment, X¹ is methyl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein X¹ is halo. In one embodiment, X¹ is Cl or Br. In one embodiment, X¹ is Cl. In another embodiment, X¹ is Br.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein X¹ is lower haloalkyl. In one embodiment, X¹ is —CH₂F, —CHF₂, or —CF₃. In one embodiment, X¹ is —CF₃.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein X¹ is lower alkenyl. In one embodiment, X¹ is vinyl or isopropenyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is lower alkyl. In one embodiment, R⁵ is lower alkyl substituted with —OR′. In one embodiment, R′ is H. In one embodiment, R′ is lower alkyl. In one embodiment, R′ is methyl, ethyl, or propyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is lower haloalkyl. In one embodiment, at least one R⁵ is —CH₂F, —CHF₂, or —CF₃. In one embodiment, at least one R⁵ is —CF₃.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is —OR^{a}. In one embodiment, R^{a} is lower alkyl. In one embodiment, R^{a} is methyl, ethyl, or propyl. In one embodiment, R^{a} is lower haloalkyl. In one embodiment, R^{a} is —CH₂F, —CHF₂, or —CF₃.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is —C(O)R^{a}. In one embodiment, R^{a} is lower alkyl. In one embodiment, R^{a} is methyl, ethyl, or propyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is —NR^{a}C(O)R^{b}. In one embodiment, R^{a} is H and R^{b} is lower alkyl. In one embodiment, R^{a} is H and R^{b} is methyl, ethyl, or propyl. In one embodiment, R^{a} is H and R^{b} is methyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is —C(O)OR^{a}. In one embodiment, R^{a} is lower alkyl. In one embodiment, R^{a} is methyl, ethyl, or propyl. In one embodiment, R^{a} is methyl. In one embodiment, R^{a} is ethyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is —S(O)₂R^{a}. In one embodiment, R^{a} is lower alkyl. In one embodiment, R^{a} is methyl, ethyl, or propyl. In one embodiment, R^{a} is methyl.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is halo. In one embodiment, at least one R⁵ is F.

In one embodiment, compounds are provided having the structure of any one of Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁵ is —CN.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is halo. In one embodiment, Y¹ is F or Cl. In one embodiment, Y¹ is F. In another embodiment, Y¹ is Cl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is —CN.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is lower alkyl. In one embodiment, Y¹ is methyl, ethyl, or propyl. In one embodiment, Y¹ is methyl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is lower alkoxy. In one embodiment, Y¹ is methoxy or ethoxy. In one embodiment, Y¹ is methoxy.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y² is halo. In one embodiment, Y² is F or Cl. In one embodiment, Y² is F. In one embodiment, Y² is Cl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y² is —CN.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y² is lower alkyl. In one embodiment, Y² is methyl, ethyl, or propyl. In one embodiment, Y² is methyl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y² is lower alkoxy. In one embodiment, Y² is methoxy or ethoxy. In one embodiment, Y² is methoxy.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y² is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is F and Y² is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is Cl and Y² is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is —CN and Y² is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is lower alkyl and Y² is H. In one embodiment, Y¹ is methyl and Y² is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is lower alkoxy and Y² is H. In one embodiment, Y¹ is methoxy and Y² is H.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is H and Y² is F.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is H and Y² is C1.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is H and Y² is —CN.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is H and Y² is lower alkyl. In one embodiment, Y¹ is H and Y² is methyl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is H and Y² is lower alkoxy. In one embodiment, Y¹ is H and Y² is methoxy.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein Y¹ is F and Y² is F.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein p is 0-3. In one embodiment, p is 0. In another embodiment, p is 1. In another embodiment, p is 2. In another embodiment, p is 3.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁶ is lower alkyl. In one embodiment, at least one R⁶ is methyl, ethyl, or propyl. In one embodiment, at least one R⁶ is methyl. In another embodiment, at least one R⁶ is ethyl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁶ is lower alkenyl. In one embodiment, at least one R⁶ is vinyl or isopropenyl.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁶ is lower haloalkyl. In one embodiment, at least one R⁶ is —CF₃, —CHF₂, or —CH₂F. In one embodiment, at least one R⁶ is —CF₃.

In one embodiment, compounds are provided having the structure of any one of Formula (I), Formula (I-A), Formula (I-A-1), Formula (I-A-2), Formula (I-B), Formula (I-B-1), Formula (I-B-2), Formula (II), Formula (II-A), Formula (II-A-1), Formula (II-A-2), Formula (II-B), Formula (II-B-1), Formula (II-B-2), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V), Formula (V-A), Formula (V-A-1), Formula (V-A-2), Formula (V-B), Formula (V-B-1), Formula (V-B-2), Formula (VI), Formula (VI-A), Formula (VI-A-1), Formula (VI-A-2), Formula (VI-B), Formula (VI-B-1), Formula (VI-B-2), Formula (VII), Formula (VII-A), Formula (VII-A-1), Formula (VII-A-2), Formula (VII-B), Formula (VII-B-1), Formula (VII-B-2), Formula (VIII), Formula (VIII-A), Formula (VIII-A-1), Formula (VIII-A-2), Formula (VIII-B), Formula (VIII-B-1), or Formula (VIII-B-2), or a pharmaceutically acceptable isomer, racemate, tautomer, hydrate, solvate, isotope, or salt thereof, wherein at least one R⁶ is halo. In one embodiment, at least one R⁶ is F. In one embodiment, at least one R⁶ is Cl.

Representative compounds of Formula (I), and Formulas (II) through (VIII) as applicable, include the compounds listed in Table 1 below, as well as pharmaceutically acceptable salts thereof. To this end, representative compounds are identified herein by their respective “Compound Number”, which is sometimes abbreviated as “Compound No.”, “Cmpd. No.” or “No.”

TABLE 1
Representative Compounds

| Cmpd | | |
|---|---|---|
| No | Structure | Name |
| 1 | 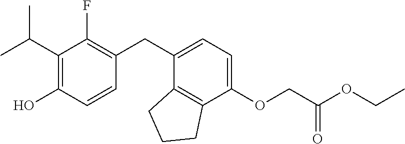 [SMILES: CCOC(=O)COc1ccc(Cc2ccc(O)c(C(C)C)c2F)c2c1CCC2] | ethyl 2-((7-(2-fluoro-4- hydroxy-3- isopropylbenzyl)-2,3- dihydro-1H-inden-4- yl)oxy)acetate |
| 2 | 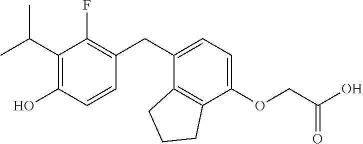 [SMILES: CC(C)c1c(O)ccc(Cc2ccc(OCC(=O)O)c3c2CCC3)c1F] | 2-((7-(2-fluoro-4-hydroxy- 3-isopropylbenzyl)-2,3- dihydro-1H-inden-4- yl)oxy)acetic acid |
| 3 | 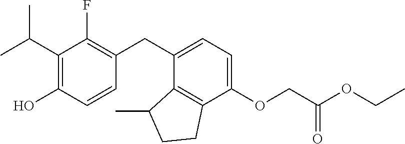 [SMILES: CCOC(=O)COc1ccc(Cc2ccc(O)c(C(C)C)c2F)c2c1CCC2C] | ethyl 2-((7-(2-fluoro-4- hydroxy-3- isopropylbenzyl)-1-methyl- 2,3-dihydro-1H-inden-4- yl)oxy)acetate |
| 4 | 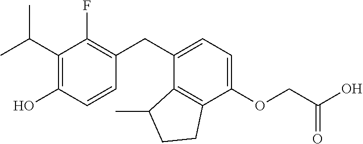 [SMILES: CC(C)c1c(O)ccc(Cc2ccc(OCC(=O)O)c3c2C(C)CC3)c1F] | 2-((7-(2-fluoro-4-hydroxy- 3-isopropylbenzyl)-1- methyl-2,3-dihydro-1H- inden-4-yl)oxy)acetic acid |
| 5 | 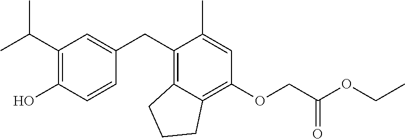 [SMILES: CCOC(=O)COc1cc(C)c(Cc2ccc(O)c(C(C)C)c2)c2c1CCC2] | ethyl 2-((7-(4-hydroxy-3- isopropylbenzyl)-6-methyl- 2,3-dihydro-1H-inden-4- yl)oxy)acetate |
| 6 | 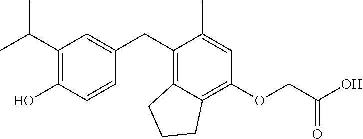 [SMILES: Cc1cc(OCC(=O)O)c2c(c1Cc1ccc(O)c(C(C)C)c1)CCC2] | 2-((7-(4-hydroxy-3- isopropylbenzyl)-6-methyl- 2,3-dihydro-1H-inden-4- yl)oxy)acetic acid |
| 7 | 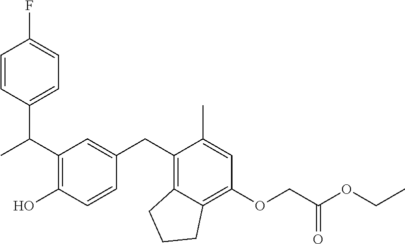 [SMILES: CCOC(=O)COc1cc(C)c(Cc2ccc(O)c(C(C)c3ccc(F)cc3)c2)c2c1CCC2] | ethyl 2-((7-(3-(1-(4- fluorophenyl)ethyl)-4- hydroxybenzyl)-6-methyl- 2,3-dihydro-1H-inden-4- yl)oxy)acetate |
| 8 | 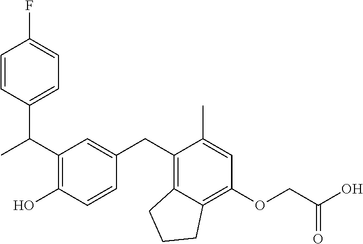 [SMILES: Cc1cc(OCC(=O)O)c2c(c1Cc1ccc(O)c(C(C)c3ccc(F)cc3)c1)CCC2] | 2-((7-(3-(1-(4- fluorophenyl)ethyl)-4- hydroxybenzyl)-6-methyl- 2,3-dihydro-1H-inden-4- yl)oxy)acetic acid |
| 9 | 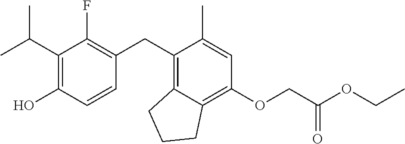 [SMILES: CCOC(=O)COc1cc(C)c(Cc2ccc(O)c(C(C)C)c2F)c2c1CCC2] | ethyl 2-((7-(2-fluoro-4- hydroxy-3- isopropylbenzyl)-6-methyl- 2,3-dihydro-1H-inden-4- yl)oxy)acetate |
| 10 | 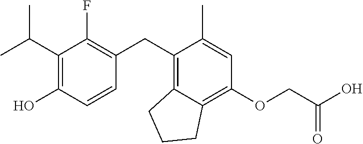 [SMILES: Cc1cc(OCC(=O)O)c2c(c1Cc1ccc(O)c(C(C)C)c1F)CCC2] | 2-((7-(2-fluoro-4-hydroxy- 3-isopropylbenzyl)-6- methyl-2,3-dihydro-1H- inden-4-yl)oxy)acetic acid |
| 11 | 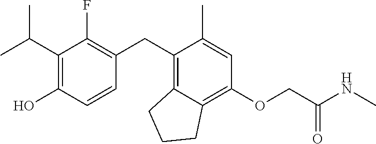 [SMILES: CNC(=O)COc1cc(C)c(Cc2ccc(O)c(C(C)C)c2F)c2c1CCC2] | 2-((7-(2-fluoro-4-hydroxy- 3-isopropylbenzyl)-6- methyl-2,3-dihydro-1H- inden-4-yl)oxy)-N- methylacetamide |
| 12 | 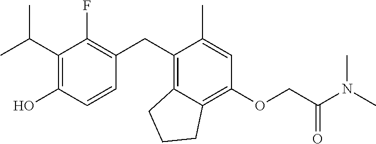 [SMILES: Cc1cc(OCC(=O)N(C)C)c2c(c1Cc1ccc(O)c(C(C)C)c1F)CCC2] | 2-((7-(2-fluoro-4-hydroxy- 3-isopropylbenzyl)-6- methyl-2,3-dihydro-1H- inden-4-yl)oxy)-N,N- dimethylacetamide |
| 13 | 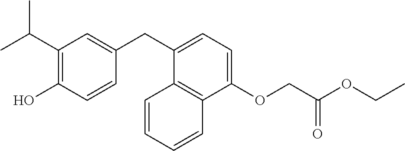 [SMILES: CCOC(=O)COc1ccc(Cc2ccc(O)c(C(C)C)c2)c2ccccc12] | ethyl 2-((4-(4-hydroxy-3- isopropylbenzyl)naphthalen- 1-yl)oxy)acetate |
| 14 | 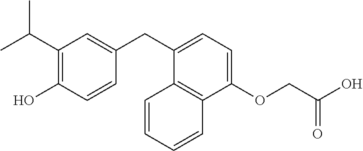 [SMILES: CC(C)c1cc(Cc2ccc(OCC(=O)O)c3ccccc23)ccc1O] | 2-((4-(4-hydroxy-3- isopropylbenzyl)naphthalen- 1-yl)oxy)acetic acid |
| 15 | 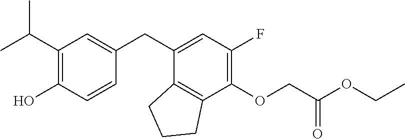 [SMILES: CCOC(=O)COc1c(F)cc(Cc2ccc(O)c(C(C)C)c2)c2c1CCC2] | ethyl 2-((5-fluoro-7-(4- hydroxy-3- isopropylbenzyl)-2,3- dihydro-1H-inden-4- yl)oxy)acetate |
| 16 | 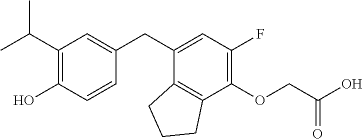 [SMILES: CC(C)c1cc(Cc2cc(F)c(OCC(=O)O)c3c2CCC3)ccc1O] | 2-((5-fluoro-7-(4-hydroxy- 3-isopropylbenzyl)-2,3- dihydro-1H-inden-4- yl)oxy)acetic acid |

In one embodiment, compounds are provided having the structure of Formula (I′):

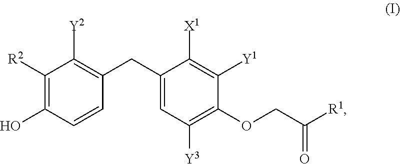

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl; and

R² is lower alkyl, lower alkenyl, carbocycle, heterocycle, carbocyclealkyl, or heterocyclealkyl;

wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

The acid compounds of the present invention (R¹═—OR^{1c} and R^{1c}═H) are active agonists selectively activating the TRβ receptor. The amide compounds of the present invention (R¹═—NR^{1a}R^{1b}) may act as substrates for the specific hydrolase enzyme fatty acid-amide hydrolase (FAAH), which cleaves the amide, liberating the thyromimetic. Thus, prodrug conversion to drug is enhanced in tissues that express high levels of FAAH such as the central nervous system. The ester compounds of the present invention (R¹═—OR^{1c} and R^{1c} H) are also prodrugs, typically processed through the action of esterases which may exist selectively in specific tissues.

In one embodiment, compounds are provided having the structure of Formula (II′):

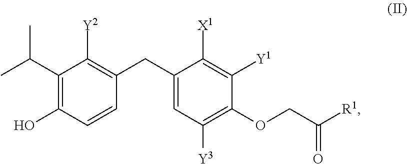

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl; and wherein R^{1a}, R^{1b}, and R^{1c} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-A′):

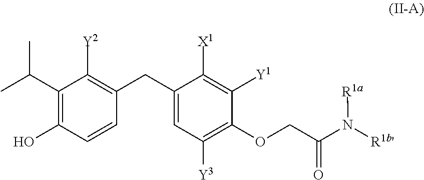

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy; and

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle; wherein R^{1a} and R^{1b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (II-B′):

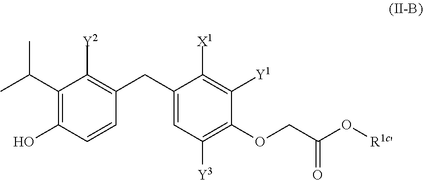

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy; and

R^{1c} is H, lower alkyl, carbocycle, heterocycle, carbocyclealkyl, or heterocyclealkyl; wherein R^{1c} is optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds of Formula (I) are provided wherein R² is carbocyclealkyl or heterocyclealkyl, or a pharmaceutically acceptable isomer, racemate, tautomer, hydrate, solvate, isotope, or salt thereof. In another embodiment, compounds are provided having the structure of Formula (III′):

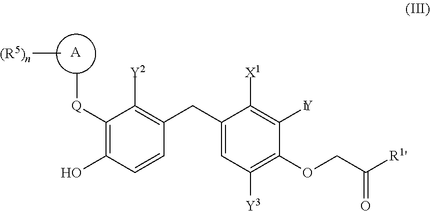

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

Q is —C(R³R⁴)— or —{C(R³R⁴)}₂—;

A is aryl or heteroaryl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-A′):

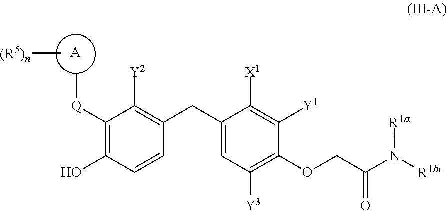

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

Q is —C(R³R⁴)— or —{C(R³R⁴)}₂—;

A is aryl or heteroaryl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (III-B′):

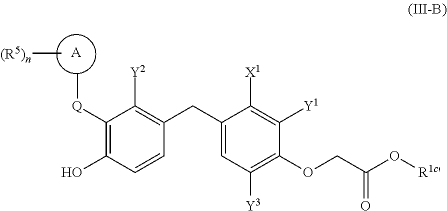

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1c} is H, lower alkyl, carbocycle, heterocycle, carbocyclealkyl, or heterocyclealkyl;

Q is —C(R³R⁴)— or —{C(R³R⁴)}₂—;

A is aryl or heteroaryl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV′):

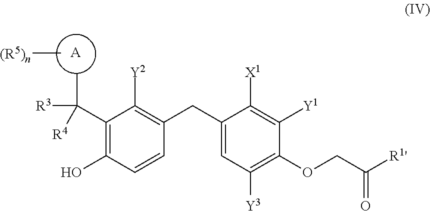

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

A is aryl or heteroaryl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-A′):

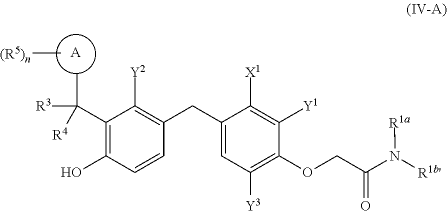

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

A is aryl or heteroaryl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (IV-B′):

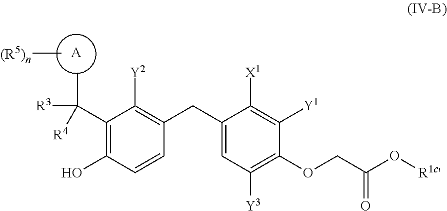

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1c} is H, lower alkyl, carbocycle, heterocycle, carbocyclealkyl, or heterocyclealkyl;

A is aryl or heteroaryl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V′):

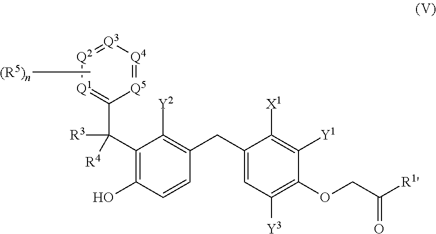

or a pharmaceutically acceptable isomer, racemate, tautomer, hydrate, solvate, isotope, or salt thereof, wherein:

Q¹, Q², Q³, Q⁴, and Q⁵ are each, independently, CH, CR, or N;

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-A′):

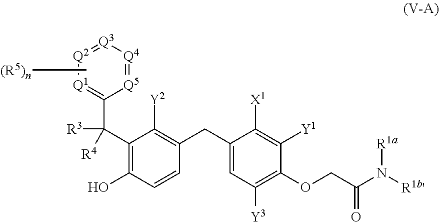

or a pharmaceutically acceptable isomer, racemate, tautomer, hydrate, solvate, isotope, or salt thereof, wherein:

Q¹, Q², Q³, Q⁴, and Q⁵ are each, independently, CH, CR⁵, or N;

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (V-B′):

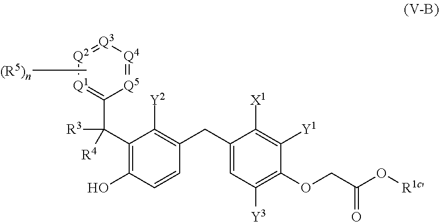

or a pharmaceutically acceptable isomer, racemate, tautomer, hydrate, solvate, isotope, or salt thereof, wherein:

Q¹, Q², Q³, Q⁴, and Q⁵ are each, independently, CH, CR⁵, or N;

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI′):

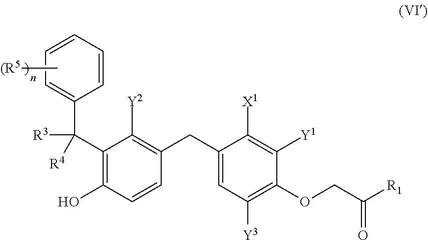

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VI-A′):

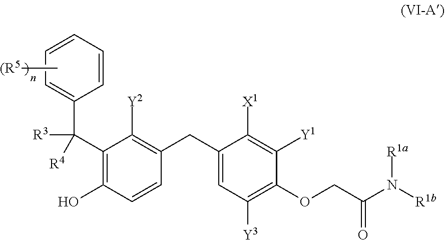

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}R^{1b}R³, R⁴, R⁵, R^{a}, and R^{b} are each, independently, optionally substituted with one or more halo, —CN, —OR, —NR′R″, ═O, ═S, —S(c)₂R′ or —S(O)₂OR′, wherein R′ and R″ are each, independently, H, lower alkyl, or lower haloalkyl.

In one embodiment, compounds are provided having the structure of Formula (VI-B′):

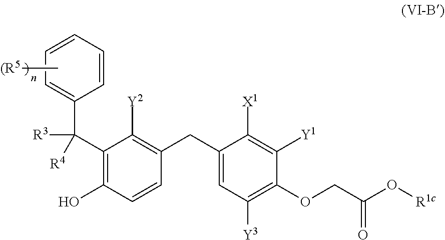

or a pharmaceutically acceptable isomer, racemate, tautomer, hydrate, solvate, isotope, or salt thereog, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1c} is H, lower alkyl, carbocycle, heterocycle, carbocyclealkyl, or heterocyclealkyl;

R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII′):

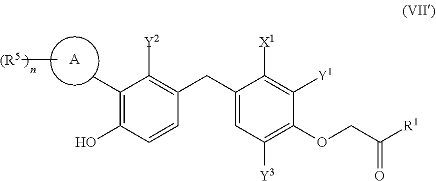

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

A is aryl or heteroaryl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-A′):

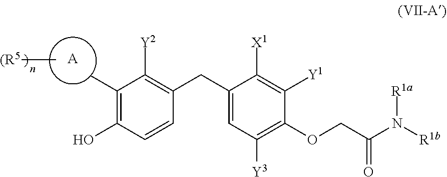

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

A is aryl or heteroaryl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VII-B′):

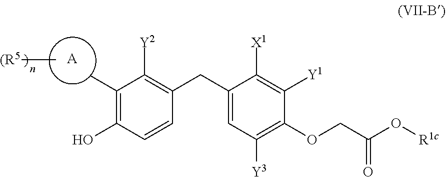

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1c} is H, lower alkyl, carbocycle, heterocycle, carbocyclealkyl, or heterocyclealkyl;

A is aryl or heteroaryl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII′):

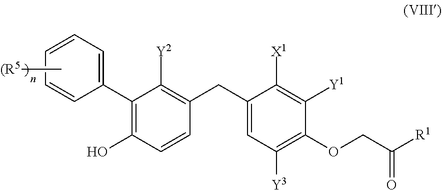

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R¹ is —NR^{1a}R^{1b} or —OR^{1c};

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-A′):

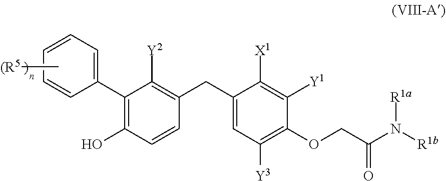

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of Formula (VIII-B′):

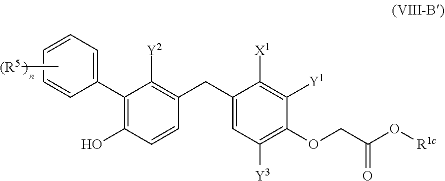

or a pharmaceutically acceptable isomer, racemate, tautomer, hydrate, solvate, isotope, or salt thereof, wherein:

X¹ is lower alkyl, lower alkenyl, lower haloalkyl, or halo;

Y¹ is H, —CN, halogen, lower alkyl, or lower alkoxy;

Y² is H or halogen;

Y³ is H, —CN, halogen, lower alkyl, or lower alkoxy;

R^{1c} is H, lower alkyl, carbocycle, heterocycle, carbocyclealkyl, or heterocyclealkyl;

each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};

n is 0-5; and

R^{a} and R^{b} are each, independently, H, lower alkyl, or lower haloalkyl;

wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), or a pharmaceutically acceptable isomer, racemate, tautomer, hydrate, solvate, isotope, or salt thereof, wherein R³ is H.

In one embodiment, compounds are provided having the structure of Formula (III′), Formula (III-A′), Formula (III-B′), Formula (IV′), Formula (IV-A′), Formula (IV-B′), Formula (V′), Formula (V-A′), or a pharmaceutically acceptable isomer, racemate, tautomer, hydrate, solvate, isotope, or salt thereof, wherein R³ is carbocycle. In one embodiment, R³ is cyclopropyl or cyclobutyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), or a pharmaceutically acceptable isomer, racemate, tautomer, hydrate, solvate, isotope, or salt thereof, wherein R³ is lower alkyl. In one embodiment, R³ is methyl, ethyl, or propyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), or a pharmaceutically acceptable isomer, racemate, tautomer, hydrate, solvate, isotope, or salt thereof, wherein R³ is —OR^{a}. In one embodiment, R^{a} is H. In one embodiment, R^{a} is lower alkyl. In a more specific embodiment, R^{a} is methyl.

In one embodiment, compounds are provided having the structure of any one of Formula (I′), Formula (II′), Formula (II-A′), Formula (III′), Formula (III-A′), Formula (IV′), Formula (IV-A′), Formula (V′), Formula (V-A′), Formula (VI′), Formula (VI-A′), Formula (VII′), Formula (VII-A′), Formula (VIII′), Formula (VIII-A′), or a pharmaceutically acceptable isomer, racemate, tautomer, hydrate, solvate, isotope, or salt thereof, wherein R¹ is —NR^{1a}R^{1b} and R^{1b} is H.

In one embodiment, compounds are provided having the structure of any one of Formula (I′), Formula (II′), Formula (II-A′), Formula (III′), Formula (III-A′), Formula (IV′), Formula (IV-A′), Formula (V′), Formula (V-A′), Formula (VI′), Formula (VI-A′), Formula (VII′), Formula (VII-A′), Formula (VIII′), Formula (VIII-A′), or a pharmaceutically acceptable isomer, racemate, tautomer, hydrate, solvate, isotope, or salt thereof, wherein R¹ is —NR^{1a}R^{1b} and R^{1a} is H. In one embodiment, R¹ is —NR^{1a}R^{1b} and R^{1a} is lower alkyl. In one embodiment, R¹ is —NR^{1a}R^{1b} and R^{1a} is methyl.

In one embodiment, compounds are provided having the structure of any one of Formula (I′), Formula (II′), Formula (II-B′), Formula (III′), Formula (III-B′), Formula (IV′), Formula (IV-B′), Formula (V′), Formula (V-B′), Formula (VI′), Formula (VI-B′), Formula (VII′), Formula (VII-B′), Formula (VIII′), Formula (VIII-B′), or a pharmaceutically acceptable isomer, racemate, tautomer, hydrate, solvate, isotope, or salt thereof, wherein R¹ is —OR^{1c} and R^{1c} is H.

In one embodiment, compounds are provided having the structure of any one of Formula (I′), Formula (II′), Formula (II-B′), Formula (III′), Formula (III-B′), Formula (IV′), Formula (IV-B′), Formula (V′), Formula (V-B′), Formula (VI′), Formula (VI-B′), Formula (VII′), Formula (VII-B′), Formula (VIII′), Formula (VIII-B′), or a pharmaceutically acceptable isomer, racemate, tautomer, hydrate, solvate, isotope, or salt thereof, wherein R¹ is —OR^{1c} and R^{1c} is lower alkyl. In one embodiment, R^{1c} is methyl or ethyl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein X¹ is lower alkyl. In one embodiment, X¹ is methyl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein X¹ is halo. In one embodiment, X¹ is Cl or Br. In one embodiment, X¹ is Cl. In one embodiment, X¹ is Br.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein X¹ is lower haloalkyl. In one embodiment, X¹ is —CF₃, —CHF₂, or —CH₂F.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein X¹ is lower alkenyl. In one embodiment, X¹ is vinyl. In another embodiment, X¹ is isopropenyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is lower alkyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is lower alkyl substituted with —OR′. In one embodiment, R′ is H. In another embodiment, R′ is lower alkyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is lower haloalkyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is —OR^{a}. In one embodiment, R^{a} is lower alkyl. In one embodiment, R^{a} is lower haloalkyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is —C(O)R^{a}. In one embodiment, R^{a} is lower alkyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is —NR^{a}C(O)R^{b}. In one embodiment, R^{a} is H and R^{b} is lower alkyl. In one embodiment, R^{b} is methyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is —C(O)OR^{a}. In one embodiment, R^{a} is lower alkyl. In one embodiment, R^{a} is methyl or ethyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is —S(O)₂R^{a}. In one embodiment, R^{a} is lower alkyl. In one embodiment, R^{a} is methyl.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is halo. In one embodiment, at least one R⁵ is F.

In one embodiment, compounds are provided having the structure of any one of Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein at least one R⁵ is —CN.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is halogen. In one embodiment, Y¹ is F. In one embodiment, Y¹ is Cl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is —CN.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is lower alkyl. In one embodiment, Y¹ is methyl, ethyl, or isopropyl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is lower alkoxy. In one embodiment, Y¹ is methoxy.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is H.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y² is halogen. In one embodiment, Y² is F. In one embodiment, Y² is Cl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y² is —CN.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y² is lower alkyl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y² is lower alkoxy.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y² is H.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y³ is halogen. In one embodiment, Y³ is F. In one embodiment, Y³ is Cl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y³ is —CN.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y³ is lower alkyl. In one embodiment, Y³ is methyl, ethyl, or isopropyl.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y³ is lower alkoxy. In one embodiment, Y³ is methoxy.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y³ is H.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is H, Y² is H, and Y³ is F.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is H, Y² is F, and Y³ is H.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is F, Y² is F, and Y³ is H.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI′), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is H, Y² is F, and Y³ is F.

In one embodiment, compounds are provided having the structure of any one of Formula (I′), Formula (II′), Formula (II-A′), Formula (II-B′), Formula (III′), Formula (III-A′), Formula (III-B′), Formula (IV′), Formula (IV-A′), Formula (IV-B′), Formula (V′), Formula (V-A′), Formula (V-B′), Formula (VI), Formula (VI-A′), Formula (VI-B′), Formula (VII′), Formula (VII-A′), Formula (VII-B′), Formula (VIII′), Formula (VIII-A′), Formula (VIII-B′), or a pharmaceutically acceptable isomer, racemate, tautomer, hydrate, solvate, isotope, or salt thereof, wherein Y¹ is F, Y² is H, and Y³ is F.

Representative compounds of Formula (I′), and Formulas (II′) through (VIII-B′) as applicable, include the compounds listed in Table 2 below, as well as pharmaceutically acceptable salts thereof. To this end, representative compounds are identified herein b y their respective “Compound Number”, which is sometimes abbreviated as “Compound No.”, “Cmpd. No.” or “No.”

TABLE 2
Representative Compounds

| Cmpd | | |
|---|---|---|
| No | Structure | Name |
| 17 | 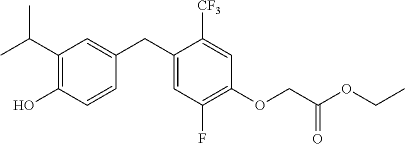 [SMILES: CCOC(=O)COc1cc(C(F)(F)F)c(Cc2ccc(O)c(C(C)C)c2)cc1F] | ethyl 2-(2-fluoro-4-(4-hydroxy-3- isopropylbenzyl)-5- (trifluoromethyl)phenoxy)acetate |
| 18 | 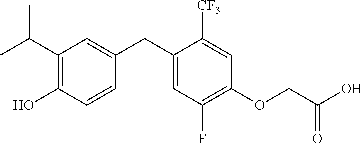 [SMILES: CC(C)c1cc(Cc2cc(F)c(OCC(=O)O)cc2C(F)(F)F)ccc1O] | 2-(2-fluoro-4-(4-hydroxy-3- isopropylbenzyl)-5- (trifluoromethyl)phenoxy)acetic acid |
| 19 | 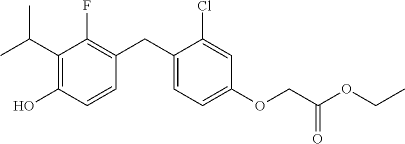 [SMILES: CCOC(=O)COc1ccc(Cc2ccc(O)c(C(C)C)c2F)c(Cl)c1] | ethyl 2-(3-chloro-4-(2-fluoro-4- hydroxy-3- isopropylbenzyl)phenoxy)acetate |
| 20 | 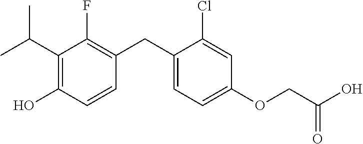 [SMILES: CC(C)c1c(O)ccc(Cc2ccc(OCC(=O)O)cc2Cl)c1F] | 2-(3-chloro-4-(2-fluoro-4-hydroxy- 3-isopropylbenzyl)phenoxy)acetic acid |
| 21 | 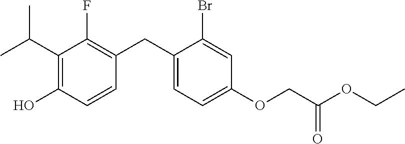 [SMILES: CCOC(=O)COc1ccc(Cc2ccc(O)c(C(C)C)c2F)c(Br)c1] | ethyl 2-(3-bromo-4-(2-fluoro-4- hydroxy-3- isopropylbenzyl)phenoxy)acetate |
| 22 | 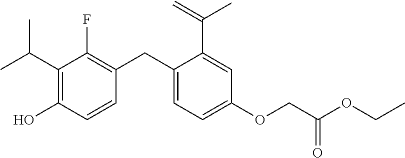 [SMILES: C=C(C)c1cc(OCC(=O)OCC)ccc1Cc1ccc(O)c(C(C)C)c1F] | ethyl 2-(4-(2-fluoro-4-hydroxy-3- isopropylbenzyl)-3-(prop-1-en-2- yl)phenoxy)acetate |
| 23 | 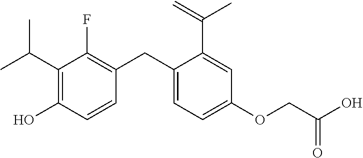 [SMILES: C=C(C)c1cc(OCC(=O)O)ccc1Cc1ccc(O)c(C(C)C)c1F] | 2-(4-(2-fluoro-4-hydroxy-3- isopropylbenzyl)-3-(prop-1-en-2- yl)phenoxy)acetic acid |
| 24 | 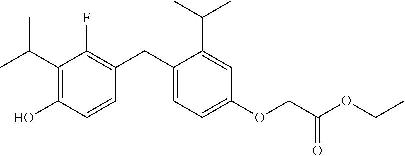 [SMILES: CCOC(=O)COc1ccc(Cc2ccc(O)c(C(C)C)c2F)c(C(C)C)c1] | ethyl 2-(4-(2-fluoro-4-hydroxy-3- isopropylbenzyl)-3- isopropylphenoxy)acetate |
| 25 | 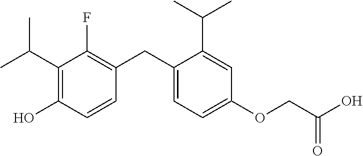 [SMILES: CC(C)c1cc(OCC(=O)O)ccc1Cc1ccc(O)c(C(C)C)c1F] | 2-(4-(2-fluoro-4-hydroxy-3- isopropylbenzyl)-3- isopropylphenoxy)acetic acid |
| 26 | 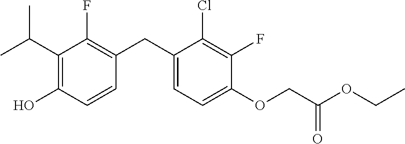 [SMILES: CCOC(=O)COc1ccc(Cc2ccc(O)c(C(C)C)c2F)c(Cl)c1F] | ethyl 2-(3-chloro-2-fluoro-4-(2- fluoro-4-hydroxy-3- isopropylbenzyl)phenoxy)acetate |
| 27 | 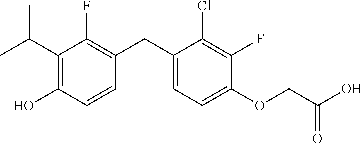 [SMILES: CC(C)c1c(O)ccc(Cc2ccc(OCC(=O)O)c(F)c2Cl)c1F] | 2-(3-chloro-2-fluoro-4-(2-fluoro-4- hydroxy-3- isopropylbenzyl)phenoxy)acetic acid |
| 28 | 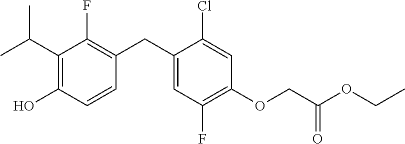 [SMILES: CCOC(=O)COc1cc(Cl)c(Cc2ccc(O)c(C(C)C)c2F)cc1F] | ethyl 2-(5-chloro-2-fluoro-4-(2- fluoro-4-hydroxy-3- isopropylbenzyl)phenoxy)acetate |
| 29 | 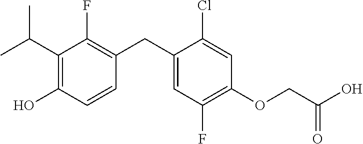 [SMILES: CC(C)c1c(O)ccc(Cc2cc(F)c(OCC(=O)O)cc2Cl)c1F] | 2-(5-chloro-2-fluoro-4-(2-fluoro-4- hydroxy-3- isopropylbenzyl)phenoxy)acetic acid |
| 30 | 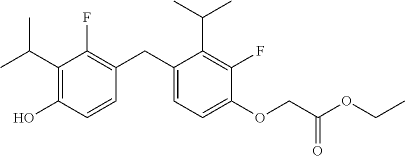 [SMILES: CCOC(=O)COc1ccc(Cc2ccc(O)c(C(C)C)c2F)c(C(C)C)c1F] | ethyl 2-(2-fluoro-4-(2-fluoro-4- hydroxy-3-isopropylbenzyl)-3- isopropylphenoxy)acetate |
| 31 | 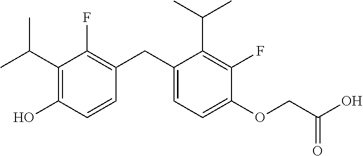 [SMILES: CC(C)c1c(O)ccc(Cc2ccc(OCC(=O)O)c(F)c2C(C)C)c1F] | 2-(2-fluoro-4-(2-fluoro-4-hydroxy- 3-isopropylbenzyl)-3- isopropylphenoxy)acetic acid |
| 32 | 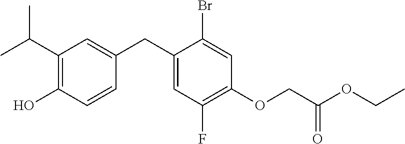 [SMILES: CCOC(=O)COc1cc(Br)c(Cc2ccc(O)c(C(C)C)c2)cc1F] | ethyl 2-(5-bromo-2-fluoro-4-(4- hydroxy-3- isopropylbenzyl)phenoxy)acetate |
| 33 | 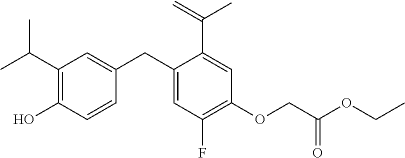 [SMILES: C=C(C)c1cc(OCC(=O)OCC)c(F)cc1Cc1ccc(O)c(C(C)C)c1] | ethyl 2-(2-fluoro-4-(4-hydroxy-3- isopropylbenzyl)-5-(prop-l-en-2- yl)phenoxy)acetate |
| 34 | 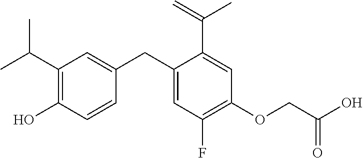 [SMILES: C=C(C)c1cc(OCC(=O)O)c(F)cc1Cc1ccc(O)c(C(C)C)c1] | 2-(2-fluoro-4-(4-hydroxy-3- isopropylbenzyl)-5-(prop-l-en-2- yl)phenoxy)acetic acid |
| 35 | 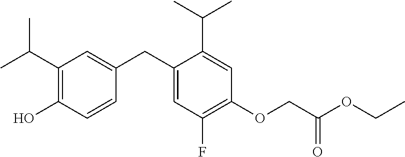 [SMILES: CCOC(=O)COc1cc(C(C)C)c(Cc2ccc(O)c(C(C)C)c2)cc1F] | ethyl 2-(2-fluoro-4-(4-hydroxy-3- isopropylbenzyl)-5- isopropylphenoxy)acetate |
| 36 | 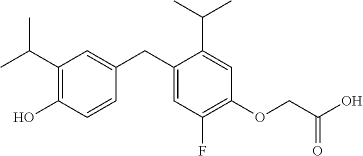 [SMILES: CC(C)c1cc(Cc2cc(F)c(OCC(=O)O)cc2C(C)C)ccc1O] | 2-(2-fluoro-4-(4-hydroxy-3- isopropylbenzyl)-5- isopropylphenoxy)acetic acid |
| 37 | 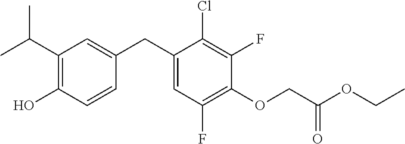 [SMILES: CCOC(=O)COc1c(F)cc(Cc2ccc(O)c(C(C)C)c2)c(Cl)c1F] | ethyl 2-(3-chloro-2,6-difluoro-4-(4- hydroxy-3- isopropylbenzyl)phenoxy)acetate |
| 38 | 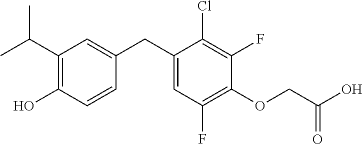 [SMILES: CC(C)c1cc(Cc2cc(F)c(OCC(=O)O)c(F)c2Cl)ccc1O] | 2-(3-chloro-2,6-difluoro-4-(4- hydroxy-3- isopropylbenzyl)phenoxy)acetic acid |
| 39 | 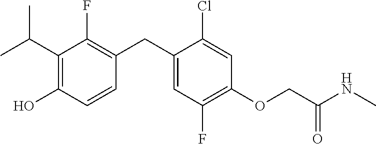 [SMILES: CNC(=O)COc1cc(Cl)c(Cc2ccc(O)c(C(C)C)c2F)cc1F] | 2-(5-chloro-2-fluoro-4-(2-fluoro-4- hydroxy-3- isopropylbenzyl)phenoxy)-N- methylacetamide |
| 40 | 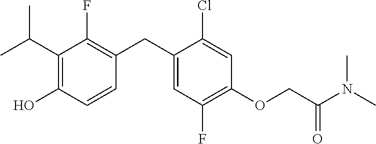 [SMILES: CC(C)c1c(O)ccc(Cc2cc(F)c(OCC(=O)N(C)C)cc2Cl)c1F] | 2-(5-chloro-2-fluoro-4-(2-fluoro-4- hydroxy-3- isopropylbenzyl)phenoxy)-N,N- dimethylacetamide |
| 41 | 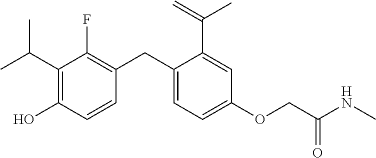 [SMILES: C=C(C)c1cc(OCC(=O)NC)ccc1Cc1ccc(O)c(C(C)C)c1F] | 2-(4-(2-fluoro-4-hydroxy-3- isopropylbenzyl)-3-(prop-1-en-2- yl)phenoxy)-N-methylacetamide |
| 42 | 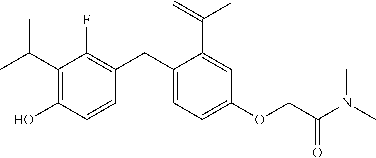 [SMILES: C=C(C)c1cc(OCC(=O)N(C)C)ccc1Cc1ccc(O)c(C(C)C)c1F] | 2-(4-(2-fluoro-4-hydroxy-3- isopropylbenzyl)-3-(prop-1-en-2- yl)phenoxy)-N,N- dimethylacetamide |
| 43 | 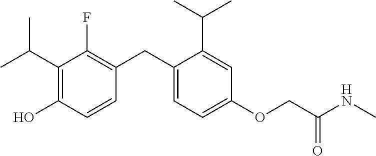 [SMILES: CNC(=O)COc1ccc(Cc2ccc(O)c(C(C)C)c2F)c(C(C)C)c1] | 2-(4-(2-fluoro-4-hydroxy-3- isopropylbenzyl)-3- isopropylphenoxy)-N- methylacetamide |
| 44 | 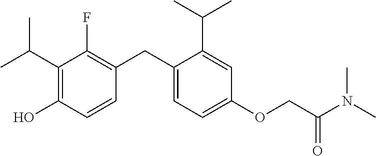 [SMILES: CC(C)c1cc(OCC(=O)N(C)C)ccc1Cc1ccc(O)c(C(C)C)c1F] | isopropylbenzyl)-3- isopropylphenoxy)-N,N- dimethylacetamide |
| 45 | 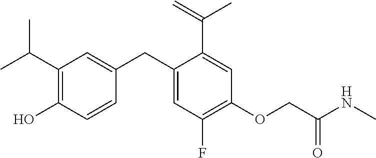 [SMILES: C=C(C)c1cc(OCC(=O)NC)c(F)cc1Cc1ccc(O)c(C(C)C)c1] | 2-(2-fluoro-4-(4-hydroxy-3- isopropylbenzyl)-5-(prop-l-en-2- yl)phenoxy)-N-methylacetamide |
| 46 | 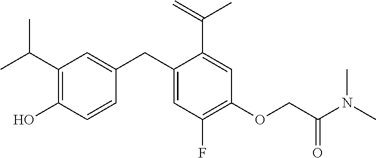 [SMILES: C=C(C)c1cc(OCC(=O)N(C)C)c(F)cc1Cc1ccc(O)c(C(C)C)c1] | 2-(2-fluoro-4-(4-hydroxy-3- isopropylbenzyl)-5-(prop-l-en-2- yl)phenoxy)-N,N- dimethylacetamide |
| 47 | 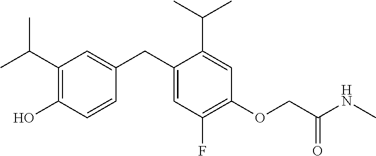 [SMILES: CNC(=O)COc1cc(C(C)C)c(Cc2ccc(O)c(C(C)C)c2)cc1F] | 2-(2-fluoro-4-(4-hydroxy-3- isopropylbenzyl)-5- isopropylphenoxy)-N- methylacetamide |
| 48 | 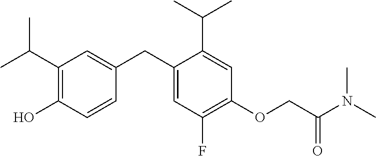 [SMILES: CC(C)c1cc(Cc2cc(F)c(OCC(=O)N(C)C)cc2C(C)C)ccc1O] | 2-(2-fluoro-4-(4-hydroxy-3- isopropylbenzyl)-5- isopropylphenoxy)-N,N- dimethylacetamide |

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure by weight. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out). All compounds with an asterisk (*) adjacent to a tertiary or quaternary carbon are optically active isomers, which may be purified from the respective racemate and/or synthesized by appropriate chiral synthesis.

A “tautomer” refers to each of two or more structural isomers that readily interconvert in equilibrium by migration of an atom or group within the molecule. A tautomer commonly arises from a proton shift from one atom of a molecule to another atom of the same molecule. The present invention includes tautomers of compounds of Formula (I).

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of Formula (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine 19 is longest-lived. Thus, an isotope of a compound having the structure of Formula (I) includes, but not limited to, compounds of Formula (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the invention include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), tromethamine (tris-hydroxymethyl methylamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, βhydroxybutyric, salicylic, galactaric, and galacturonic acid.

Although pharmaceutically unacceptable salts are not generally useful as medicaments, such salts may be useful, for example as intermediates in the synthesis of compounds having the structure of Formula I, for example in their purification by recrystallization.

In certain embodiments, the invention provides a pharmaceutical composition comprising a compound of the invention together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, poly oxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, isotope or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

As used herein, the term “pharmaceutically acceptable carrier” refers to any ingredient other than the disclosed compounds, or a pharmaceutically acceptable isomer, racemate, hydrate, solvate, isotope or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal, or parenteral, including intravenous, subcutaneous and/or intramuscular. In one embodiment, the route of administration is oral.

Dosage forms can be administered once a day, or more than once a day, such as twice or thrice daily. Alternatively, dosage forms can be administered less frequently than daily, such as every other day, or weekly, if found to be advisable by a prescribing physician or drug's prescribing information. Dosing regimens include, for example, dose titration to the extent necessary or useful for the indication to be treated, thus allowing the patient's body to adapt to the treatment, to minimize or avoid unwanted side effects associated with the treatment, and/or to maximize the therapeutic effect of the present compounds. Other dosage forms include delayed or controlled-release forms. Suitable dosage regimens and/or forms include those set out, for example, in the latest edition of the Physicians' Desk Reference, incorporated herein by reference.

In another embodiment, there are provided methods of making a composition of a compound described herein including formulating a compound of the invention with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

In another embodiment, a method of treating a subject having a neurodegenerative disease is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof. In one embodiment, the neurodegenerative disease is a demyelinating disease. In another embodiment, the demyelinating disease is a chronic demyelinating disease. In yet another embodiment, the demyelinating disease is or is associated with a X-linked genetic disorder, leukodystrophy, dementia, tauopathy, or ischaemic stroke. In another embodiment, the demyelinating disease is or is associated with adult Refsum disease, Alexander disease, Alzheimer's disease, Balo concentric sclerosis, Canavan disease, central pontine myelinolysis (CPM), cerebral palsy, cerebrotendineous xanthomatosis, chronic inflammatory demyelinating polyneuropathy (CIDP), Devic's syndrome, diffuse myelinoclastic sclerosis, encephalomyelitis, idiopathic inflammatory demyelinating disease (IIDD), infantile Refsum disease, Krabbe disease, Leber hereditary optic neuropathy, Marburg multiple sclerosis, Marchiafava-Bignami disease, metachromatic leukodystrophy, multifocal motor neuropathy, paraproteinemic demyelinating polyneuropathy, Pelizaeus-Merzbacher disease, peroneal muscular atrophy, progressive multifocal leukoencephalopathy, transverse myelitis, tropical spastic paraparesis, van der Knaap disease, or Zellweger syndrome. In one embodiment, the demyelinating disease is or is associated with multiple sclerosis, MCT8 deficiency, X-linked adrenoleukodystrophy (ALD), amyotrophic lateral sclerosis (ALS), Alzheimer's disease, frontotemporal dementia, or lacunar stroke.

As used herein, the term “neurodegenerative disease” refers to any type of disease that is characterized by the progressive deterioration of the nervous system.

As used herein, the term “demyelinating disease” refers to any disease or medical condition of the nervous system in which myelin is damaged or lost, or in which the growth or development of the myelin sheath is impaired. Demyelination inhibits the conduction of signals in the affected nerves, causing impairment in sensation, movement, cognition, or other functions for which nerves are involved. Demyelinating diseases have a number of different causes and can be hereditary or acquired. In some cases, a demyelinating disease is caused by an infectious agent, an autoimmune response, a toxic agent or traumatic injury. In other cases, the cause of the demyelinating disease is unknown (“idiopathic”) or develops from a combination of factors.

As used herein, the term “leukodystrophy” refers to a group of diseases that affects the growth or development of the myelin sheath.

As used herein, the term “leukoencephalopathy” refers to any of a group of diseases affecting the white substance of the brain; can refer specifically to several diseases including for example, “leukoencephalopathy with vanishing white matter” and “toxic leukoencephalopathy.” Leukoencephalopathies are leukodystrophy-like diseases.

As used herein, the term “tauopathy” refers to tau-related disorders or conditions, e.g., Alzheimer's Disease (AD), Progressive Supranuclear Palsy (PSP), Corticobasal Degeneration (CBD), Pick's Disease (PiD), Argyrophilic grain disease (AGD), Frontotemporal dementia and Parkinsonism associated with chromosome 17 (FTDP-17), Parkinson's disease, stroke, traumatic brain injury, mild cognitive impairment and the like.

As used herein, the terms “multiple sclerosis” and “MS” refer to a slowly progressive CNS disease characterized by disseminated patches of demyelination in the brain and spinal cord, resulting in multiple and varied neurological symptoms and signs, usually with remissions and exacerbation. The cause of MS is unknown but an immunological abnormality is suspected. An increased family incidence suggests genetic susceptibility, and women are somewhat more often affected than men. The symptoms of MS include weakness, lack of coordination, paresthesias, speech disturbances, and visual disturbances, most commonly double vision. More specific signs and symptoms depend on the location of the lesions and the severity and destructiveness of the inflammatory and sclerotic processes. Relapsing-remitting multiple sclerosis (RRMS) is a clinical course of MS that is characterized by clearly defined, acute attacks with full or partial recovery and no disease progression between attacks. Secondary-progressive multiple sclerosis (SPMS) is a clinical course of MS that initially is relapsing-remitting, and then becomes progressive at a variable rate, possibly with an occasional relapse and minor remission. Primary-progressive multiple sclerosis (PPMS) presents initially in the progressive form. A clinically isolated syndrome is the first neurologic episode, which is caused by inflammation/demyelination at one or more sites in the CNS. Progressive-relapsing multiple sclerosis (PRMS) is a rare form of MS (˜5%) characterized by a steadily worsening disease state from onset, with acute relapses but no remissions.

In yet another embodiment, a method of treating a subject having a X-linked genetic disorder is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof. In one embodiment, the X-linked genetic disorder is MCT8 deficiency or X-linked adrenoleukodystrophy (ALD).

In another embodiment, a method of treating a subject having a leukodystrophy is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof. In one embodiment, the leukodystrophy is adrenoleukodystrophy (ALD), adrenomyeloneuropathy (ANN), cerebral form of adrenoleukodystrophy (cALD), metachromatic leukodystrophy (MLD), Canavan's disease, or Krabbe disease (globoid leukodystrophy). As used herein, the term “adrenomyeloneuropathy” or “ANN” refers to an adult variant of X-linked adrenoleukodystrophy, characterized by ABCD1 gene mutation, that results in impaired peroxisome function with accumulation of very long chain fatty acids (VLCFA) and demyelination.

In one embodiment, a method of treating a subject having a tauopathy is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof. In one embodiment, the tauopathy is Alzheimer's disease, frontotemporal dementia, primary age-related tauopathy (PART), Pick's disease, or frontotemporal dementia and parkinsonism linked to chromosome 17 (FTDP-17).

In yet another embodiment, a method of treating a subject having an ischaemic stroke is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof. In one embodiment, the ischaemic stroke is lacunar stroke (also called “lacunar infarct”). In another embodiment, the present method is used to treat a subject suffering from a lacunar stroke syndrome (LACS).

In another embodiment, a method of treating a subject having adult Refsum disease, infantile Refsum disease, Alexander disease, Alzheimer's disease, balo concentric sclerosis, Canavan disease, central pontine myelinolysis (CPM), cerebral palsy, cerebrotendineous xanthomatosis, chronic inflammatory demyelinating polyneuropathy (CIDP), Devic's syndrome, diffuse myelinoclastic sclerosis, encephalomyelitis, idiopathic inflammatory demyelinating disease (IIDD), Krabbe disease, Leber hereditary optic neuropathy, leukodystrophy, Marburg multiple sclerosis, Marchiafava-Bignami disease, metachromatic leukodystrophy (MLD), multifocal motor neuropathy (MMN), multiple sclerosis (MS), paraproteinemic demyelinating polyneuropathy, Pelizaeus-Merzbacher disease (PMD), progressive multifocal leukoencephaalopathy (PML), tropical spastic paraparesis (TSP), X-linked adrenoleukodystrophy (X-ALD, ALO, or X-linked ALO), or Zellweger syndrome is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In one embodiment, the demyelinating disease is multiple sclerosis. In another embodiment, the demyelinating disease is X-linked adrenoleukodystrophy (ALD).

In another embodiment, a method of treating a subject having an amyotrophic lateral sclerosis (ALS) disease is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof. In one embodiment, the ALS is sporadic or familial ALS, or ALS with Superoxide dismutase-1 mutation.

In one embodiment, a method of treating a subject having a medical condition associated with increased activity of TGF-β is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof. In one embodiment, the medical condition associated with increased activity of TGF-β is a fibrotic disease. In another embodiment, the fibrotic disease is or is associated with nonalcoholic steatohepatitis (NASH), idiopathic pulmonary fibrosis (IPF), systemic scleroderma, or Alport syndrome. As used herein, the term “Alport syndrome” refers to a hereditary disorder caused by mutations in the a3a4a5(IV) collagen network genes resulting in structural defects in the glomerular basement membrane (GBM) early during development leading subsequently to the breakdown of the filtration barrier, development of renal fibrosis and kidney failure.

As used herein, the term “fibrotic disease” refers to a condition, disease or disorder that is amenable to treatment by administration of a compound having anti-fibrotic activity. Fibrotic diseases include, but are not limited to, pulmonary fibrosis, including idiopathic pulmonary fibrosis (IPF) and pulmonary fibrosis from a known etiology, liver fibrosis, and renal-fibrosis. Other exemplary fibrotic diseases include musculoskeletal fibrosis, cardiac fibrosis, post-surgical adhesions, scleroderma, glaucoma, and skin lesions such as keloids.

In another embodiment, a method of treating a subject having NASH, NAFLD, NAFLD with hyperlipidemia, alcoholic liver disease/alcoholic steatohepatitis, liver fibrosis associated with viral infection (HBV, HCV), fibrosis associated with cholestatic diseases (primary biliary cholangitis, primary sclerosing cholangitis), (familial) hypercholesterolemia, dyslipidemia, genetic lipid disorders, cirrhosis, alcohol-induced fibrosis, hemochromatosis, glycogen storage diseases, alpha-1 antitrypsin deficiency, autoimmune hepatitis, Wilson's disease, Crigler-Najjar Syndrome, lysosomal acid lipase deficiency, liver disease in cystic fibrosis is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having Alport syndrome, diabetic nephropathy, FSGS, fibrosis associated with IgA nephropathy, chronic kidney diseases (CKD), post AKI, HIV associated CKD, chemotherapy induced CKD, CKD associated with nephrotoxic agents, nephrogenic systemic fibrosis, tubulointerstitial fibrosis, glomerulosclerosis, or polycystic kidney disease (PKD) is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having IPF, ILD, pulmonary fibrosis, pulmonary fibrosis associated with autoimmune diseases like rheumatoid arthritis, scleroderma or Sjogren's syndrome, asthma-related pulmonary fibrosis, COPD, asbestos or silica induced PF, silicosis, respiratory bronchiolitis, Idiopathic interstitial pneumonias (IIP), Idiopathic nonspecific interstitial pneumonia, Respiratory bronchiolitis-interstitial lung disease, desquamative interstitial pneumonia, acute interstitial pneumonia, Rare IIPs: Idiopathic lymphoid interstitial pneumonia, idiopathic pleuroparenchymal fibroelastosis, unclassifiable idiopathic interstitial pneumonias, hypersensitivity pneumonitis, radiation-induced lung injury, progressive massive fibrosis—pneumoconiosis, bronchiectasis, byssinosis, chronic respiratory disease, chronic obstructive pulmonary disease (COPD), emphysema, pulmonary arterial hypertension (PAH), or Cystic fibrosis is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having scleroderma/systemic sclerosis, graft versus host disease, hypertrophic scars, keloids, nephrogenic systemic fibrosis, Porphyria cutanea tarda, restrictive dermopathy, Dupuytren's contracture, dermal fibrosis, nephrogenic systemic fibrosis/nephrogenic fibrosing dermopathy, mixed connective tissue disease, scleromyxedema, eosinophilic fasciitis, fibrosis caused by exposure to chemicals or physical agents. GvHD induced fibrosis, Scleredema adultorum, Lipodermatosclerosis, or Progeroid disorders (progeria, acrogeria, Werner's syndrome) is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having atrial fibrosis, endomyocardial fibrosis, cardiac fibrosis, atherosclerosis, restenosis, or arthrofibrosis is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having mediastinal fibrosis, myelofibrosis, post-polycythermia vera myelofibrosis, or post essential thrombocythemia is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having Crohn's disease, retroperitoneal fibrosis, intestinal fibrosis, fibrosis in inflammatory bowel disease, ulcerative colitis, GI fibrosis due to cystic fibrosis, or pancreatic fibrosis due to pancreatitis is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having endometrial fibrosis, uterine fibroids, or Peyronie's disease is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having macular degeneration, diabetic retinopathy, retinal fibrovascular diseases, or vitreal retinopathy is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

In another embodiment, a method of treating a subject having scarring associated with trauma (surgical complications, chemotherapeutics drug-induced fibrosis, radiation induced fibrosis) is provided, the method comprising administering to the subject a pharmaceutically effective amount of a compound having the structure of Formula (I) or (I′) or pharmaceutically acceptable isomer, racemate, hydrate, solvate, isotope or salt thereof, or a pharmaceutical composition thereof.

As used herein, the term “administration” refers to providing a compound, a prodrug of a compound, or a pharmaceutical composition comprising the compound or prodrug as described herein. The compound or composition can be administered by another person to the subject or it can be self-administered by the subject. Non-limiting examples of routes of administration are oral, parenteral (e.g., intravenous), or topical.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human). A subject to be treated according to the methods described herein may be one who has been diagnosed with a neurodegenerative disease involving demyelination, insufficient myelination, or underdevelopment of a myelin sheath, e.g., a subject diagnosed with multiple sclerosis or cerebral palsy, or one at risk of developing the condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

As used herein, the terms “chronic” refers to a medical disorder or condition that persists overtime or is frequently recurring.

Compounds having the structure of Formulas (I)-(VIII) and (I′)-(VIII′) can be synthesized using standard synthetic techniques known to those skilled in the art. For example, compounds of the present invention can be synthesized using appropriately modified synthetic procedures set forth in Schemes 1-20 below.

To this end, the reactions, processes, and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary.

Generally, suitable solvents are protic or aprotic solvents which are substantially non-reactive with the reactants, the intermediates or products at the temperatures at which the reactions are carried out (i.e., temperatures which may range from the freezing to boiling temperatures, or higher if reactions are run in sealed vessels). A given reaction may be carried out in one solvent or a mixture of more than one solvent. Depending on the particular reaction, suitable solvents for a particular work-up following the reaction may be employed.

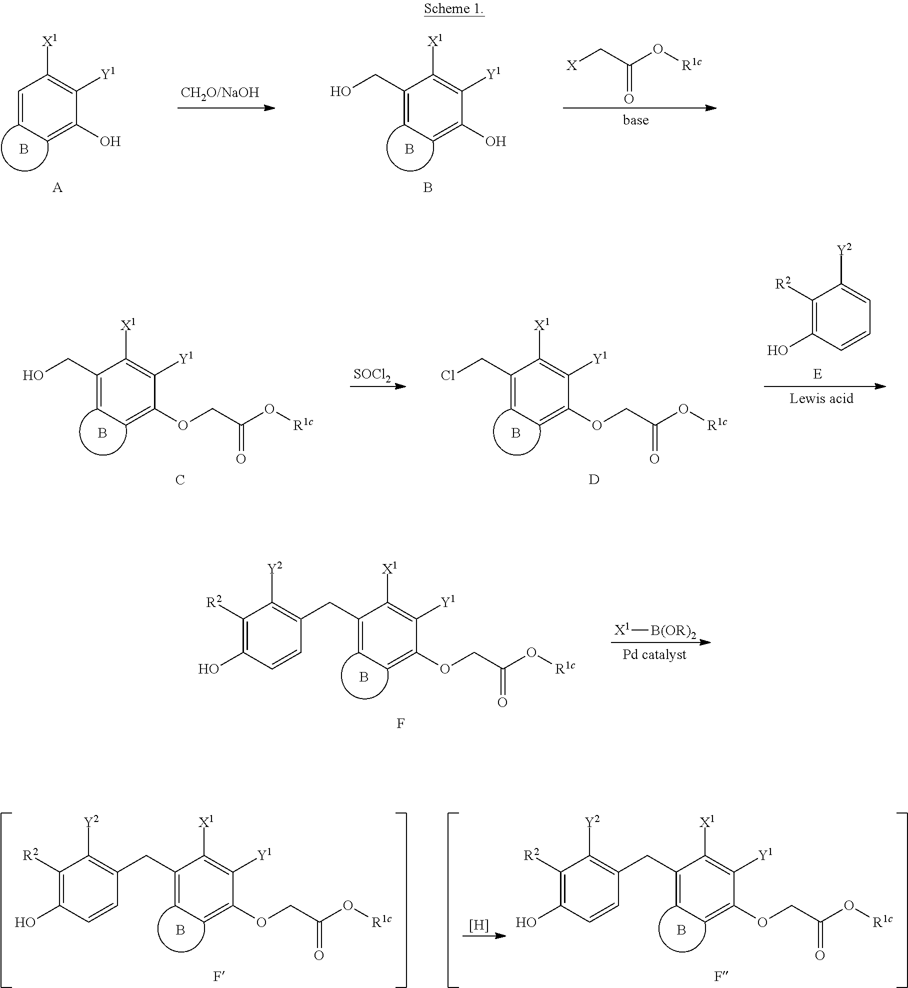

Compounds of the present invention can be prepared according to Scheme 1. Referring to Scheme 1, mono- or di-substituted or unsubstituted bicyclic phenol (A) (for example, 4-indanol or 6-methyl-4-indanol or the like) is reacted with a formaldehyde equivalent (for example, aqueous formaldehyde or paraformaldehyde or dimethoxymethane or the like) to give a hydroxymethyl derivative (B), which is subsequently reacted with an activated acetate moiety (for example ethyl chloroacetate or methyl bromoacetate or the like) in the presence of base, selectively at the phenolic oxygen, to provide intermediate (C). The hydroxymethyl group is activated (for example, through reaction with thionyl chloride or oxalyl chloride or p-toluenesulfonylchloride or the like) to give a chloromethyl derivative (D) (or the corresponding tosylate, or mesylate, or bromomethyl analog, or the like), which is condensed with a 2-substituted phenol (E) in the presence of a Lewis acid (like zinc chloride, or aluminum chloride or the like) to give an ester (F). Alternatively, alcohol intermediate C can be reacted directly with phenol (E) in the presence of a protic acid (for example sulfuric acid or the like), or a Lewis acid (for example boron trifluoride etherate or the like). In cases in which X¹ is a bromide or iodide, intermediate F can be reacted under Suzuki coupling conditions (for example using a boronic acid or boronate reagent or the like in the presence of a Palladium catalyst like Pd(OAc)₂ or Pd(dppf)Cl₂ or the like), to produce alkyl, alkenyl, or alkynyl products (F′). In cases where X¹ is an alkene or alkyne, subsequent hydrogenation (for example using Pd—C catalyst under a hydrogen atmosphere) can provide the corresponding alkyl-substituted (F″).

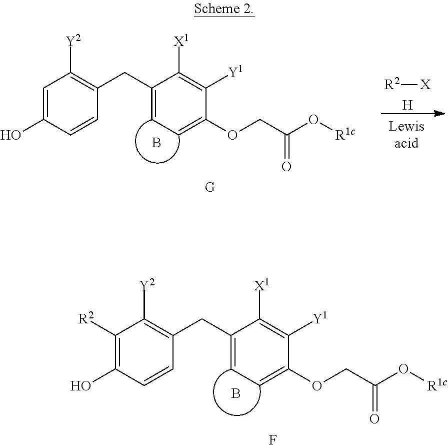

Compounds of the present invention can be prepared according to Scheme 2. Referring to Scheme 2, phenol G (G=F, where R²═H; prepared according to Scheme 1) is reacted with an alcohol or reactive halide (H) (for example p-fluorobenzyl chloride or 1-(1-chloroethyl)-4-fluoro-benzene or 2,4-difluorobenzyl alcohol or the like) in the presence of a Lewis acid (like zinc chloride or aluminum chloride or boron trifluoride etherate or the like) to give a 3′-alkylated product like ester (F) of the present invention.

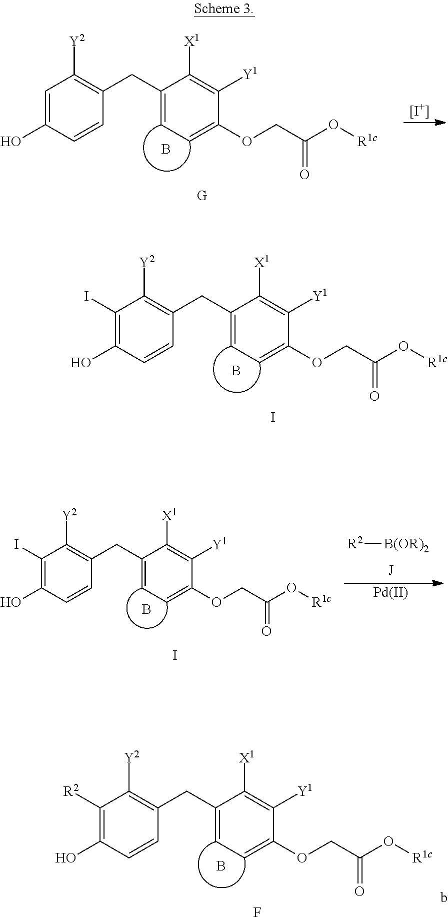

Compounds of the present invention can be prepared according to Scheme 3. Referring to Scheme 3, ortho-iodination of phenol G (for example using N-iodosuccinimide or solid iodine or the like) provides key intermediate I. To prepare compounds of the present invention, I is reacted with a boronic acid or boronate (J) under various Suzuki conditions (for example in the presence of a Palladium catalyst like Pd(OAc)₂ or Pd(dppf)Cl₂ or the like) to provide ester (F) of the present invention.

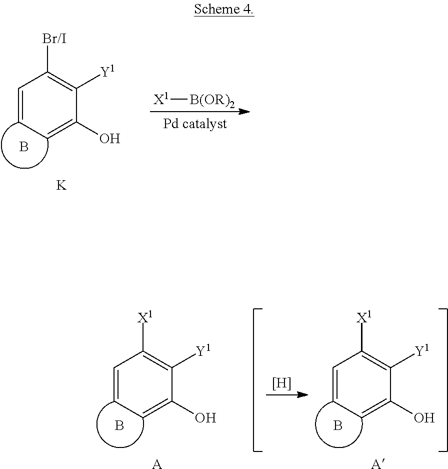

Phenols (A) of the present invention may be commercially available, or may be prepared according to Scheme 4. Referring to Scheme 4, a mono- or di-substituted bicyclic phenol (K) having one substituent as bromine or iodine may be reacted under Suzuki coupling conditions (for example using a boronic acid or boronate reagent or the like, in the presence of a Palladium catalyst like Pd(OAc)₂ or Pd(dppf)Cl₂ or the like) to produce alkyl, alkenyl, or alkynyl products (A). In the case where X¹ is an alkene or alkyne, subsequent hydrogenation (for example using Pd—C catalyst under a hydrogen atmosphere or the like) can provide the corresponding alkyl-substituted phenol (A′).

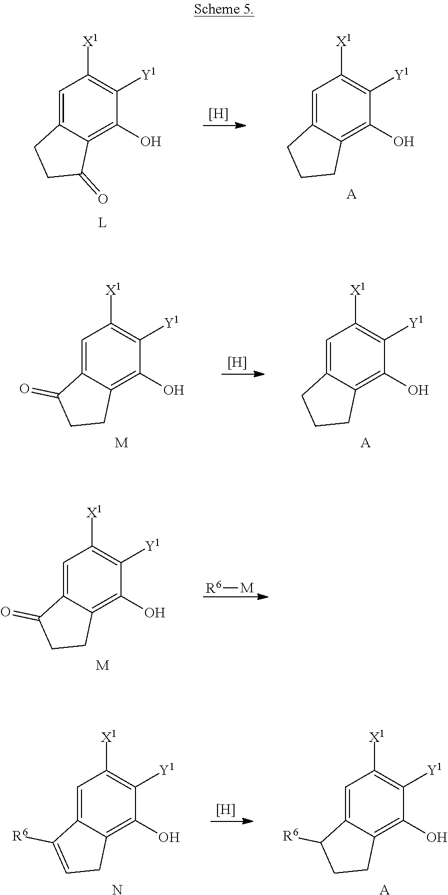

Alternative approaches to prepare phenols (A) of the present invention are described in Scheme 5. Referring to Scheme 5, mono-, di- or unsubstituted 7-hydroxy-1-indanone (L) (for example 5-methyl-7-hydroxy-1-indanone or the like) is deoxygenated (for example with zinc/hydrochloric acid or the like) to give the corresponding bicyclic phenol (A). Alternatively, mono-, di- or unsubstituted 4-hydroxy-1-indanone (M) (for example 4-hydroxy-1-indanone or the like) is deoxygenated (for example with zinc/hydrochloric acid or the like) to give the corresponding bicyclic phenol (A). Alternatively, mono-, di- or unsubstituted 4-hydroxy-1-indanone (M) (for example 4-hydroxy-1-indanone or the like) can be treated with Grignard reagent (for example methylmagnesium chloride or the like) to give alkene (N), and then hydrogenated (for example using Pd—C catalyst under a hydrogen atmosphere or the like) to provide the corresponding alkyl-substituted phenol (A).

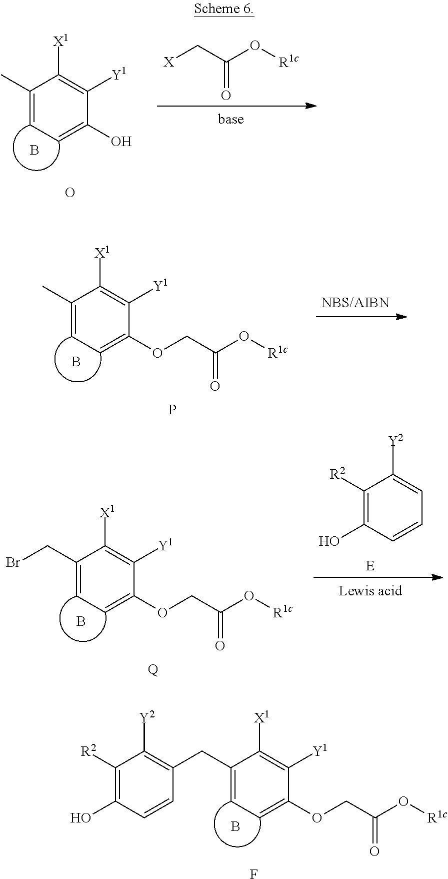

Compounds of the present invention can be prepared according to Scheme 6. Referring to Scheme 6, mono- di- or unsubstituted bicyclic phenol (O) is reacted with an activated acetate moiety (for example ethyl chloroacetate or methyl bromoacetate or the like) in the presence of base, to provide intermediate P. Intermediate P can be brominated (for example using N-bromosuccinimide (NBS) in the presence of a radical initiator azobisisobutyronitrile (AIBN) or the like) to provide intermediate Q, which is condensed with phenol (E) in the presence of a Lewis acid (such as zinc chloride or aluminum chloride or the like) to give an ester (F) of the present invention.

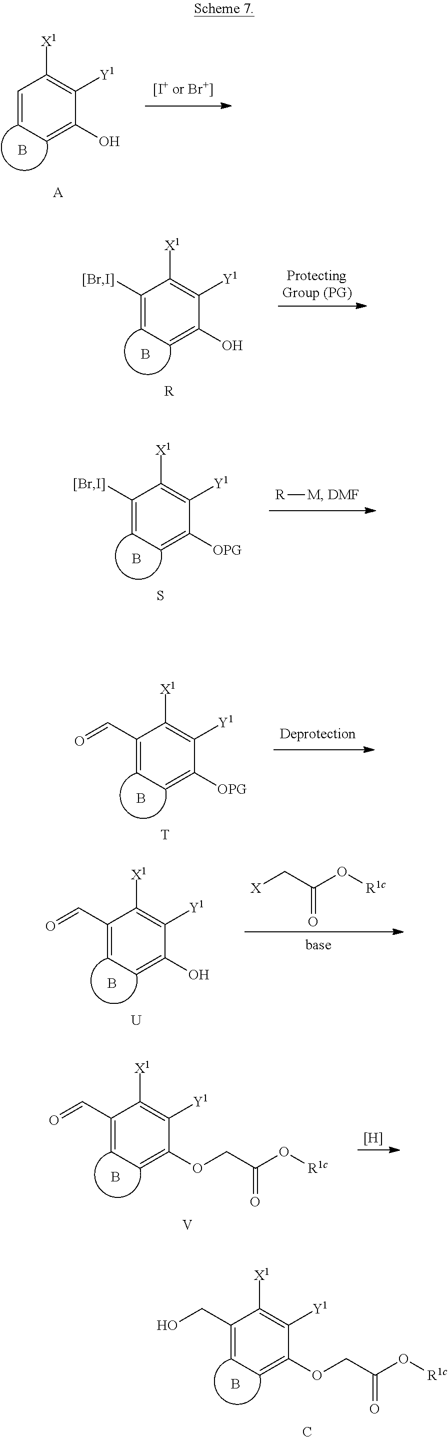

Hydroxy methyl derivatives (C) of the present invention can be prepared according to Scheme 7. Referring to Scheme 7, a mono-, di- or unsubstituted substituted bicyclic phenol (A) is halogenated (for example with N-iodosuccinimide (NIS) or the like) to give halo-substituted phenol (R). Intermediate R is protected (for example as the tri-isopropyl silyl ether using tri-isopropyl silyl triflate or the like) to give intermediate S. Bromo- or iodo-substituted intermediate S may be metallated (for example using isopropylmagnesium bromide or n-butyllithium or the like), then quenched with DMF to give aldehyde (T), which is subsequently deprotected (for example by treatment with a fluoride source like tetra-n-butylammonium fluoride or the like) to give phenol (U). Intermediate U can be reacted with an activated acetate moiety (for example ethyl chloroacetate or methyl bromoacetate or the like) in the presence of base, to provide aldehyde (V). Aldehyde intermediate V can be reduced (for example with sodium borohydride or the like) give benzylic alcohol intermediate C.

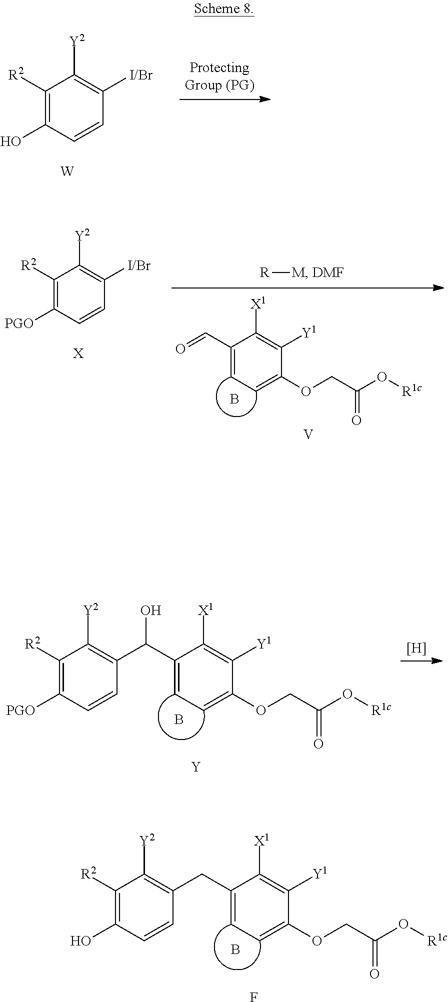

Compounds of the present invention can be prepared according to Scheme 8. Referring to Scheme 8, 4-halophenol intermediate W is masked with an appropriate protecting group (for example treatment with methoxymethyl chloride to provide MOM-protection) to give intermediate X. Intermediate X is metallated (for example using isopropylmagnesium bromide or n-butyllithium or the like) and condensed with aldehyde intermediate V (for example obtained commercially of synthesized according to Scheme 7 or the like), to give alcohol (Y). Intermediate Y is deoxygenated under hydrogenolysis conditions that promote concomitant deprotection (for example by treatment with trifluoroacetic acid or the like and triethylsilane or the like) to provide intermediate F of the present invention. Deoxygenation and deprotection of Intermediate Y can also proceed in stepwise manner.

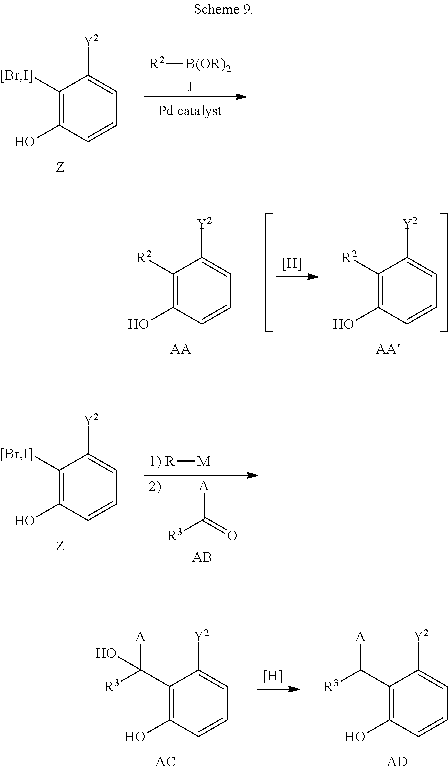

Substituted phenols (E) as employed in Scheme 1 may be prepared as indicated in Scheme 9. Referring to Scheme 9, a 2-halophenol (Z) (such as 2-bromophenol or 2-bromo-3-fluorophenol or the like) may be condensed with a boronic acid or ester (J) under Suzuki conditions (for example in the presence of a Palladium catalyst like Pd(OAc)₂ or Pd(dppf)Cl₂ or the like) to give 2-substituted phenol (AA). In the case where the R² group is an alkene or alkyne, subsequent hydrogenation (for example using Pd—C catalyst under a hydrogen atmosphere or the like) can provide the corresponding alkyl-substituted phenol (AA′). Alternatively, a 2-halophenol (AA) (for example 2-bromophenol or 2-bromo-3-fluorophenol or the like) may be metallated (for example using isopropylmagnesium bromide or n-butyllithium or the like) then condensed with an aldehyde or ketone (AB), to give benzylic alcohol intermediate AC. Alcohol (AC) can be deoxygenated under hydrogenolysis conditions (for example hydrogen gas in the presence of a palladium or platinum catalyst or the like, or under reductive-deoxygenation conditions in the presence of a reducing agent triethylsilane or the like or in the presence of an acid like TFA or the like) to provide substituted phenol (E).

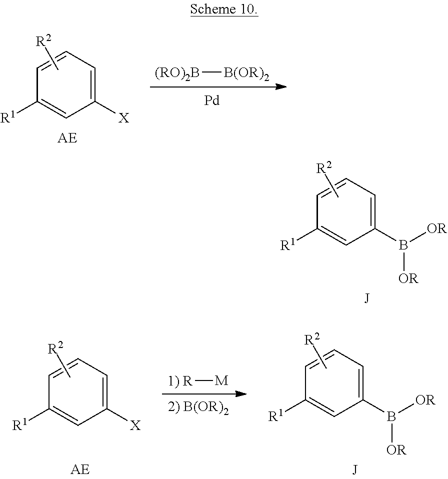

Arylboronic acids or esters (J) as employed in Scheme 3 and 9 may be sourced commercially, or may be prepared as described in Scheme 10. Referring to Scheme 10, substituted aryl halide (AE) may be reacted with di(pinacolato)diboron or a similar reagent, using a palladium catalyst or the like, to give (J). Alternatively, (AE) may be metallated (for example using isopropylmagnesium bromide or n-butyllithium or the like), then reacted with a trialkoxyborate or the like, to provide (J).

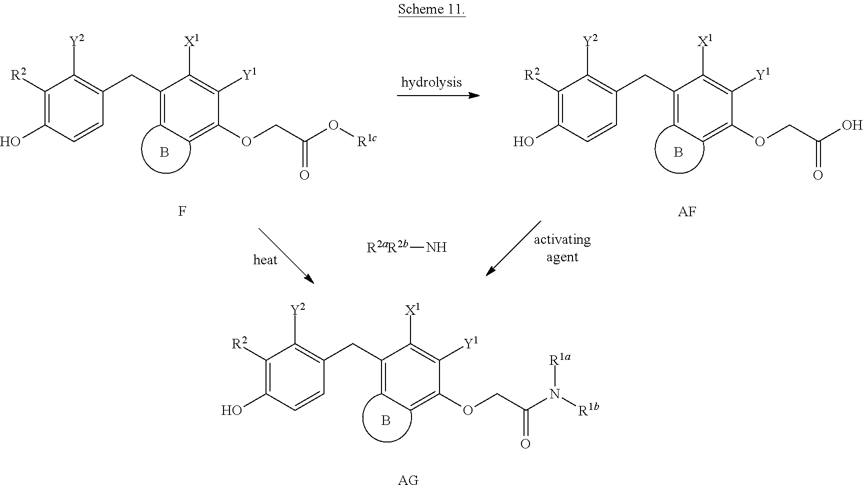

As shown in Scheme 11, hydrolysis of the ester group of intermediate F (for example using aqueous sodium hydroxide (if R^{1c} is methyl) or TFA (if R^{1c} is t-butyl)) provides acid (AF) of the present invention. If desired, acid (AF) can be converted to an amide (AG) by condensing with the corresponding amine (for example methylamine or propylamine or 2-sulfonylethylamine or the like) in the presence of a coupling agent (for example DDC or EDCI or the like), or by forming an activated intermediate (for example the corresponding acid chloride busing thionyl chloride or the like). Alternatively, if desired, either esters (F), or acids (AF) may be heated with an amine R^{1a}R^{1b}NH (for example methylamine or propylamine or 2-sulfonylethylamine or the like) to give amides (AG) of the present invention.

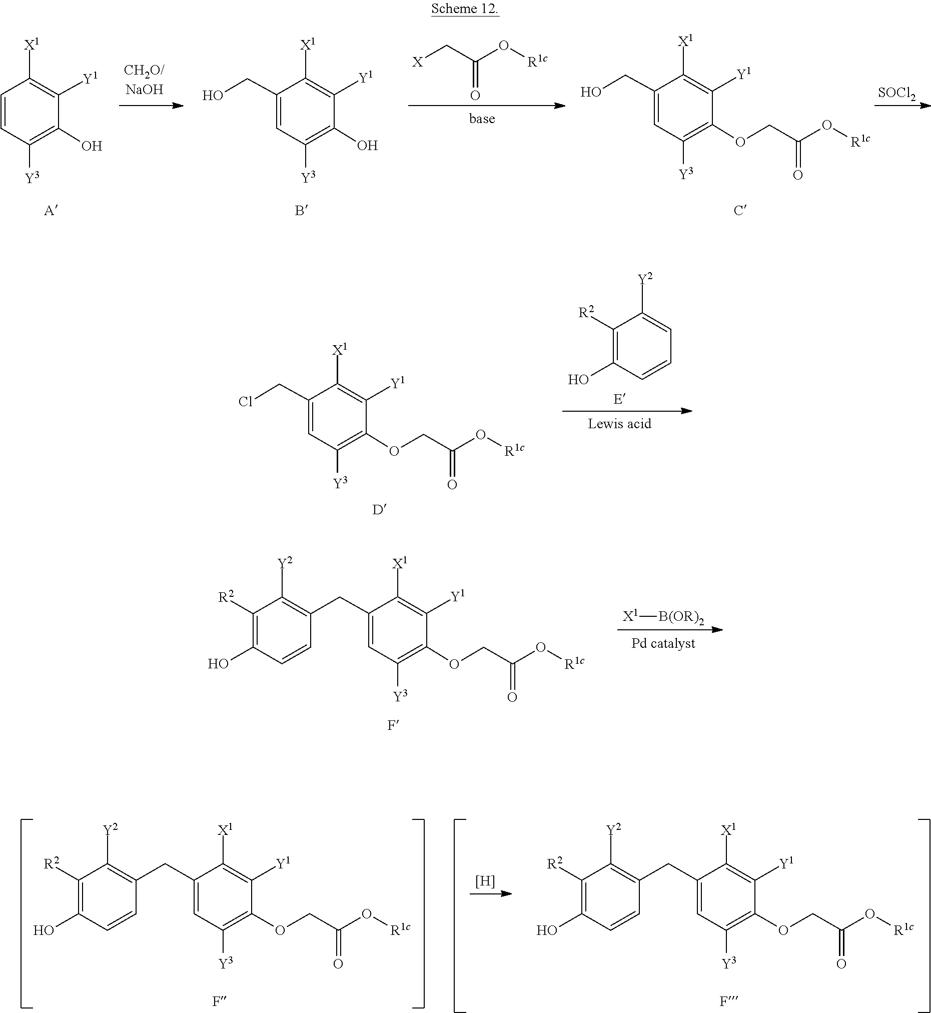

Compounds of the present invention can be prepared according to Scheme 12. Referring to Scheme 1, a mono-, di- or tri-substituted phenol (A′) (for example, 3-chlorophenol or 2-fluoro-5-chlorophenol, or the like) is reacted with a formaldehyde equivalent (for example, aqueous formaldehyde or paraformaldehyde or dimethoxymethane or the like) to give a hydroxymethyl derivative (B′), which is subsequently reacted with an activated acetate moiety (for example ethyl chloroacetate or methyl bromoacetate or the like) in the presence of base, selectively at the phenolic oxygen, to provide intermediate (C′). The hydroxymethyl group is activated (for example, through reaction with thionyl chloride or oxalyl chloride or p-toluenesulfonylchloride or the like) to give a chloromethyl derivative (D′) (or the corresponding tosylate, or mesylate, or bromomethyl analog, or the like), which is condensed with a 2-substituted phenol (E′) in the presence of a Lewis acid (like zinc chloride, or aluminum chloride, or the like) to give an ester (F′). Alternatively, intermediate alcohol (C′) can be reacted directly with phenol (E′) in the presence of a protic acid (for example using sulfuric acid or the like), or a Lewis acid (for example boron trifluoride etherate or the like). In cases in which X¹ is a bromide or iodide, F′ can be reacted under Suzuki coupling conditions (for example using a boronic acid or boronate reagent or the like in the presence of a Palladium catalyst like Pd(OAc)₂ or Pd(dppf)Cl₂ or the like), to produce alkyl, alkenyl, or alkynyl products (F″). In cases where X¹ is an alkene or alkyne, subsequent hydrogenation (for example using Pd—C catalyst under a hydrogen atmosphere) can provide the corresponding alkyl-substituted (F′″).

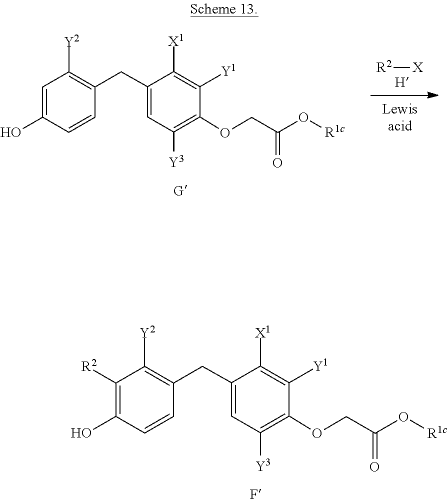

Compounds of the present invention can be prepared according to Scheme 13. Referring to Scheme 2, phenol G′ (G′=F′, R¹═H; prepared according to Scheme 1) is reacted with an alcohol or reactive halide H′ (for example p-fluorobenzyl chloride or 1-(1-chloroethyl)-4-fluoro-benzene or 2,4-difluorobenzyl alcohol or the like) in the presence of a Lewis acid (like Zinc chloride or Aluminum chloride or boron trifluoride etherate or the like) to give a 3′-alkylated product like ester F′.

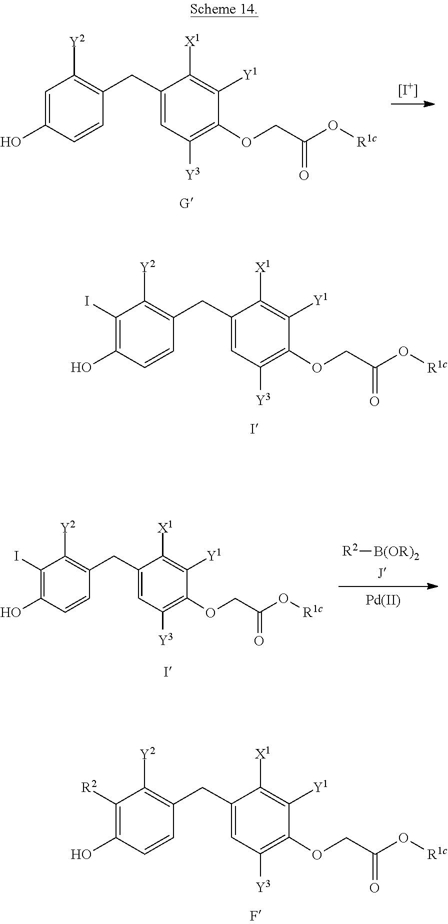

Compounds of the present invention can be prepared according to Scheme 14.

Referring to Scheme 3, ortho-iodination of phenol G′, (for example using N-iodosuccinimide or solid iodine or the like) provides key intermediate I′. To prepare compounds of the present invention, I′ is reacted with a boronic acid (or boronate) J′ under various Suzuki conditions Suzuki (for example in the presence of a Palladium catalyst like Pd(OAc)₂ or Pd(dppf)Cl₂ or the like) to provide esters F′ of the present invention.

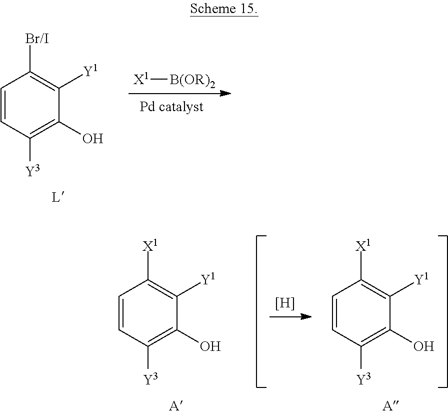

Phenols (A′) of the present invention may be commercially available, or may be prepared according to Scheme 15. Referring to Scheme 15, mono-, di- or tri-substituted phenols (L′) having one substituent as bromine or iodine may be reacted under Suzuki coupling conditions (for example using a boronic acid or boronate reagent or the like, in the presence of a Palladium catalyst like Pd(OAc)₂ or Pd(dppf)Cl₂ or the like) to produce alkyl, alkenyl, or alkynyl products (A′). In the case where X¹ is an alkene or alkyne, subsequent hydrogenation (for example using Pd—C catalyst under a hydrogen atmosphere) can provide the corresponding alkyl-substituted (A″).

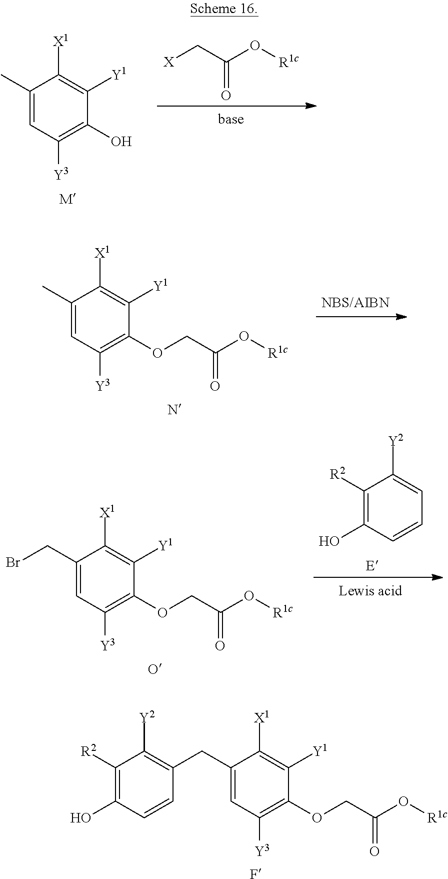

Compounds of the present invention can be prepared according to Scheme 5. Referring to Scheme 5, mono- di- or tri-substituted p-cresol (M′) is reacted with an activated acetate moiety (for example ethyl chloroacetate or methyl bromoacetate or the like) in the presence of base, to provide intermediate N′. Bromination (for example using N-bromosuccinimide (NBS) and radical initiator azobisisobutyronitrile (AIBN) or the like) provides intermediate O′, which is condensed with a 2-substituted phenol (E′) in the presence of a Lewis acid (such as zinc chloride, or aluminum chloride, or the like) to give an ester (F′).

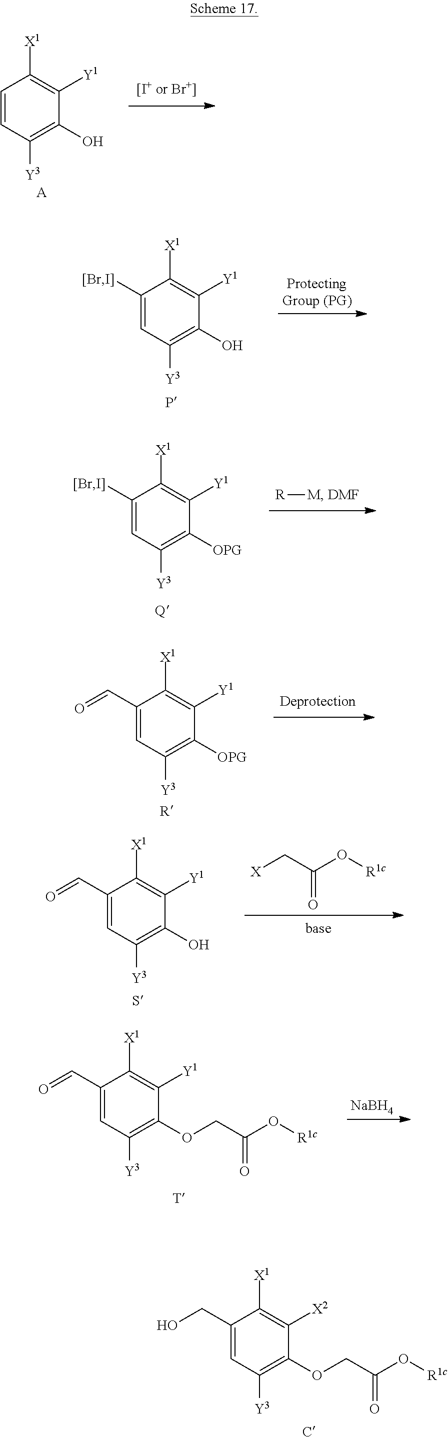

Hydroxy methyl derivative (C′) of the present invention can be prepared according to Scheme 17. Referring to Scheme 6, a mono-, di- or tri-substituted phenol (A′) is halogenation (for example with N-iodosuccinimide (NIS) or the like) to give phenol P′. Intermediate P′ is protected (for example as the tri-isopropyl silyl ether using tri-isopropyl silyl triflate or the like) to give intermediate Q′. Bromo- or iodo-substituted intermediate Q′ may be metallated (for example using isopropylmagnesium bromide or n-butyllithium or the like), then quenched with DMF to give aldehyde (R′), which is subsequently deprotected (for example by treatment with a fluoride source like tetra-n-butylammonium fluoride or the like) to give intermediate S.

Aldehyde intermediate S can be reacted with an activated acetate moiety (for example ethyl chloroacetate or methyl bromoacetate or the like) in the presence of base, to provide aldehyde (T′). Aldehyde intermediate T′ can be reduced (for example with sodium borohydride or the like) give benzylic alcohol intermediate (C′).

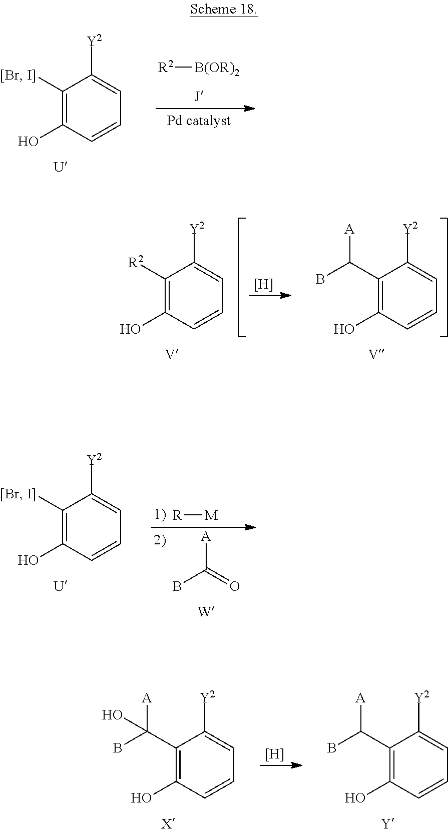

Substituted phenol (E′) as employed in Scheme 1 may be prepared as indicated in Scheme 7 for phenols (V′), (V″) and (Y′). Referring to Scheme 7, a 2-halophenol (U′) (such as 2-bromophenol or 2-bromo-3-fluorophenol or the like) may be condensed with a boronic acid or ester (J′) under Suzuki conditions (for example in the presence of a Palladium catalyst like Pd(OAc)₂ or Pd(dppf)Cl₂ or the like) to give 2-substituted phenol (V′). In the case where the R¹ group is an alkene or alkyne, subsequent hydrogenation (for example using Pd—C catalyst under a hydrogen atmosphere or the like) can provide the corresponding alkyl-substituted phenol (V″).

Alternatively, a 2-halophenol (U′) (for example 2-bromophenol or 2-bromo-3-fluorophenol or the like) may be metallated (for example using isopropylmagnesium bromide or n-butyllithium or the like) then condensed with an aldehyde or ketone (X′), to give benzylic alcohol intermediate (X′). Deoxygenation of (X′) under hydrogenolysis conditions (for example hydrogen gas in the presence of a palladium or platinum catalyst or the like, or under reductive-deoxygenation conditions in the presence of a reducing agent triethylsilane or the like or in the presence of an acid like TFA or the like produces substituted phenol (Y′).

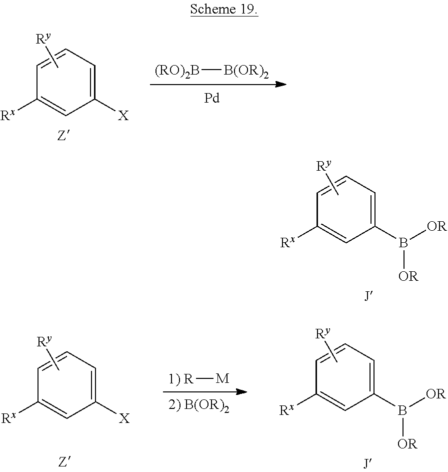

Arylboronic acids or esters (J′) as employed in Scheme 14 and 18 may be sourced commercially, or may be prepared as described in Scheme 19. Referring to Scheme 19, substituted aryl halide (Z′) may be reacted with di(pinacolato)diboron or a similar reagent, using a palladium catalyst or the like, to give (J′). Alternatively (Z′) may be metallated (for example using isopropylmagnesium bromide or n-butyllithium or the like), then reacted with a trialkoxyborate or the like, to provide (J′).

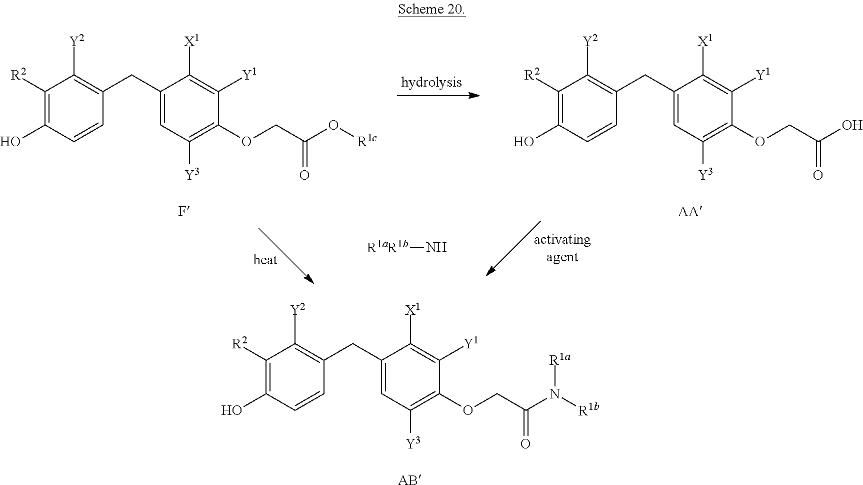

As shown in Scheme 20, hydrolysis of the ester group of intermediate F′ (for example using aqueous sodium hydroxide (if R^{1c} is methyl) or TFA (if R¹ is t-butyl)) provides acid (AA′) of the present invention. If desired, acid (AA′) can be converted to an amide (AB′) by condensing with the corresponding amine (for example methylamine or propylamine or 2-sulfonylethylamine or the like) in the presence of a coupling agent (for example DDC or EDCI or the like), or by forming an activated intermediate (for example the corresponding acid chloride busing thionyl chloride or the like). Alternatively, if desired, either esters (F′), or acids (AA′) may be heated with an amine R^{1a}R^{1b}NH, for example methylamine or propylamine or 2-sulfonylethylamine or the like, to give amides (AB′) of the present invention.

### EXAMPLES

The invention is further illustrated by the following examples. The examples below are non-limiting are merely representative of various aspects of the invention. Solid and dotted wedges within the structures herein disclosed illustrate relative stereochemistry, with absolute stereochemistry depicted only when specifically stated or delineated.

### General Methods

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent.

In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC (normal-phase or reversed-phase) using methods as described. Preparative HPLC purification by reverse phase HPLC was performed using gradients of acetonitrile in aqueous TFA or an equivalent HPLC system such as Methanol in aqueous ammonium acetate.

Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention which is sufficiently acidic, an ammonium salt. A salt of this type can either be transformed into its free base or free acid form, respectively, by various methods known to a person skilled in the art, or be used as salts in subsequent biological assays. It is to be understood that the specific form of a compound of the present invention as isolated and as described herein is not necessarily the only form in which said compound can be applied to a biological assay in order to quantify the specific biological activity.

All the starting materials and reagents are commercially available and were used as is. ¹H Nuclear magnetic resonance (NMR) spectroscopy was carried out using a Bruker instrument operating at 400 MHz using the stated solvent at around room temperature unless otherwise stated. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; dt, doublet of triplets; m, multiplet; br, broad.

Chemical names were generated using the ChemDraw naming software (Version 17.0.0.206) by PerkinElmer Informatics, Inc. In some cases, generally accepted names and generally accepted acronyms for commercially available reagents were used in place of names generated by the naming software.

### Intermediate A1

### Synthesis of 2,3-dihydro-1H-inden-4-ol (A1)

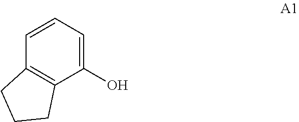

A mixture of 4-hydroxy-2,3-dihydro-1H-inden-1-one (500 mg, 3.37 mmol, 1.0 eq), Zn (883 mg, 13.5 mmol, 4.0 eq) and con. HCl (3 mL) in 1,4-dioxane (8 mL) was stirred at 100° C. overnight. The mixture was filtered, concentrated to dryness, and the residue purified by RP column to afford Intermediate A1 (300 mg, 66% yield) as a yellow solid.

TLC: EtOAc/pet. ether=1/5 (v/v), Rf=0.70

¹H NMR: (400 MHz, DMSO-d₆) δ 9.10 (s, 1H), 6.94-6.88 (m, 1H), 6.65 (dd, J=7.3, 1.0 Hz, 1H), 6.54 (dd, J=7.9, 0.9 Hz, 1H), 2.80 (t, J=7.5 Hz, 2H), 2.73 (t, J=7.4 Hz, 2H), 1.95 (q, J=7.4 Hz, 2H).

### Intermediate A2

### Synthesis of 3-methyl-1H-inden-7-ol (A2)

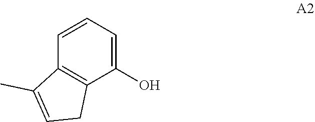

To a solution of 4-hydroxy-2,3-dihydro-1H-inden-1-one (2.0 g, 13.5 mmol, 1.0 eq) in THF (20 mL) at 0° C. was added dropwise MeMgBr (2 M in THF, 20 mL, 40.0 mmol, 3 eq). The mixture was stirred at rt for 2 h, quenched with water (80 mL), and extracted with EtOAc (20 mL). The organic layer was washed with brine (50 mL) and concentrated to provide a residue that was dissolved in DCM (20 mL), and treated with HCl (2 M, 18.0 mL). The biphasic mixture was stirred vigorously at rt for 2 h, then the organic layer was separated, washed with water (20 mL) and brine (20 mL), and concentrated in vacuo. The residue was purified by silica gel column chromatography (pet. ether/EtOAc=10/1 to 2/1) to afford Intermediate A2 (550 mg, 310% yield) as a yellow solid.

¹H NMR: (400 MHz, DMSO-d₆) δ 9.28 (s, 1H), 7.11 (t, J=7.7 Hz, 1H), 6.82 (dd, J=7.4, 0.8 Hz, 1H), 6.64 (d, J=7.9 Hz, 1H), 6.19 (q, J=1.8 Hz, 1H), 3.17 (p, J=2.2 Hz, 2H), 2.08 (q, J=2.0 Hz, 3H).

### Intermediate A3

### Synthesis of 1-methyl-2,3-dihydro-1H-inden-4-ol (A3)

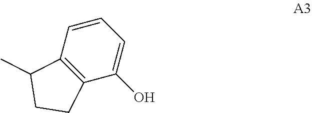

A mixture of A2 (500 mg, 3.42 mmol) and Pd/C (100 mg) in MeOH (10 mL) was degassed under reduced pressure and refilled with H₂ three times. The reaction mixture was stirred under 1 atm H₂ at rt for 2 h. The mixture was filtered, and concentrated to dryness to afford Intermediate A3 (460 mg, 90.8% yield) as a yellow oil.

¹H NMR: (400 MHz, DMSO-d₆) δ 9.10 (s, 1H), 6.95 (t, J=7.7 Hz, 1H), 6.63 (d, J=7.4 Hz, 1H), 6.56 (d, J=7.9 Hz, 1H), 3.07 (p, J=7.2 Hz, 1H), 2.78 (ddd, J=15.8, 8.7, 3.8 Hz, 1H), 2.60 (dt, J=16.1, 8.2 Hz, 1H), 2.22 (dtd, J=11.9, 8.0, 3.8 Hz, 1H), 1.48 (dq, J=12.3, 8.4 Hz, 1H), 1.19 (d, J=6.9 Hz, 3H).

### Intermediate A4

### Synthesis of 6-methyl-2,3-dihydro-1H-inden-4-ol (A4)

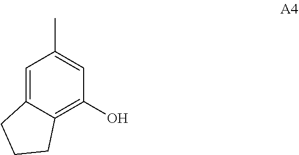

A solution of 7-hydroxy-5-methyl-2,3-dihydro-1H-inden-1-one (2.5 g, 15.41 mmol, 1.0 eq), Zn (5.04 g, 77.1 mmol, 5.0 eq) and con. HCl (30 mL) in toluene (30 mL) was stirred at 100° C. for 48 h. The mixture was concentrated to dryness, H₂O (30 mL) was added, and the mixture was extracted with EtOAc (25 mL*2). The combined organic layer was washed with brine (50 mL), dried over Na₂SO₄, and concentrated in vacuo. The residue was purified with RP column chromatography to afford Intermediate A4 (1.5 g, 66% yield) as a yellow solid.

TLC: EtOAc/pet. ether=1/5 (v/v), Rf=0.70

¹H NMR: (400 MHz, DMSO-d₆) δ 8.97 (s, 1H), 6.47 (s, 1H), 6.36 (s, 1H), 2.75 (t, J=7.5 Hz, 2H), 2.67 (t, J=7.3 Hz, 2H), 2.16 (s, 3H), 1.93 (q, J=7.4 Hz, 2H).

### Intermediate A5

### Synthesis of ethyl 2-((4-formylnaphthalen-1-yl)oxy)acetate (A5)

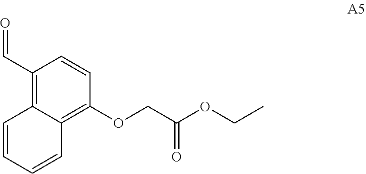 [SMILES: CCOC(=O)COc1ccc(C=O)c2ccccc12]

To a solution of 4-hydroxy-1-naphthaldehyde (0.80 g, 4.8 mmol, 1.0 eq) and ethyl bromoacetate (1.0 g, 5.8 mmol, 1.2 eq) in DMF (10 mL) at rt was added Cs₂CO₃ (2.4 g, 7.23 mmol, 1.5 eq) under N₂ (g). The reaction was stirred overnight, then diluted with EtOAc (20 mL) and filtered. The filtrate was washed with water (50 mL*2) and brine (50 mL*2), dried over Na₂SO₄, and concentrated in vacuo. The crude product was purified silica gel flash column chromatography (EtOAc/pet. ether=1/15) to afford Intermediate A5 (700 mg, 56% yield) as a yellow solid.

TLC: EtOAc/pet. ether-1/3 (v/v), Rf=0.45

¹H NMR: (400 MHz, DMSO-d6) δ 10.20 (s, 1H), 9.22 (ddd, J=8.4, 1.2, 0.4 Hz, 1H), 8.34 (ddd, J=8.8, 1.6, 0.8 Hz, 1H), 8.14 (d, J=8.0 Hz, 1H), 7.77 (ddd, J=8.4, 6.8, 1.2 Hz, 1H), 7.68 (ddd, J=8.0, 6.8, 1.2 Hz, 1H), 7.17 (d, J=8.0 Hz, 1H), 5.17 (s, 2H), 4.22 (q, J=7.2 Hz, 2H), 1.25 (d, J=6.8 Hz, 3H).

### Intermediate A6

### Synthesis of 5-fluoro-2,3-dihydro-1H-inden-4-ol (A6)

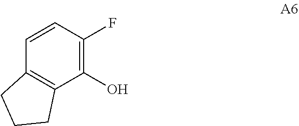 [SMILES: Oc1c(F)ccc2c1CCC2]

A solution of 2,3-dihydro-1H-inden-4-ol (1.0 g, 7.4 mmol, 1.0 eq) and N-fluorodibenzenesulfonamide (NFSI, 2.6 g, 8.2 mmol, 1.1 eq) in a sealed tube was heated to 90° C. under microwave irradiation for 2 h. The reaction mixture was diluted with EtOAc (50 mL), washed with water (20 mL*2), dried over Na₂SO₄, and concentrated in vacuo. The crude product was purified through silica gel flash column chromatography (EtOAc/pet. ether=1/10) to afford Intermediate A6 (125 mg, 11.0% yield) as a light-yellow oil.

¹H NMR: (400 MHz, DMSO-d₆) δ 9.27 (s, 1H), 6.86 (dd, J=11.5, 8.0 Hz, 1H), 6.62 (dd, J=8.1, 4.2 Hz, 1H), 2.78 (t, J=7.5 Hz, 4H), 2.05-1.95 (m, 2H).

### Intermediate A7

### Synthesis of 5-fluoro-7-iodo-2,3-dihydro-1H-inden-4-ol (A7)

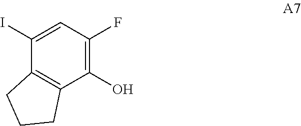 [SMILES: Oc1c(F)cc(I)c2c1CCC2]

To a solution of compound A6 (500 mg, 3.3 mmol, 1.0 eq) in DCM (10 mL) at rt was added NIS (740 mg, 3.3 mmol, 1.0 eq) and pyridinium 4-toluenesulfonate (170 mg, 0.70 mmol, 0.2 eq) with stirring overnight. The reaction mixture was diluted with DCM (50 mL), washed with water (10 mL*2) and brine (10 mL*2), dried over Na₂SO₄, and concentrated in vacuo. The crude product was purified through Prep-TLC (EtOAc/pet. ether=1/10) to afford Intermediate A7 (272 mg, 29.7% yield) as a light-yellow oil.

¹H NMR: (400 MHz, DMSO-d₆) δ 9.62 (s, 1H), 7.31 (d, J=10.4 Hz, 1H), 2.95 (t, J=7.6 Hz, 2H), 2.73 (t, J=7.4 Hz, 2H), 2.05-1.98 (m, 2H).

### Intermediate A8

### Synthesis of ((5-fluoro-7-iodo-2,3-dihydro-1H-inden-4-yl)oxy)triisopropylsilane (A8)

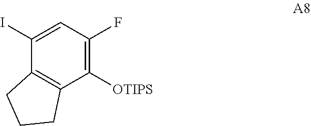

To a solution of compound A7 (270 mg, 1.0 mmol, 1.0 eq) in DCM (10 mL) at rt was added TIPSCl (243 mg, 1.3 mmol, 1.3 eq), TEA (200 mg, 2.0 mmol, 2.0 eq) and DMAP (12 mg, 1.2 mmol, 1.2 eq), and the reaction mixture was stirred overnight. The reaction mixture was diluted with DCM (50 mL), washed with brine (20 mL*2), dried over Na₂SO₄, and concentrated in vacuo. The crude product was purified through Prep-TLC (EtOAc/pet. ether=1/200) to afford Intermediate A8 (220 mg, 52.1% yield) as a colorless oil.

¹H NMR: (400 MHz, DMSO-d₆) δ 7.41 (d, J=10.5 Hz, 1H), 3.00 (t, J=7.5 Hz, 2H), 2.76 (t, J=7.5 Hz, 2H), 2.11-1.93 (m, 2H), 1.23 (t, J=7.5 Hz, 4H), 1.04 (d, J=7.4 Hz, 18H).

### Intermediate A9

### Synthesis of 6-fluoro-7-((triisopropylsilyl)oxy)-2,3-dihydro-1H-indene-4-carbaldehyde (A9)

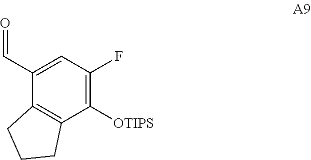

To a solution of compound A8 (220 mg, 0.50 mmol, 1.0 eq) in THF (5 mL) at −60° C. was added n-BuLi (2.5 M, 400 μL, 1.0 mmol, 2.0 eq), dropwise. After 15 min, DMF (74 mg, 1.0 mmol, 2.0 eq) was added at −60° C. The reaction was quenched with water (20 mL), acidified to pH=3 with aqueous HCl (1N), and extracted with EtOAc (20 mL*2). The combined organic layer was dried over Na₂SO₄ and concentrated in vacuo to afford Intermediate A9 (160 mg, 93.8% yield) as a light yellow oil.

¹H NMR: (400 MHz, DMSO-d₆) δ 9.93 (d, J=1.4 Hz, 1H), 7.54 (d, J=11.4 Hz, 1H), 3.19 (t, J=7.6 Hz, 2H), 2.86 (t, J=7.5 Hz, 2H), 2.16-2.02 (m, 2H), 1.34-1.24 (m, 3H), 1.06 (d, J=7.5 Hz, 18H).

### Intermediate A10

### Synthesis of 6-fluoro-7-hydroxy-2,3-dihydro-1H-indene-4-carbaldehyde (A10)

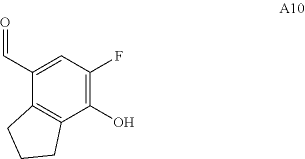 [SMILES: O=Cc1cc(F)c(O)c2c1CCC2]

To a solution of compound A9 (160 mg, 0.47 mmol, 1.0 eq) in THF (5 mL) at rt was added, dropwise, TBAF (1 Min THF, 620 μL, 0.62 mmol, 1.3 eq). After 3 h the reaction mixture was diluted with EtOAc (30 mL), washed with water (10 mL*2) and brine (10 mL*2), dried over Na₂SO₄, and concentrated in vacuo to afford Intermediate A10 (80 mg, 93.3% yield) as a light yellow oil.

### Intermediate A11

### Synthesis of ethyl 2-((5-fluoro-7-formyl-2,3-dihydro-1H-inden-4-yl)oxy)acetate (A11)

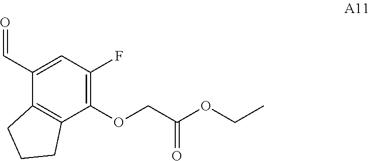 [SMILES: CCOC(=O)COc1c(F)cc(C=O)c2c1CCC2]

To a stirred solution of Intermediate A10 (80 mg, 0.44 mmol, 1.0 eq) and ethyl bromoacetate (89 mg, 0.53 mmol, 1.2 eq) in DMF (5 mL) at rt was added K₂CO₃ (92 mg, 0.66 mmol, 1.5 eq). After 3 h the reaction mixture was diluted with water (30 mL), and extracted with EtOAc (20 mL*2). The combined organic phase was washed with brine (10 mL*2), dried over Na₂SO₄, and concentrated in vacuo. The crude product was purified through Prep-TLC (EtOAc/pet. ether=1/10) to afford Intermediate A11 (55 mg, 46.5% yield) as a colorless oil.

¹H NMR: (400 MHz, DMSO-d₆) δ 9.96 (s, 1H), 7.58-7.49 (m, 1H), 4.96 (d, J=2.7 Hz, 2H), 4.16 (qd, J=7.3, 2.5 Hz, 2H), 3.18 (t, J=7.9 Hz, 2H), 2.91 (t, J=7.4 Hz, 2H), 2.15-2.04 (m, 2H), 1.19 (dt, J=7.2, 3.9 Hz, 3H).

### Intermediate B1

### Synthesis of 3-fluoro-2-(prop-1-en-2-yl)phenol (B1)

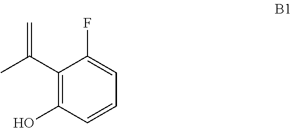

To a mixture of 2-bromo-3-fluorophenol (38.0 g, 200 mmol, 1 eq), isopropenyl-2-boron(pinacolate) (50.4 g, 300 mmol, 1.5 eq) and Pd(dppf)Cl₂·CH₂Cl₂ (16 g, 20 mmol, 0.1 eq) in 1,4-dioxane (300 mL) and water (30 mL) at rt was added K₂CO₃ (55.3 g, 400 mmol, 2.0 eq). The mixture was heated to 70° C. and stirred overnight. The reaction mixture was cooled to rt, quenched with water (100 mL) and extracted with EtOAc (100 mL*3). The combined organic phase was washed with brine (200 mL), dried over Na₂SO₄, concentrated in vacuo and purified by silica gel column chromatography (EtOAc/pet. ether=1/100 to 1/20) to afford Intermediate B1 (23 g, 76% yield) as a white solid.

TLC: EtOAc/pet. ether=1/10 (v/v), Rf=0.55

¹H NMR: (400 MHz, DMSO-d₆) δ 9.72 (s, 1H), 7.06 (td, J=8.4, 6.8 Hz, 1H), 6.66 (td, J=8.4, 1.2 Hz, 1H), 6.59 (m, 1.0 Hz, 1H), 5.28 (m, 1H), 4.89 (m, 1H), 1.98 (s, 3H).

### Intermediate B2

### Synthesis of 3-fluoro-2-isopropylphenol (B2)

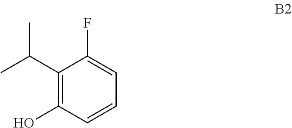

To a solution of Intermediate B1 (23.0 g, 151 mmol) in MeOH (300 mL) was added Pd/C (10%) (6.0 g). The reaction mixture was stirred at 60° C. overnight under an atmosphere of hydrogen (1 atm). The mixture was cooled to rt, filtered, and concentrated in vacuo to afford Intermediate B2 (21.0 g, 90% yield) as a yellow oil.

TLC: EtOAc/pet. ether=1/50 (v/v), R_{f}=0.25

¹H NMR: (400 MHz, DMSO-d₆) δ 9.69 (s, 1H), 7.00-6.93 (m, 1H), 6.65-6.60 (m, 1H), 6.52 (ddd, J=10.8, 8.0, 1.2 Hz, 1H), 3.40 (m, 1H), 1.25 (dd, J=7.2, 1.2 Hz, 6H).

### Intermediate B3

### Synthesis of 2-(1-(4-fluorophenyl)-1-hydroxyethyl)phenol (B3)

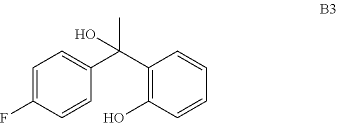 [SMILES: CC(O)(c1ccc(F)cc1)c1ccccc1O]

A solution of 2-bromophenol (20.0 g, 116 mmol, 1.0 eq) in THF (100 mL) was cooled to −78° C. n-BuLi (2.5 M, 92.5 mL, 232 mmol, 2.0 eq) was added dropwise, the mixture was stirred at rt for 1 h, and was then cooled back to −78° C. 4-fluoroacetophenone (16.0 g, 116 mmol, 1.0 eq) in THF (10 mL) was added, and the mixture was stirred at rt for 16 h. The reaction mixture was acidified to pH˜6-7 with 2N HCl and was then extracted with EtOAc (50 mL*3). The combined organic phase was washed with brine (100 mL), dried over Na₂SO₄, and concentrated in vacuo. The crude product was purified by reversed-phase column chromatography to afford Intermediate B3 (2.0 g, 7.30% yield).

TLC: EtOAc/pet. ether=1/5 (v/v), Rf=0.3

LCMS: RT=3.46 min; [M−1]=231.1.

### Intermediate B4

### Synthesis of 2-(1-(4-fluorophenyl)ethyl)phenol (B4)

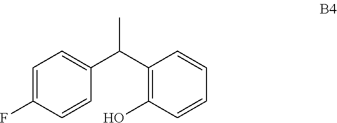 [SMILES: CC(c1ccc(F)cc1)c1ccccc1O]

To a solution of Intermediate B3 (5.7 g, 25 mmol, 1.0 eq) in DCM (50 mL) at 0° C. were added Et₃SiH (11.4 g, 98.0 mmol, 4.0 eq) and TFA (84.0 g, 735 mmol, 30 eq). The mixture was stirred at rt for 2 h. The reaction was concentrated in vacuo and purified by silica gel column chromatography (pet. ether/EtOAc=10/1) to afford Intermediate B4 (5.0 g, 94% yield).

TLC: EtOAc/pet. ether=1/5 (v/v), Rf=0.25

¹H NMR: (400 MHz, DMSO) δ 9.33 (s, 1H), 7.28-7.19 (m, 2H), 7.12-7.02 (m, 3H), 6.99 (m, 1H), 6.79-6.71 (m, 2H), 4.44 (d, J=7.3 Hz, 1H), 1.49 (d, J=7.3 Hz, 3H).

### Intermediate B5

### Synthesis of 4-iodo-2-isopropylphenol (B5)

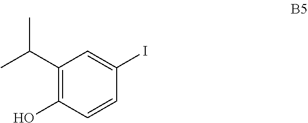

To a solution of 2-isopropylphenol (840 g, 6.17 mol, 1.0 eq) in methanol (10 L) were added NaI (925 g, 6.17 mol, 1.0 eq) and NaOH (246.7 g, 6.17 mol, 1.0 eq). The mixture was cooled to −10° C. and sodium hypochlorite (9.6 L, 6.17 mol, 15% in water) was added dropwise over 4 h. The mixture was quenched by slowly adding 10% aq. Na₂S₂O₃ solution (5 L) with stirring; the mixture was acidified with concentrated aqueous HCl. The mixture was extracted with EtOAc (5 L*2). The combined organic phase was washed with brine (5 L), dried over Na₂SO₄, and concentrated in vacuo. The residue was purified by silica gel column chromatography (pet. ether/EtOAc=100/1 to 20/1) to afford Intermediate B5 (800 g, 49% yield) as a reddish oil.

¹H NMR: (400 MHz, DMSO-d₆) δ 10.08 (s, 1H), 7.85 (d, J=2.3 Hz, 1H), 7.80 (dd, J=8.4, 2.3 Hz, 1H), 7.13 (d, J=8.4 Hz, 1H), 3.64 (m, 1H), 1.64 (d, J=6.9 Hz, 6H).

### Intermediate B6

### Synthesis of 4-iodo-2-isopropyl-1-(methoxymethoxy)benzene (B6)

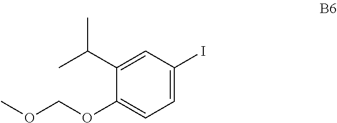

To a solution of Intermediate B5 (283 g, 1.08 mol, 1.0 eq) in DMF (3.28 L) were added MOM-Cl (258 g, 3.24 mol, 3.0 eq) and Cs₂CO₃ (1.05 kg, 3.24 mol, 3.0 eq). The mixture was stirred at rt for 3 h under N₂ atmosphere. The mixture was diluted with water (10 L) and extracted with EtOAc (5 L×2). The combined organic layers were dried over Na₂SO₄ and concentrated in vacuo. The residue was purified by silica gel column chromatography (pet. ether/EtOAc=100/1 to 30/1) to afford Intermediate B6 (250 g, 76% yield) as a reddish oil.

¹H NMR: (400 MHz, DMSO-d₆) δ7.45 (d, J=8.0 Hz, 2H), 6.87 (d, J=8.0 Hz, 1H), 5.20 (s, 2H), 3.37 (s, 3H), 3.26-3.18 (m, 1H), 1.14 (d, J=8.0 Hz, 6H).

### Intermediate C1

### Synthesis of 7-(hydroxymethyl)-2,3-dihydro-1H-inden-4-ol (C1)

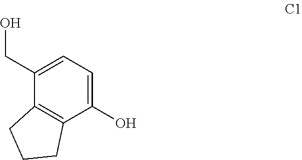 [SMILES: OCc1ccc(O)c2c1CCC2]

To a solution of A1 (350 mg, 2.61 mmol, 1.0 eq) and NaOH (115 mg, 2.87 mmol, 1.1 eq) in water (5 mL) at 50° C. was added dropwise formaldehyde (40%, 78 mg, 2.6 mmol, 1.1 eq). The mixture was stirred at 50° C. for 2 h then acidified to pH=5-6 with 2N HCl and extracted with EtOAc (10 mL). The organic layer was concentrated, washed with brine (10 mL), and purified by Prep-TLC (EtOAc/pet. ether=1/1) to afford Intermediate C1 (100 mg, 23.4% yield) as a white solid.

TLC: EtOAc/pet. ether=1/1 (v/v), Rf=0.67

¹H NMR: (400 MHz, DMSO-d₆) δ 9.00 (s, 1H), 6.91 (d, J=8.0 Hz, 1H), 6.52 (d, J=8.0 Hz, 1H), 4.76 (t, J=5.5 Hz, 1H), 4.33 (d, J=5.4 Hz, 2H), 2.79 (t, J=7.5 Hz, 2H), 2.72 (t, J=7.4 Hz, 2H), 2.02-1.91 (m, 2H).

### Intermediate C2

### Synthesis of 7-(hydroxymethyl)-1-methyl-2,3-dihydro-1H-inden-4-ol (C2)

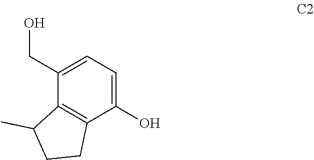

To a solution of A3 (400 mg, 2.70 mmol, 1.0 eq) and NaOH (108 mg, 2.70 mmol, 1.0 eq) in water (10 mL) at 45° C. was added formaldehyde (40%, 81 mg, 2.70 mmol, 1.0 eq) dropwise and the mixture was stirred for 2 h. The mixture was acidified to pH=5-6 with 1 N HCl, then extracted with EtOAc (10 mL). The organic layer was washed with brine (20 mL) and concentrated to dryness. The residue was purified by Prep-TLC to afford Intermediate C2 (50 mg, 10% yield) as a yellow oil.

¹H NMR: (400 MHz, DMSO-d₆) δ 9.00 (s, 1H), 6.94 (d, J=8.1 Hz, 1H), 6.53 (d, J=8.1 Hz, 1H), 4.80 (t, J=5.4 Hz, 1H), 4.47-4.33 (m, 2H), 3.32-3.26 (m, 1H), 2.81-2.62 (m, 2H), 2.14-2.03 (m, 1H), 1.70-1.63 (m, 1H), 1.09 (d, J=7.0 Hz, 3H).

### Intermediate C3

### Synthesis of 7-(hydroxymethyl)-6-methyl-2,3-dihydro-1H-inden-4-ol (C3)

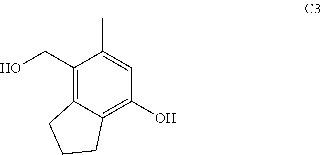

To a solution of A4 (1.0 g, 6.8 mmol, 1.0 eq) and NaOH (297 mg, 7.42 mmol, 1.1 eq) in water (10 mL) at 50° C. was added dropwise formaldehyde (40%, 557 mg, 7.42 mmol, 1.1 eq), and the mixture was stirred at 50° C. for 2 h. The mixture was acidified to pH=5-6 with 2N HCl and was extracted with EtOAc (10 mL). The organic layer was concentrated, washed with brine (10 mL), purified by Prep-TLC (EtOAc/pet. ether=1/1) to afford Intermediate C3 (600 mg, 50% yield) as a yellow solid.

TLC: EtOAc/pet. ether=1/1 (v/v), Rf=0.67

¹H NMR: (400 MHz, DMSO-d₆) δ 8.94 (s, 1H), 6.38 (s, 1H), 4.47 (t, J=5.2 Hz, 1H), 4.33 (d, J=5.3 Hz, 2H), 2.85 (t, J=7.5 Hz, 2H), 2.69 (t, J=7.3 Hz, 2H), 1.97-1.92 (m, 2H).

### Intermediate D1

### Synthesis of ethyl 2-((7-(hydroxymethyl)-2,3-dihydro-1H-inden-4-yl)oxy)acetate (D1)

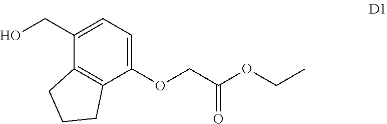 [SMILES: CCOC(=O)COc1ccc(CO)c2c1CCC2]

A solution of C1 (100 mg, 0.61 mmol, 1.0 eq), ethyl bromoacetate (150 mg, 0.91 mmol, 1.5 eq) and K₂CO₃ (253 mg, 1.83 mmol, 3.0 eq) in DMF (3 mL) was stirred at rt for 1 h. H₂O (30 mL) was added and the mixture was extracted with EtOAc (15 mL*2). The combined organic layer was washed with water (20 mL*3) and brine (20 mL), dried over Na₂SO₄, and concentrated to dryness to afford Intermediate D1 (120 mg, 78.7% yield) as a white solid.

TLC: EtOAc/pet. ether=1/5 (v/v), R_{f}=0.41

¹H NMR: (400 MHz, DMSO-d₆) δ 7.05 (d, J=8.2 Hz, 1H), 6.60 (d, J=8.2 Hz, 1H), 4.74 (s, 2H), 4.38 (s, 2H), 4.15 (q, J=7.1 Hz, 2H), 2.82 (q, J=7.8 Hz, 4H), 2.01 (p, J=7.5 Hz, 2H), 1.20 (t, J=7.1 Hz, 3H).

### Intermediate D2

### Synthesis of ethyl 2-((7-(hydroxymethyl)-1-methyl-2,3-dihydro-1H-inden-4-yl)oxy)acetate (D2)

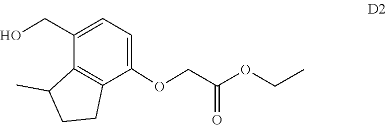 [SMILES: CCOC(=O)COc1ccc(CO)c2c1CCC2C]

A mixture of C2 (40 mg, 220 μmol, 1.0 eq), K₂CO₃ (47 mg, 340 μmol, 1.5 eq) and ethyl bromoacetate (45 mg, 270 μmol, 1.2 eq) in DMF (2 mL) was stirred at rt for 2 h. H₂O (10 mL) was added and the mixture was extracted with EtOAc (5 mL*2). The combined organic layer was washed with H₂O (15 mL*4) and brine (10 mL*2), dried over Na₂SO₄, and concentrated to dryness to afford Intermediate D2 (45 mg, 76% yield) as a white solid.

¹H NMR: (400 MHz, DMSO-d₆) δ 7.08 (d, J=8.3 Hz, 1H), 6.61 (d, J=8.3 Hz, 1H), 4.74 (s, 2H), 4.44 (q, J=12.8 Hz, 2H), 4.18-4.13 (m, 3H), 2.89-2.71 (m, 2H), 2.13 (dq, J=12.5, 9.1 Hz, 1H), 1.76-1.66 (m, 1H), 1.21 (t, J=7.1 Hz, 3H), 1.11 (d, J=7.0 Hz, 3H).

### Intermediate D3

### Synthesis of ethyl 2-((7-(hydroxymethyl)-6-methyl-2,3-dihydro-1H-inden-4-yl)oxy)acetate (D3)

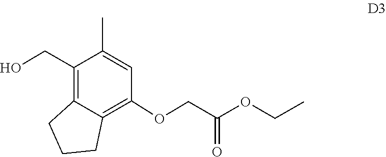 [SMILES: CCOC(=O)COc1cc(C)c(CO)c2c1CCC2]

A solution of C3 (600 mg, 3.37 mmol, 1.0 eq), ethyl bromoacetate (675 mg, 4.04 mmol, 1.2 eq) and K₂CO₃ (698 mg, 5.05 mmol, 1.5 eq) in DMF (10 mL) was stirred at rt for 1 h. H₂O (50 mL) was added, and the mixture was extracted with EtOAc (25 mL*2). The combined organic layer was washed with water (30 mL*3) and brine (50 mL), dried over Na₂SO₄, and concentrated to dryness to afford Intermediate D4 (800 mg, 90% yield) as a white solid.

TLC: EtOAc/pet. ether=1/5 (v/v), R_{f}=0.41

¹H NMR: (400 MHz, DMSO-d₆) δ 6.46 (s, 1H), 4.73 (s, 2H), 4.58 (t, J=5.3 Hz, 1H), 4.37 (d, J=5.4 Hz, 2H), 4.16 (q, J=7.1 Hz, 2H), 2.90 (t, J=7.4 Hz, 2H), 2.77 (t, J=7.4 Hz, 2H), 2.27 (s, 3H), 2.03-1.96 (m, 2H), 1.21 (t, J=7.1 Hz, 3H).

### Intermediate D4

### Synthesis of ethyl 2-((5-fluoro-7-(hydroxymethyl)-2,3-dihydro-1H-inden-4-yl)oxy)acetate (D4)

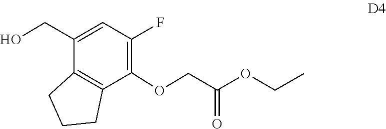 [SMILES: CCOC(=O)COc1c(F)cc(CO)c2c1CCC2]

To a stirred solution of Intermediate A11 (55 mg, 0.21 mmol, 1.0 eq) in THF (5 mL) at rt was added portionwise NaBH₄ (8.0 mg, 0.21 mmol, 1.0 eq). After 5 h the reaction was quenched with saturated aqueous NH₄Cl (20 mL), and extracted with EtOAc (10 mL*2). The combined organic layer was dried over Na₂SO₄ and concentrated in vacuo to afford Intermediate D4 (50 mg, 90% yield) as a light yellow oil.

### Intermediate E1

### Synthesis of ethyl 2-((7-(chloromethyl)-2,3-dihydro-1H-inden-4-yl)oxy)acetate (E1)

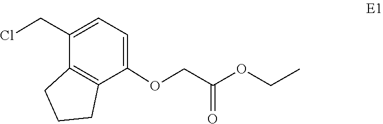 [SMILES: CCOC(=O)COc1ccc(CCl)c2c1CCC2]

A solution of D1 (110 mg, 0.44 mmol, 1.0 eq) and SOCl₂ (78 mg, 0.66 mmol, 1.5 eq) in DCM (5 mL) was stirred at 0° C. for 2 h. The mixture was concentrated to dryness to afford E1 (110 mg, 93% yield) as a yellow solid.

TLC: EtOAc/pet. ether=1/5 (v/v), Rf=0.57

¹H NMR: (400 MHz, DMSO-d₆) δ 7.14 (d, J=8.3 Hz, 1H), 6.64 (d, J=8.3 Hz, 1H), 4.79 (s, 2H), 4.70 (s, 2H), 4.16 (q, J=7.1 Hz, 2H), 2.94 (t, J=7.5 Hz, 2H), 2.83 (t, J=7.5 Hz, 2H), 2.05 (p, J=7.6 Hz, 2H), 1.21 (t, J=7.1 Hz, 3H).

### Intermediate E2

### Synthesis of ethyl 2-((7-(chloromethyl)-1-methyl-2,3-dihydro-1H-inden-4-yl)oxy)acetate (E2)

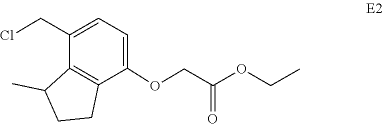 [SMILES: CCOC(=O)COc1ccc(CCl)c2c1CCC2C]

A solution of D2 (40 mg, 150 μmol, 1.0 eq) and SOCl₂ (27 mg, 230 μmol, 1.5 eq) in DCM (2 mL) was stirred at rt for 2 h. The mixture was concentrated to dryness to afford Intermediate E2 (35 mg, 82% yield) as a yellow solid.

### Intermediate E3

### Synthesis of ethyl 2-((7-(chloromethyl)-6-methyl-2,3-dihydro-1H-inden-4-yl)oxy)acetate (E3)

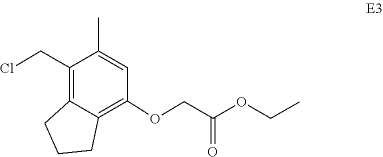 [SMILES: CCOC(=O)COc1cc(C)c(CCl)c2c1CCC2]

A solution of D3 (600 mg, 2.27 mmol, 1.0 eq) and SOC₂ (405 mg, 3.41 mmol, 1.5 eq) in DCM (10 mL) was stirred at 0° C. for 2 h. The mixture was concentrated to dryness to afford Intermediate E3 (600 mg, 94% yield) as a yellow solid.

TLC: EtOAc/pet. ether=1/5 (v/v), Rf=0.57

¹H NMR: (400 MHz, DMSO-d₆) δ 6.55 (s, 1H), 4.78 (s, 2H), 4.71 (s, 2H), 4.19-4.13 (m, 2H), 2.92 (t, J=7.5 Hz, 2H), 2.79 (t, J=7.5 Hz, 2H), 2.31 (s, 3H), 2.03 (p, J=7.3 Hz, 2H), 1.21 (t, J=7.3 Hz, 3H).

### Intermediate E4

### Synthesis of ethyl 2-((7-(chloromethyl)-5-fluoro-2,3-dihydro-1H-inden-4-yl)oxy)acetate (E4)

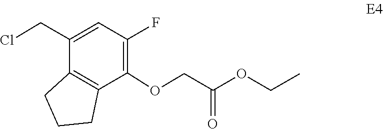 [SMILES: CCOC(=O)COc1c(F)cc(CCl)c2c1CCC2]

To a solution of D4 (50 mg, 0.18 mmol, 1.0 eq) in DCM (5 mL) at rt was added thionyl chloride (67 mg, 0.56 mmol, 3.0 eq). After 6 h the reaction mixture was concentrated under reduce pressure to afford Intermediate E4 (50 mg, 94% yield) as a colorless oil.

### Intermediate F1

### Synthesis of ethyl 2-((4-(hydroxy(3-isopropyl-4-(methoxymethoxy)phenyl)methyl)naphthalen-1-yl)oxy)acetate (F1)

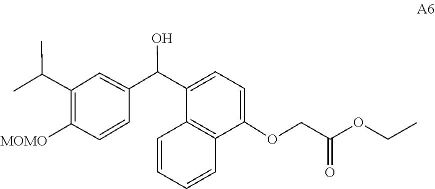

To a solution of B6 (308 mg, 1.01 mmol, 1.3 eq) in THF (3 mL) at −20° C. was added dropwise i-prMgCl (1 M, 2.5 mL, 3.3 eq). The mixture was stirred at rt for 2 h then cooled down to −70° C. and a solution of A5 (200 mg, 0.77 mmol, 1.0 eq) in THF (2 mL) was added. The mixture was stirred at −70° C. for 1 h, then aq. NH₄Cl (2 mL) was added dropwise, and the mixture warmed to rt. The mixture was extracted with EtOAc (15 mL*2) and the combined organic layer was washed with brine (15 mL), dried over Na₂SO₄, and concentrated in vacuo. The crude product was purified through Prep-TLC (pet. ether/EtOAc=3:1) to afford Intermediate F1 (79 mg, 23% yield) as a colorless solid.

TLC: Pet. ether/EtOAc=3/1 (v/v), Rf=0.37

¹H NMR: (400 MHz, DMSO-d₆) δ 8.23 (m, 1H), 8.12 (m, 1H), 7.50-7.44 (m, 3H), 7.33 (d, J=2.0 Hz, 1H), 7.01 (dd, J=8.8, 2.4 Hz, 1H), 6.89 (dd, J=8.4, 4.4 Hz, 2H), 6.23 (d, J=4.5 Hz, 1H), 5.82 (d, J=4.4 Hz, 1H), 5.15 (s, 2H), 4.97 (s, 2H), 4.20 (q, J=7.1 Hz, 2H), 3.37-3.35 (m, 3H), 3.22 (p, J=7.2 Hz, 1H), 1.22 (d, J=7.2 Hz, 3H), 1.13 (d, J=7.2 Hz, 6H).

### Intermediate A1′

### Synthesis of 4-bromo-2-fluoro-5-(trifluoromethyl)phenol (A1′)

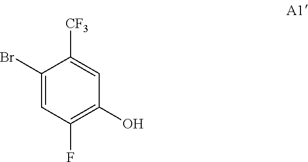 [SMILES: Oc1cc(C(F)(F)F)c(Br)cc1F]

A solution of 4-bromo-2-fluoro-5-(trifluoromethyl)aniline (10.0 g, 38.8 mmol, 1.0 eq) in water (200 mL) and HBF₄ (200 mL, 50% in H₂O) was cooled to 0° C. and NaNO₂ (3.0 g, 43 mmol, 1.1 eq) in water (5 mL) was added dropwise. The reaction was stirred at 0° C. for 30 min, then Cu₂O (5.6 g, 39 mmol, 1.0 eq) and CuSO₄ (50 mL) were added sequentially. The reaction mixture was stirred at rt for 20 min, then extracted with EtOAc (50 mL*2). The combined organic phase was washed with brine, dried over Na₂SO₄ and concentrated in vacuo. The residue was purified by silica gel column chromatography (pet. ether/EtOAc=50/1 to 10/1) to afford the product Intermediate A1′ (2.5 g, 24.9% yield) as a yellow oil.

¹H NMR: (400 MHz, DMSO-d₆) δ 10.89 (s, 1H), 7.80-7.71 (m, 1H), 7.38 (d, J=8.6 Hz, 1H).

### Intermediate A2′

### Synthesis of (4-bromo-2-fluoro-5-(trifluoromethyl)phenoxy)triisopropylsilane (A2′)

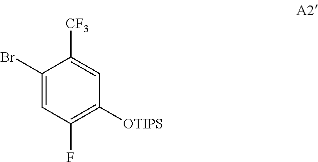

To a mixture of A1′ (1.10 g, 4.25 mmol, 1.0 eq) and triethylamine (0.86 g, 8.5 mmol, 2.0 eq) in DCM (10 mL) was added TIPSOTf (1.56 g, 5.10 mmol, 1.2 eq). The mixture was stirred at rt for 1 h. Water (30 mL) was added and the mixture was extracted with DCM (20 mL*2). The combined organic phase was washed with brine (30 mL), dried over Na₂SO₄ and concentrated in vacuo to afford Intermediate A2′ (1.5 g, 85% yield) as a colorless oil.

¹H NMR: (400 MHz, DMSO-d₆) δ 7.94 (d, J=10.2 Hz, 1H), 7.33 (d, J=8.4 Hz, 1H), 1.31-1.21 (m, 3H), 1.02 (d, J=37.3 Hz, 18H).

### Intermediate A3′

### Synthesis of 5-fluoro-2-(trifluoromethyl)-4-((triisopropylsilyl)oxy)benzaldehyde (A3′)

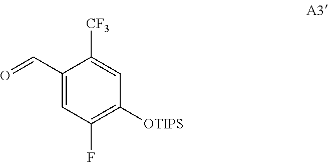

To a mixture of A2′ (1.5 g, 3.6 mmol, 1.0 eq) in THF (20 mL) at −70° C. was added n-BuLi (2.5 M, 1.4 mL, 1.0 eq). The mixture was stirred at −70° C. for 1 h, then DMF (0.32 g, 4.3 mmol, 1.2 eq) was added and the mixture was stirred at −78° C. for 2 h. Water (30 mL) was added and the mixture was extracted with EtOAc (25 mL*2). The combined organic layers were washed with water (30 mL*2), and brine (50 mL), dried over Na₂SO₄ and concentrated to dryness to afford crude Intermediate A3′ (1.0 g, 2.7 mmol, 76% yield) as a brown oil that used directly in next step.

### Intermediate A4′

### Synthesis of 5-fluoro-4-hydroxy-2-(trifluoromethyl)benzaldehyde (A4′)

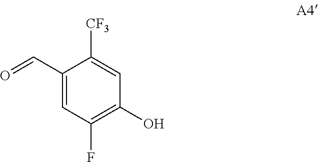 [SMILES: O=Cc1cc(F)c(O)cc1C(F)(F)F]

To a mixture of A3′ (1.0 g, 2.7 mmol, 1.0 eq) in DCM (10 mL) was added TBAF (1 M, 2.74 mL, 1.0 eq). The mixture was stirred at rt for 1 h, then water (30 mL) was added. The organic layer was washed with brine (50 mL), dried over Na₂SO₄ and purified by Prep-TLC (pet. ether/EtOAc=5/1) to afford Intermediate A4′ (200 mg, 35.0% yield) as a colorless oil.

LCMS: T=0.922 min, [M−1]=207.0.

### Intermediate A5′

### Synthesis of ethyl 2-(2-fluoro-4-formyl-5-(trifluoromethyl)phenoxy)acetate (A5′)

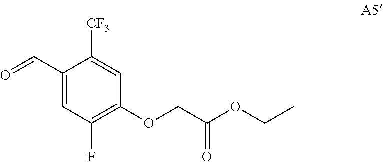 [SMILES: CCOC(=O)COc1cc(C(F)(F)F)c(C=O)cc1F]

A mixture of A4′ (200 mg, 0.96 mmol, 1.0 eq), K₂CO₃ (199 mg, 1.44 mmol, 1.5 eq) and ethyl bromoacetate (193 mg, 1.15 mmol, 1.2 eq) in DMF (2 mL) was stirred at rt for 2 h. Water (20 mL) was added and the mixture was extracted with EtOAc (10 mL*2). The combined organic layers were washed with H₂O (15 mL*4), then brine (10 mL*2), dried over Na₂SO₄ and concentrated to dryness to afford Intermediate A5′ (250 mg, 88.4% yield) as a colorless oil.

¹H NMR: (400 MHz, DMSO-d₆) δ 10.22 (s, 1H), 7.85 (dd, J=7.4, 2.2 Hz, 1H), 7.74-7.70 (m, 1H), 5.13 (s, 2H), 4.19 (q, J=7.1 Hz, 2H), 1.21 (t, J=7.1 Hz, 3H).

### Intermediate A6′

### Synthesis of ethyl 2-(3-chloro-4-methylphenoxy)acetate (A6′)

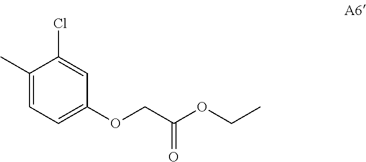

To a solution of 3-chloro-4-methylphenol (5.0 g, 35 mmol, 1.0 eq) in DMF (60 mL) were added K₂CO₃ (7.3 g, 52.60 mmol, 1.5 eq) and ethyl 2-bromoacetate (5.9 g, 35.07 mmol, 1.0 eq). The mixture was stirred at rt for 1 h, then water (300 mL) was added and the mixture was extracted with EtOAc (150 mL). The organic layer was washed with brine (100 mL), dried over Na₂SO₄, and concentrated in vacuo to afford Intermediate A6′ (7.5 g, 94% yield) as a white solid.

TLC: Pet. ether/EtOAc=3/1 (v/v), Rf=0.44.

¹H NMR: (400 MHz, DMSO-d₆) δ 7.24 (dd, J=8.8, 0.8 Hz, 1H), 7.01 (d, J=2.4 Hz, 1H), 6.83 (dd, J=8.4, 2.8 Hz, 1H), 4.78 (s, 2H), 4.16 (q, J=7.2 Hz, 2H), 2.24 (s, 3H), 1.20 (t, J=7.2 Hz, 3H).

### Intermediate A7′

### Synthesis of ethyl 2-(3-bromo-4-methylphenoxy)acetate (A7′)

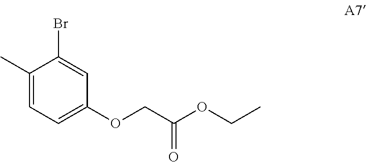

To a solution of 3-bromo-4-methylphenol (5.0 g, 27 mmol, 1.0 eq) in DMF (50 mL) at rt were added K₂CO₃ (7.4 g, 53 mmol, 2.0 eq) and ethyl 2-bromoacetate (5.8 g, 35 mmol, 1.3 eq). The mixture was stirred at rt for 1 h, then water (50 mL) was added and the mixture was extracted with EtOAc (100 mL). The organic phase was washed with brine (100 mL), dried over Na₂SO₄, and concentrated in vacuo to afford Intermediate A7′ (7.3 g, 99% yield) as a colorless oil.

TLC: Pet. ether/EtOAc=3/1 (v/v), Rf=0.89

¹H NMR: (400 MHz, DMSO-d₆) δ 7.25 (d, J=8.4 Hz, 1H), 7.16 (d, J=2.8 Hz, 1H), 6.88 (dd, J=8.4, 2.4 Hz, 1H), 4.78 (s, 2H), 4.19-4.14 (m, 2H), 2.26 (s, 3H), 1.21-1.17 (m, 3H).

### Intermediate A8′

### Synthesis of 3-chloro-2,6-difluoro-4-iodophenol (A8′)

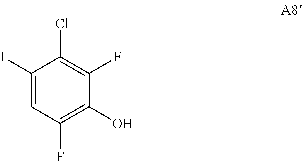 [SMILES: Oc1c(F)cc(I)c(Cl)c1F]

To a solution of 3-chloro-2,6-difluorophenol (4.0 g, 24 mmol, 1.0 eq) in DCM (20 mL) at rt were added N-iodosuccinimide (5.5 g, 24 mmol, 1.0 eq) and p-Toluenesulfonic acid (694 mg, 3.65 mmol, 0.15 eq). The mixture was stirred for 16 hrs at rt; then water (20 mL) was added and the mixture was extracted with DCM (30 mL*3). The combined organic phase was washed with brine (30 mL), dried over Na₂SO₄, concentrated in vacuo and purified by silica gel column chromatography (pet. ether/EtOAc=1/100 to 1/10) to afford Intermediate A8′ (6.2 g, 88% yield) as a yellow solid.

¹H NMR: (400 MHz, DMSO-d₆) δ 11.04 (s, 1H), 7.69 (dd, J=10.1, 2.2 Hz, 1H).

### Intermediate A9′

### Synthesis of (3-chloro-2,6-difluoro-4-iodophenoxy)triisopropylsilane (A9′)

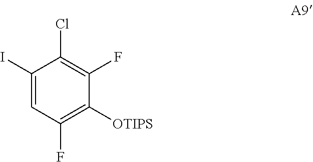

To a mixture of A8′ (3.5 g, 12 mmol, 1.0 eq) and triethylamine (2.4 g, 24 mmol, 2.0 eq) in DCM (30 mL) was added TIPSOTf (4.4 g, 14 mmol, 1.2 eq) and the mixture was stirred at rt for 1 h. Water (30 mL) was added and the mixture was extracted with DCM (20 mL*3).

The combined organic phase was washed with brine (30 mL), dried over Na₂SO₄ and concentrated in vacuo to afford Intermediate A9′ (5.0 g, 93% yield) as a brown oil.

### Intermediate A10

### Synthesis of 2-chloro-3,5-difluoro-4-((triisopropylsilyl)oxy)benzaldehyde (A10″)

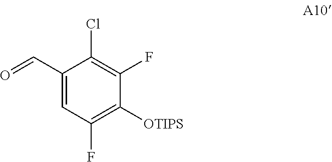

To a solution of A9′ (3.5 g, 7.8 mmol, 1.0 eq) in THF (30 mL) was added iPr-MgCl·LiCl (1.3 M in THF, 6.6 mL, 8.6 mmol, 1.1 eq). The mixture was stirred at rt for 2 h and then DMF (1.15 g, 15.7 mmol, 2.0 eq) was added and the mixture was stirred for 1 h. The mixture was quenched with water (100 mL), and extracted with EtOAc (100 mL*2). The combined organic layers were washed with water (100 mL*3) and brine (200 mL), dried over Na₂SO₄ and concentrated in vacuo to afford Intermediate A10′ (2.5 g, 910% yield), which was used directly in next step.

### Intermediate A11′

### Synthesis of 2-chloro-3,5-difluoro-4-hydroxybenzaldehyde (A11′)

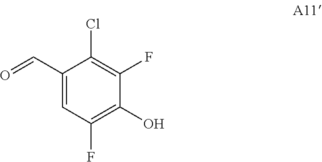 [SMILES: O=Cc1cc(F)c(O)c(F)c1Cl]

To a solution of A10′ (3.5 g, 10 mmol, 1.0 eq) in DCM (30 mL) was added TBAF (1 M in THF, 11 mL, 11 mmol, 1.1 eq) and the mixture was stirred at rt for 20 min. Then, H₂O (50 mL) was added and the mixture was extracted with DCM (25 mL*2). The combined organic layers were washed with water (50 mL) and brine (50 mL), dried over Na₂SO₄, and purified by silica gel column chromatography (pet. ether to pet. ether/EtOAc=10/1) to afford Intermediate A11′ (300 mg, 15.5% yield) as a colorless oil.

¹H NMR: (400 MHz, DMSO-d₆) δ 12.23 (s, 1H), 10.12 (d, J=2.9 Hz, 1H), 7.56 (dd, J=10.7, 2.0 Hz, 1H).

### Intermediate A12′

### Synthesis of ethyl 2-(3-chloro-2,6-difluoro-4-formylphenoxy)acetate (A12′)

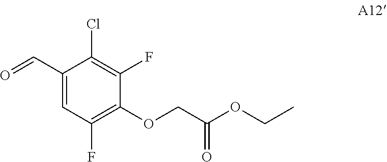 [SMILES: CCOC(=O)COc1c(F)cc(C=O)c(Cl)c1F]

A mixture of A11′ (300 mg, 1.56 mmol, 1.0 eq), K₂CO₃ (312 mg, 1.87 mmol, 1.5 eq) and ethyl bromoacetate (312 mg, 1.87 mmol, 1.2 eq) in DMF (5 mL) was stirred at rt for 2 h. Then, H₂O (20 mL) was added and the mixture was extracted with EtOAc (10 mL*2). The combined organic layer was washed with H₂O (15 mL*4) and brine (10 mL*2), dried over Na₂SO₄, and concentrated to dryness to afford Intermediate A12′ (300 mg, 69.1% yield) as a colorless oil.

¹H NMR: (400 MHz, DMSO-d₆) δ 10.17 (d, J=2.7 Hz, 1H), 7.65 (dd, J=11.5, 2.0 Hz, 1H), 5.11 (d, J=1.5 Hz, 2H), 4.18 (q, J=7.1 Hz, 2H), 1.20 (t, J=7.1 Hz, 3H).

### Intermediate A13′

### Synthesis of 2-(3-bromo-5-chloro-2-fluorophenyl)-4,4,5,5-tetramethyl-1,3,2-dioxaborolane (A13′)

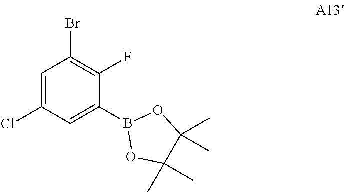 [SMILES: CC1(C)OB(c2cc(Cl)cc(Br)c2F)OC1(C)C]

A mixture of 1-fluoro-2-bromo-4-chlorobenzene (10.0 g, 47.8 mmol), bis(pinacolato)diboron (12.1 g, 47.8 mmol), (1, 5-cyclooctadiene)(methoxy)iridium(I) dimer (633 mg, 955 μmol) and 4,4′-di-tert-butyl-2,2′-bipyridine (320 mg, 1.2 mmol) in THF (100 mL) was stirred at 80° C. overnight under N₂ atmosphere. Water (60 mL) was added and the resultant mixture was extracted with EtOAc (30 mL*3). The combined organic phase was washed with brine (50 mL), dried over Na₂SO₄ and concentrated in vacuo. The crude product was purified by silica gel column chromatography (EtOAc/pet. ether=1/100 to 1/30) to afford Intermediate A13′ (14.0 g, 87.4% yield) as a white solid.

TLC: EtOAc/pet. ether=1/10 (v/v), Rf=0.75

¹H NMR: (400 MHz, DMSO-d₆) δ 7.99 (dd, J=6.0, 2.8 Hz, 1H), 7.54 (dd, J=4.4, 2.8 Hz, 1H), 1.30 (s, 12H).

### Intermediate A14′

### Synthesis of 3-bromo-5-chloro-2-fluorophenol (Intermediate A14′)

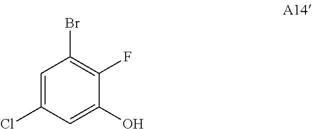

To a solution of Intermediate A13′ (14.0 g, 41.7 mmol) in THF (100 mL) at rt was added H₂O₂ (23.7 mL of 30% aqueous solution, 209 mmol); the mixture was stirred at rt for 2 h. The reaction was quenched with Na₂S₂O₃ (8.0 g) and extracted with EtOAc (50 mL*3). The combined organic phase was washed with brine (100 mL), dried over Na₂SO₄ and concentrated in vacuo to afford Intermediate A14′ (9.0 g, 95.6% yield) as a yellow liquid.

TLC: EtOAc/pet. ether=1/10 (v/v), Rf=0.5

¹H NMR: (400 MHz, DMSO-d₆) δ 10.90 (s, 1H), 7.20 (dd, J=4.8, 2.4 Hz, 1H), 7.00 (dd, J=7.2, 2.4 Hz, 1H).

### Intermediate C1′

### Synthesis of 3-chloro-2-fluoro-4-(hydroxymethyl)phenol (C1′)

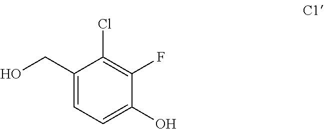 [SMILES: OCc1ccc(O)c(F)c1Cl]

To a solution of NaOH (150 mg, 3.75 mmol, 1.1 eq) in water (5 mL) at rt was added 3-chloro-2-fluorophenol (500 mg, 3.41 mmol, 1.0 eq). The mixture was heated to 45° C., then formaldehyde (37%, 276 mg, 3.41 mmol, 1.0 eq) was added dropwise and the mixture was stirred at 45° C. overnight. After cooling, the reaction was diluted with water (10 mL), acidified with 1 N HCl to pH=6-7 and extracted with EtOAc (5 mL*3). The combined organic phase was washed with brine (10 mL), dried over Na₂SO₄, and concentrated in vacuo. The crude product was purified by silica gel column chromatography (EtOAc/pet. ether=1/3) to afford Intermediate C1′(250 mg, 41.5% yield) as an off-white solid.

TLC: EtOAc/pet. ether=1/3 (v/v), Rf=0.3

¹H NMR: (400 MHz, DMSO-d₆) δ 10.16 (s, 1H), 7.15-7.10 (m, 1H), 6.92 (t, J=8.4 Hz, 1H), 5.24 (t, J=5.6 Hz, 1H), 4.46 (d, J=5.6 Hz, 2H).

### Intermediate C2′

### Synthesis of 5-chloro-2-fluoro-4-(hydroxymethyl)phenol (C2′)

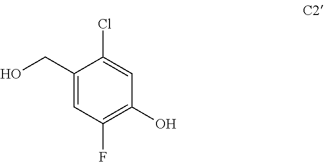 [SMILES: OCc1cc(F)c(O)cc1Cl]

To a solution of NaOH (150 mg, 3.75 mmol, 1.1 eq) in water (5 mL) at rt was added 5-chloro-2-fluorophenol (500 mg, 3.41 mmol, 1.0 eq). The mixture was heated to 45° C., then formaldehyde (37%, 276 mg, 3.41 mmol, 1.0 eq) was added dropwise. The mixture was stirred at 45° C. overnight, then diluted with water (10 mL), acidified with 1 N HCl to pH=6-7 and extracted with EtOAc (5 mL*3). The combined organic layers were washed with brine (10 mL), dried over Na₂SO₄, and concentrated in vacuo. The crude product was purified by silica gel column chromatography (EtOAc/pet. ether=1/3) to afford Intermediate C2′ (440 mg, 73.0% yield) as an off-white solid.

TLC: EtOAc/pet. ether-1/3 (v/v), Rf=0.3

¹H NMR: (400 MHz, DMSO-d₆) δ 10.19 (s, 1H), 7.23 (d, J=12.0 Hz, 1H), 6.96 (d, J=8.0 Hz, 1H), 5.31 (t, J=5.6 Hz, 1H), 4.43 (d, J=4.8 Hz, 2H).

### Intermediate C3′

### Synthesis of 5-bromo-2-fluoro-4-(hydroxymethyl)phenol (C3′)

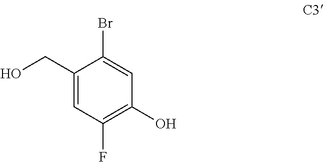 [SMILES: OCc1cc(F)c(O)cc1Br]

To a solution of NaOH (1.0 g, 26 mmol, 1.0 eq) in water (30 mL) at rt was added 4-bromo-2-fluorophenol (5.0 g, 26 mmol, 1.0 eq). The mixture was heated to 45° C., then formaldehyde (37%, 2.1 g, 26 mmol, 1.0 eq) was added dropwise and the mixture was stirred at 45° C. overnight. After cooling, the reaction was diluted with water (20 mL), acidified with 1 N HCl to pH=5-6 and extracted with EtOAc (20 mL*2). The combined organic layers were washed with brine (30 mL), dried over Na₂SO₄, and concentrated in vacuo. The crude product was purified by silica gel column chromatography (pet. ether/EtOAc=30/1 to 10/1) to afford Intermediate C3′ (2.8 g, 48% yield) as a white solid.

TLC: Pet. ether/EtOAc=3/1 (v/v), Rf=0.43.

LCMS: T=0.498 min, [M−1]=218.9.

¹H NMR: (400 MHz, DMSO-d₆) δ 10.19 (s, 1H), 7.22 (d, J=12.0 Hz, 1H), 7.12 (d, J=8.0 Hz, 1H), 5.36 (t, J=5.6 Hz, 1H), 4.38 (d, J=5.6 Hz, 2H).

### Intermediate C4′

### Synthesis of 3-bromo-5-chloro-2-fluoro-4-(hydroxymethyl)phenol (C4′)

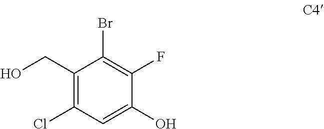 [SMILES: OCc1c(Cl)cc(O)c(F)c1Br]

To a solution of NaOH (6.2 g, 156.6 mmol) in H₂O (100 mL) at rt was added Intermediate A14′ (9.1 g, 40.4 mmol). The mixture was heated to 45° C. and HCHO (4.7 mL of 37% aqueous, 40.4 mmol) was added dropwise. The mixture was stirred at 45° C. overnight. The reaction was cooled to rt and diluted with water (50 mL), then acidified to pH˜-5-6 with 1N HCl and extracted with EtOAc (30 mL*3). The combined organic phase was washed with brine (100 mL), dried over Na₂SO₄ and concentrated in vacuo. The crude product was purified by silica gel column chromatography (pet. ether/EtOAc=50/1 to 5/1) to afford Intermediate C4′ (4.1 g, 39.7% yield) as a white solid.

TLC: EtOAc/pet. ether=1/5 (v/v), Rf=0.20

¹H NMR: (400 MHz, DMSO-d₆) δ 10.83 (s, 1H), 7.05 (d, J=7.8 Hz, 1H), 5.10 (t, J=5.2 Hz, 1H), 4.62 (d, J=5.1 Hz, 2H).

### Intermediate D1′

### Synthesis of ethyl 2-(2-fluoro-4-(hydroxymethyl)-5-(trifluoromethyl)phenoxy)acetate (D1′)

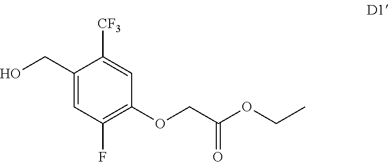 [SMILES: CCOC(=O)COc1cc(C(F)(F)F)c(CO)cc1F]

To a mixture of A5′ (300 mg, 1.02 mmol, 1.0 eq) in THE (5 mL) at 0° C. was added NaBH₄ (39 mg, 1.0 mmol, 1.0 eq). The mixture was stirred at rt for 1 h, then water (30 mL) was added. The mixture was extracted with EtOAc (25 mL*2); the combined organic layer was washed with water (25 mL*2), then brine (50 mL), dried over Na₂SO₄, and concentrated to dryness to afford product Intermediate D1′ (300 mg, 99.3% yield) as a white solid.

¹H NMR: (400 MHz, DMSO-d₆) δ 7.42-7.37 (m, 2H), 5.50 (t, J=5.8 Hz, 1H), 5.02 (s, 2H), 4.61 (d, J=5.7 Hz, 2H), 4.17 (q, J=7.1 Hz, 2H), 1.20 (t, J=7.1 Hz, 3H).

### Intermediate D2′

### Synthesis of ethyl 2-(3-chloro-2-fluoro-4-(hydroxymethyl)phenoxy)acetate (D2′)

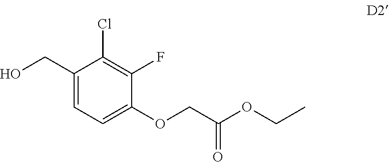 [SMILES: CCOC(=O)COc1ccc(CO)c(Cl)c1F]

To a solution of C1′ (250 mg, 1.42 mmol, 1.0 eq) in DMF (5 mL) at rt were added K₂CO₃ (235 mg, 1.70 mmol, 1.2 eq) and ethyl 2-bromoacetate (236 mg, 1.42 mmol, 1.0 eq). The mixture was stirred at rt overnight, then water (30 mL) was added and the mixture was extracted with EtOAc (5 mL*3). The combined organic phase was washed with brine (10 mL), dried over Na₂SO₄ and concentrated in vacuo to afford D2′ (300 mg, 80.6% yield) as a colorless oil.

TLC: EtOAc/pet. ether=1/3 (v/v), Rf=0.5

HNMR: ¹H NMR (400 MHz, DMSO-d₆) δ 7.24 (dd, J=8.8, 2.0 Hz, 1H), 7.12 (t, J=8.4 Hz, 1H), 5.36 (t, J=5.6 Hz, 1H), 4.91 (s, 2H), 4.50 (d, J=6.0 Hz, 2H), 4.16 (q, J=7.2 Hz, 2H), 1.20 (t, J=7.2 Hz, 3H).

### Intermediate D3′

### Synthesis of ethyl 2-(5-chloro-2-fluoro-4-(hydroxymethyl)phenoxy)acetate (D3′)

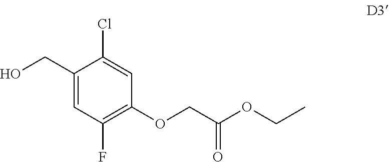 [SMILES: CCOC(=O)COc1cc(Cl)c(CO)cc1F]

To a solution of C2′ (250 mg, 1.42 mmol, 1.0 eq) in DMF (5 mL) at rt were added K₂CO₃ (235 mg, 1.70 mmol, 1.2 eq) and ethyl 2-bromoacetate (236 mg, 1.42 mmol, 1.0 eq). The mixture was stirred at rt overnight, then water (30 mL) was added and the mixture extracted with EtOAc (5 mL*3). The combined organic layers were washed with brine (10 mL), dried over Na₂SO₄ and concentrated in vacuo to afford Intermediate D3′ (350 mg, 94.1% yield) as a colorless oil.

TLC: EtOAc/pet. ether=1/3 (v/v), Rf=0.5

¹H NMR: (400 MHz, DMSO-d₆) δ 7.32 (d, J=12.0 Hz, 1H), 7.23 (d, J=8.0 Hz, 1H), 5.43 (t, J=5.6 Hz, 1H), 4.92 (s, 2H), 4.46 (d, J=5.6 Hz, 2H), 4.17 (q, J=6.8 Hz, 2H), 1.21 (t, J=7.2 Hz, 3H).

### Intermediate D4′

### Synthesis of ethyl 2-(5-bromo-2-fluoro-4-(hydroxymethyl)phenoxy)acetate (D4)

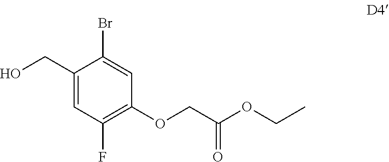 [SMILES: CCOC(=O)COc1cc(Br)c(CO)cc1F]

To a solution of C3′ (1.0 g, 4.5 mmol, 1.0 eq) in DMF (10 mL) at rt were added K₂CO₃ (938 mg, 6.79 mmol, 1.5 eq) and ethyl 2-bromoacetate (907 mg, 5.43 mmol, 1.2 eq). The mixture was stirred at rt for 2 h, then water (30 mL) was added, and the mixture extracted with EtOAc (20 mL*2). The combined organic layers were washed with water (20 mL*3) and brine (20 mL), dried over Na₂SO₄, and concentrated in vacuo to afford Intermediate D4′ (1.3 g, 93% yield) as a colorless oil.

TLC: Pet. ether/EtOAc=3/1 (v/v), Rf=0.56.

¹H NMR: (400 MHz, DMSO-d₆) δ 7.36 (d, J=8.0 Hz, 1H), 7.32 (d, J=12.0 Hz, 1H), 5.47 (t, J=5.6 Hz, 1H), 4.92 (s, 2H), 4.42 (d, J=5.2 Hz, 2H), 4.17 (q, J=7.2 Hz, 2H), 1.21 (t, J=7.2, 3H).

### Intermediate D5′

### Synthesis of ethyl 2-(3-chloro-2,6-difluoro-4-(hydroxymethyl)phenoxy)acetate (D5′)

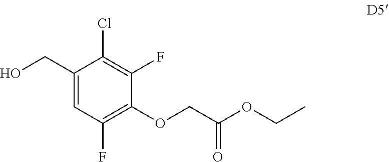 [SMILES: CCOC(=O)COc1c(F)cc(CO)c(Cl)c1F]

To a mixture of A12′ (200 mg, 718 μmol, 1.0 eq) in THE (2 mL) at 0° C. was added NaBH₄ (30 mg, 790 μmol, 1.1 eq); the mixture was stirred at rt for 1 h. Water (30 mL) was added, and the mixture was extracted with EtOAc (25 mL*2). The combined organic layers were washed with water (25 mL*2) and brine (50 mL), dried over Na₂SO₄, and concentrated to dryness to afford Intermediate D5′ (200 mg, 99.3% yield) as a white solid.

¹H NMR: (400 MHz, DMSO-d₆) δ 7.23 (dd, J=12.2, 2.3 Hz, 1H), 5.58 (t, J=5.7 Hz, 1H), 4.88 (s, 2H), 4.52 (d, J=5.7 Hz, 2H), 4.16 (q, J=7.1 Hz, 2H), 1.19 (t, J=7.1 Hz, 3H).

### Intermediate D6′

### Synthesis of ethyl 2-(3-bromo-5-chloro-2-fluoro-4-(hydroxymethyl)phenoxy)acetate (D6′)

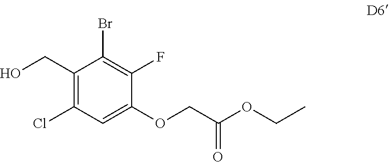 [SMILES: CCOC(=O)COc1cc(Cl)c(CO)c(Br)c1F]

To a solution of Intermediate C4′ (4.0 g, 15.7 mmol) in DMF (5 mL) at rt were added K₂CO₃ (2.6 g, 18.8 mmol) and ethyl 2-bromoacetate (2.6 g, 15.7 mmol); the mixture was stirred at rt for 1 h. The reaction was poured into water (50 mL); the mixture was extracted with EtOAc (20 mL*3). The combined organic phase was washed with brine (50 mL), dried over Na₂SO₄, and concentrated in vacuo to afford Intermediate D6′ (5.0 g, 93.5% yield) as a white solid.

TLC: EtOAc/pet. ether=1/5 (v/v), Rf=0.25

### Intermediate E1′

### Synthesis of ethyl 2-(4-(chloromethyl)-2-fluoro-5-(trifluoromethyl)phenoxy)acetate (E1)

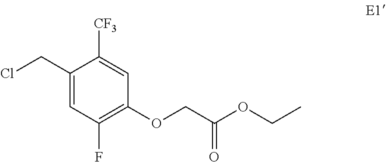 [SMILES: CCOC(=O)COc1cc(C(F)(F)F)c(CCl)cc1F]

A solution of D1′ (200 mg, 675 μmol, 1.0 eq) and SOCl₂ (120 mg, 1.01 mmol, 1.5 eq) in DCM (3 mL) was stirred at rt for 2 h. The mixture was concentrated to dryness to afford Intermediate E1′ (300 mg, 93.8% yield) as a colorless oil.

¹H NMR: (400 MHz, DMSO-d₆) δ 7.58 (dd, J=5.8, 2.1 Hz, 1H), 7.53 (dd, J=7.4, 2.2 Hz, 1H), 5.06 (s, 2H), 4.87 (s, 2H), 4.18 (q, J=7.1 Hz, 2H), 1.20 (t, J=7.1 Hz, 3H).

### Intermediate E2

### Synthesis of ethyl 2-(4-(bromomethyl)-3-chlorophenoxy)acetate (E2′)

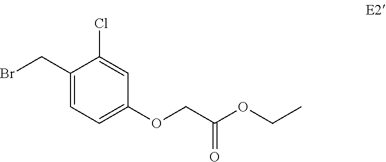 [SMILES: CCOC(=O)COc1ccc(CBr)c(Cl)c1]

To a solution of A6′ (1.0 g, 4.4 mmol, 1.0 eq) in CCl₄ (10 mL) at rt were added N-bromosuccinimide (778 mg, 4.37 mmol, 1.0 eq) and AIBN (140 mg, 0.87 mmol, 0.2 eq). The reaction was stirred at 80° C. for 1.5 h; the mixture was cooled to rt and filtered. The filtrate was diluted with DCM (30 mL), washed with brine (30 mL), dried over Na₂SO₄, and concentrated in vacuo. The residue was purified by silica gel column chromatography (pet. ether/EtOAc=100/1 to 40/1) to afford Intermediate E2′ (750 mg, 57.7% yield) as a yellow solid.

TLC: Pet. ether/EtOAc=5/1 (v/v), Rf=0.50

¹H NMR: (400 MHz, DMSO-d₆) δ 7.53 (d, J=8.8 Hz, 1H), 7.10 (d, J=2.8 Hz, 1H), 6.94 (dd, J=8.4, 2.8 Hz, 1H), 4.85 (s, 2H), 4.72 (s, 2H), 4.17 (q, J=7.2 Hz, 2H), 1.21 (t, J=7.2 Hz, 3H).

### Intermediate E3′

### Synthesis of ethyl 2-(3-bromo-4-(bromomethyl)phenoxy)acetate (E3′)

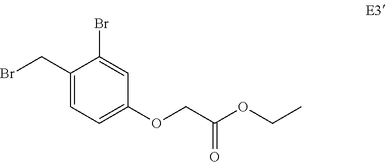 [SMILES: CCOC(=O)COc1ccc(CBr)c(Br)c1]

To a solution of A7′ (6.3 g, 23 mmol, 1.0 eq) in CCl₄ (70 mL) at rt were added benzoyl peroxide (279 mg, 1.15 mmol, 0.05 eq) and N-bromosuccinimide (4.1 g, 23 mmol, 1.0 eq). The mixture was stirred at 100° C. for 1 h, then the mixture was cooled to rt and filtered. The filtrate was diluted with DCM (50 mL), washed with brine (30 mL), dried over Na₂SO₄ and concentrated in vacuo. The residue was purified by silica gel column chromatography (pet. ether/EtOAc=100/1 to 20/1) to afford Intermediate E3′ (5.5 g, 68% yield) as a white solid.

TLC: Pet. ether/EtOAc=3/1 (v/v), Rf=0.37

¹H NMR: (400 MHz, DMSO-d₆) δ 7.55 (d, J=8.4 Hz, 1H), 7.24 (d, J=2.4 Hz, 1H), 6.98 (dd, J=8.8, 2.8 Hz, 1H), 4.86 (s, 2H), 4.72 (s, 2H), 4.19-4.14 (m, 2H), 1.21 (t, J=7.2 Hz, 3H).

### Intermediate E4′

### Synthesis of ethyl 2-(3-chloro-4-(chloromethyl)-2-fluorophenoxy)acetate (E4′)

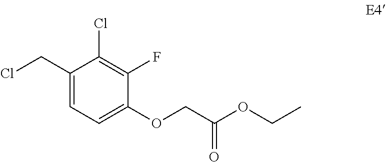 [SMILES: CCOC(=O)COc1ccc(CCl)c(Cl)c1F]

To a solution of D2′ (330 mg, 1.26 mmol, 1.0 eq) in DCM (5 mL) at 0° C. was added SOCl₂ (179 mg, 1.51 mmol, 1.2 eq). The mixture was stirred at rt for 3 h, then diluted with DCM (10 mL), and concentrated in vacuo to afford Intermediate E4′ (300 mg, 84.9% yield) as a colorless oil.

TLC: DCM/MeOH=10/1 (v/v), Rf=0.7.

### Intermediate E5′

### Synthesis of ethyl 2-(5-chloro-4-(chloromethyl)-2-fluorophenoxy)acetate (E5′)

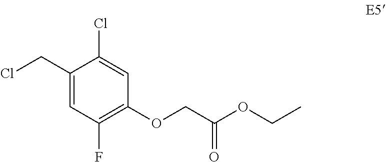 [SMILES: CCOC(=O)COc1cc(Cl)c(CCl)cc1F]

To a solution of D3′ (200 mg, 761 μmol, 1.0 eq) in DCM (5 mL) at rt was added SOCl₂ (108 mg, 913 μmol, 1.2 eq). The mixture was stirred at rt for 2 h, then concentrated in vacuo to afford Intermediate E5′ (200 mg, 93.4% yield) as a colorless oil.

TLC: DCM/MeOH=10/1 (v/v), Rf=0.7

¹H NMR: (400 MHz, DMSO-d₆) δ 7.58 (d, J=11.6 Hz, 1H), 7.35 (d, J=7.6 Hz, 1H), 4.97 (s, 2H), 4.75 (s, 2H), 4.22-4.13 (m, 2H), 1.21 (td, J=6.8, 0.8 Hz, 3H).

### Intermediate E6′

### Synthesis of ethyl 2-(5-bromo-4-(chloromethyl)-2-fluorophenoxy)acetate (E6′)

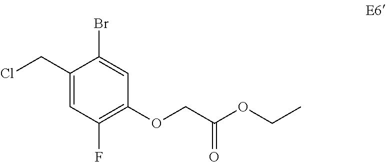 [SMILES: CCOC(=O)COc1cc(Br)c(CCl)cc1F]

To a solution of D4′ (1.2 g, 3.91 mmol, 1.0 eq) in DCM (6 mL) at 0° C. was added SOCl₂ (697 mg, 5.86 mmol, 1.5 eq). The mixture was stirred at rt for 2 h, then was concentrated in vacuo to afford intermediate E6′ (1.2 g, 92% yield) as a colorless oil.

TLC: Pet. ether/EtOAc=3/1 (v/v), Rf=0.83

¹H NMR: (400 MHz, DMSO-d₆) δ 7.59 (d, J=11.6 Hz, 1H), 7.47 (d, J=8.0 Hz, 1H), 4.98 (s, 2H), 4.74 (s, 2H), 4.18 (q, J=7.2 Hz, 2H), 1.21 (t, J=7.2 Hz, 3H).

### Intermediate E7′

### Synthesis of ethyl 2-(3-chloro-4-(chloromethyl)-2,6-difluorophenoxy)acetate (E7′)

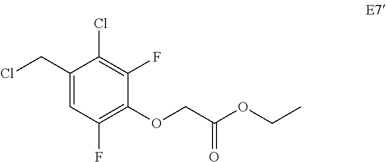 [SMILES: CCOC(=O)COc1c(F)cc(CCl)c(Cl)c1F]

A solution of D5′ (300 mg, 1.07 mmol, 1.0 eq) and SOCl₂ (191 mg, 1.60 mmol, 1.5 eq) in DCM (3 mL) was stirred at rt for 2 h. The mixture was concentrated to dryness to afford Intermediate E7′ (300 mg, 93.8% yield) as a colorless oil.

### Intermediate E8′

### Synthesis of ethyl 2-(3-bromo-5-chloro-4-(chloromethyl)-2-fluorophenoxy)acetate (E8′)

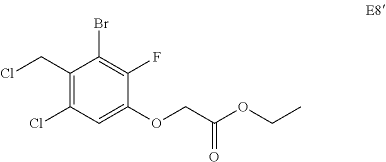 [SMILES: CCOC(=O)COc1cc(Cl)c(CCl)c(Br)c1F]

To a solution of Intermediate D6′ (5.0 g, 14.6 mmol) in DCM (50 mL) was added SOCl₂ (2.6 g, 21.9 mmol). The mixture was stirred at rt for 1 h. Water (50 mL) was added and the resultant mixture was extracted with DCM (20 mL*3). The combined organic phase was washed with brine (50 mL), dried over Na₂SO₄ and concentrated in vacuo to afford Intermediate E8′ (5.0 g, 94.9% yield) as a white solid.

TLC: EtOAc/pet. ether=1/5 (v/v), Rf=0.5

### Intermediate F1′

### Synthesis of ethyl 2-(3-bromo-5-chloro-2-fluoro-4-(2-fluoro-4-hydroxy-3-isopropylbenzyl)phenoxy)acetate (F1′)

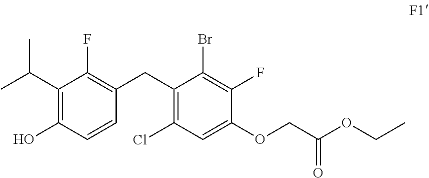 [SMILES: CCOC(=O)COc1cc(Cl)c(Cc2ccc(O)c(C(C)C)c2F)c(Br)c1F]

To a solution of Intermediate E8′ (500 mg, 1.4 mmol) in DCE (10 mL) at rt were added Intermediate B2 (646 g, 4.2 mmol) and ZnCl₂ (3.5 mmol, 475 mg). The reaction was heated to 85° C. and stirred overnight. The reaction was cooled to rt; water (30 mL) was added and the mixture was extracted with DCM (20 mL*3). The combined organic phase was washed with brine (50 mL), dried over Na₂SO₄ and concentrated in vacuo. The crude product was purified by silica gel column chromatography (EtOAc/pet. ether=1/100 to 1/10) to afford Intermediate F1′(260 mg, 39.2% yield) as a white solid.

TLC: Pet. ether/EtOAc=5/1 (v/v), Rf=0.20

¹H NMR: (400 MHz, DMSO-d₆) δ 9.55 (d, J=1.6 Hz, 1H), 7.47 (d, J=7.6 Hz, 1H), 6.48 (dd, J=8.4, 1.2 Hz, 1H), 6.23 (t, J=8.4 Hz, 1H), 5.01 (s, 2H), 4.19 (d, J=7.2 Hz, 2H), 4.09 (s, 2H), 3.42-3.35 (m, 1H), 1.30-1.23 (m, 9H).

### Intermediate F2′

### Synthesis of ethyl 2-(5-chloro-2-fluoro-4-(2-fluoro-4-hydroxy-3-isopropylbenzyl)-3-(prop-1-en-2-yl)phenoxy)acetate (F2′)

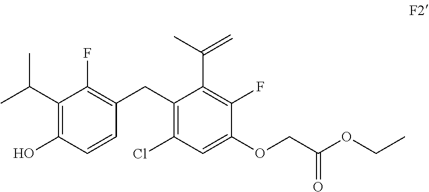 [SMILES: C=C(C)c1c(F)c(OCC(=O)OCC)cc(Cl)c1Cc1ccc(O)c(C(C)C)c1F]

To a mixture of Intermediate F1′ (260 mg, 544 μmol), and potassium propenyl-2-boron(trifluoride) (161 mg, 1.1 mmol) in 1,4-dioxane (5.0 mL) and water (0.2 mL) at rt were added Pd(dppf)Cl₂—CH₂Cl₂ (44 mg, 54 μmol) and Cs₂CO₃ (355 mg, 1.1 mmol) under N₂ (g). The mixture was heated to 120° C. for 2 h in a sealed tube. The mixture was cooled to rt. Water (30 mL) was added and the mixture was extracted with EtOAc (15 mL*3). The combined organic phase was washed with brine (30 mL), dried over Na₂SO₄, filtered and concentrated in vacuo. Purification by Prep-TLC (EtOAc/pet. ether=1/5) afforded Intermediate F2′ (90 mg, 37.7% yield) as a white solid.

TLC: Pet. ether/EtOAc=5/1 (v/v), Rf=0.23

¹H NMR: (400 MHz, DMSO-d₆) δ 9.49 (d, J=1.2 Hz, 1H), 7.26 (d, J=8.0 Hz, 1H), 6.47 (d, J=8.4 Hz, 1H), 6.22 (t, J=8.4 Hz, 1H), 5.29-5.25 (m, 1H), 4.95 (s, 2H), 4.78-4.75 (m, 1H), 4.18 (t, J=7.2 Hz, 2H), 3.85 (s, 2H), 3.40-3.36 (m, 1H), 1.78 (t, J=1.2 Hz, 3H), 1.24 (d, J=1.2 Hz, 9H).

### Example 1

### Synthesis of ethyl 2-((7-(2-fluoro-4-hydroxy-3-isopropylbenzyl)-2,3-dihydro-1H-inden-4-yl)oxy)acetate (Compound 1)

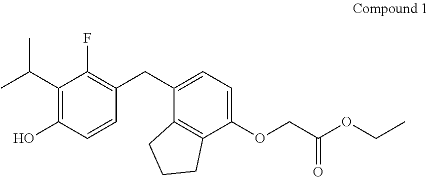 [SMILES: CCOC(=O)COc1ccc(Cc2ccc(O)c(C(C)C)c2F)c2c1CCC2]

To a solution of Intermediate E1 (110 mg, 0.41 mmol, 1.0 eq) and Intermediate B2 (126 mg, 0.82 mmol) in DCE (5 mL) was added ZnCl₂ (1 Min THF, 1.02 ml, 1.02 mmol, 2.5 eq) and the mixture was stirred at 85° C. overnight. The mixture was concentrated to dryness, H₂O (30 mL) was added, and the mixture was extracted with EtOAc (25 mL*2). The combined organic layer was washed with brine (50 mL), dried over Na₂SO₄, and concentrated to dryness. The crude material was purified by Prep-TLC (pet. ether/EtOAc=4/1) to afford Compound 1 (50 mg, 32% yield) as a white solid.

TLC: EtOAc/pet. ether=1/5 (v/v), Rf=0.31.

### Example 2

### Synthesis of 2-((7-(2-fluoro-4-hydroxy-3-isopropylbenzyl)-2,3-dihydro-1H-inden-4-yl)oxy)acetic acid (Compound 2)

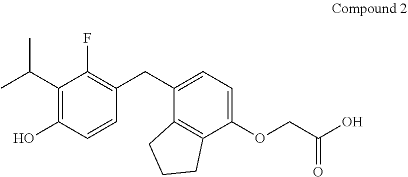 [SMILES: CC(C)c1c(O)ccc(Cc2ccc(OCC(=O)O)c3c2CCC3)c1F]

To a solution of Compound 1 (50 mg, 130 μmol, 1.0 eq) in MeOH/H₂O (3 mL/1 mL) at rt was added LiOH·H₂O (9 mg, 388 μmol, 3.0 eq) and the mixture was stirred 1 h. The mixture was diluted with water (10 mL), acidified to pH=3-4 with 1 N HCl and extracted with EtOAc (15 mL*2). The combined organic phase was washed with brine (30 mL), dried over Na₂SO₄ and concentrated under reduced pressure. The crude product was purified by Prep-HPLC to afford Compound 2 (16 mg, 35% yield) as a white solid.

TLC: MeOH/DCM=1/10 (v/v), Rf=0.30

LCMS: T=3.924 min, [M−1]=357.1

¹H NMR: (400 MHz, DMSO-d₆) δ 12.87 (s, 1H), 9.47 (s, 1H), 6.79 (d, J=8.2 Hz, 1H), 6.69 (t, J=8.6 Hz, 1H), 6.53 (t, J=9.0 Hz, 2H), 4.62 (s, 2H), 3.69 (s, 2H), 3.35-3.33 (m, 1H), 2.78 (dt, J=11.8, 7.5 Hz, 4H), 1.98 (p, J=7.5 Hz, 2H), 1.23 (d, J=7.0 Hz, 6H).

¹⁹F NMR: (376 MHz, DMSO-d₆) δ −120.51

### Example 3

### Synthesis of ethyl 2-((7-(2-fluoro-4-hydroxy-3-isopropylbenzyl)-1-methyl-2,3-dihydro-1H-inden-4-yl)oxy)acetate (Compound 3)

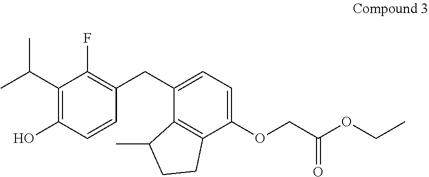 [SMILES: CCOC(=O)COc1ccc(Cc2ccc(O)c(C(C)C)c2F)c2c1CCC2C]

To a solution of Intermediate E2 (35 mg, 0.12 mmol, 1.0 eq) and Intermediate B2 (38 mg, 0.25 mmol, 2.0 eq) in DCE (3 mL) was added ZnCl₂ (1 M, 0.31 mL, 0.31 mmol, 2.5 eq) and the mixture was stirred at 85° C. overnight. The mixture was concentrated to dryness, H₂O (5 mL) was added and the mixture was extracted with EtOAc (5 mL*2). The combined organic layer was washed with brine (10 mL), dried over Na₂SO₄, and concentrated to dryness. The residue was purified by Prep-TLC to afford Compound 3 (20 mg, 40% yield) as a white solid.

LCMS: T=2.436 min, [M−1]=399.2.

### Example 4

### Synthesis of 2-((7-(2-fluoro-4-hydroxy-3-isopropylbenzyl)-1-methyl-2,3-dihydro-1H-inden-4-yl)oxy)acetic acid (Compound 4)

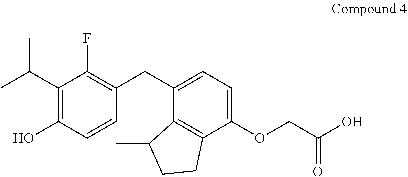 [SMILES: CC(C)c1c(O)ccc(Cc2ccc(OCC(=O)O)c3c2C(C)CC3)c1F]

To a solution of Compound 3 (20 mg, 50 μmol, 1.0 eq) in MeOH/H₂O (2 mL/0.3 mL) at rt was added NaOH (6.0 mg, 150 μmol, 3.0 eq). The mixture was stirred for 1 h then was diluted with water (10 mL), acidified to pH=3-4 with 1 N HCl and extracted with EtOAc (15 mL*2). The combined organic phase was washed with brine (10 mL), dried over Na₂SO₄ and concentrated under reduced pressure. The crude product was purified by Prep-HPLC to afford Compound 4 (7.0 mg, 36% yield) as a white solid.

LCMS: T=2.339 min, [M−1]=371.1

¹H NMR: (400 MHz, DMSO-d₆) δ 9.48 (s, 1H), 6.73-6.64 (m, 2H), 6.53 (dd, J=8.4, 3.5 Hz, 2H), 4.61 (s, 2H), 3.80-3.70 (m, 2H), 3.41-3.39 (m, 1H), 3.27-3.24 (m, 1H), 2.89-2.72 (m, 2H), 2.15-2.07 (m, 1H), 1.75-1.67 (m, 1H), 1.24 (d, J=7.0 Hz, 6H), 1.05 (d, J=7.0 Hz, 3H) ¹⁹F NMR: (376 MHz, DMSO-d₆) δ −120.62.

### Example 5

### Synthesis of ethyl 2-((7-(4-hydroxy-3-isopropylbenzyl)-6-methyl-2,3-dihydro-1H-inden-4-yl)oxy)acetate (Compound 5)

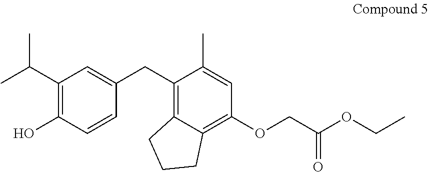 [SMILES: CCOC(=O)COc1cc(C)c(Cc2ccc(O)c(C(C)C)c2)c2c1CCC2]

To a solution of Intermediate E3 (200 mg, 0.71 mmol, 1.0 eq) and 2-isopropylphenol (193 mg, 1.41 mmol, 2.0 eq) in DCE (5 mL) was added ZnCl₂ (1 M, 1.77 mL, 1.77 mmol, 2.5 eq) and the mixture was stirred at 85° C. overnight. The mixture was concentrated to dryness, H₂O (30 mL) was added and the mixture was extracted with EtOAc (25 mL*2). The combined organic layer was washed with brine (50 mL), dried over Na₂SO₄, concentrated to dryness, and purified by RP column to afford Compound 5 (150 mg, 55% yield) as a white solid.

TLC: EtOAc/pet. ether=1/5 (v/v), Rf=0.31.

### Example 6

### Synthesis of 2-((7-(4-hydroxy-3-isopropylbenzyl)-6-methyl-2,3-dihydro-1H-inden-4-yl)oxy)acetic acid (Compound 6)

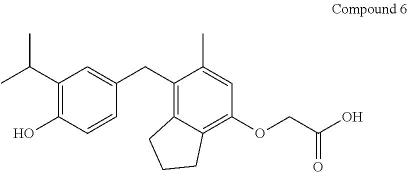 [SMILES: Cc1cc(OCC(=O)O)c2c(c1Cc1ccc(O)c(C(C)C)c1)CCC2]

To a solution of Compound 5 (200 mg, 523 μmol, 1.0 eq) in MeOH/H₂O (5 mL/1 mL) at rt was added NaOH (42 mg, 1.1 mmol, 2.0 eq). The mixture was stirred at rt for 1 h, then the mixture was diluted with water (10 mL), acidified to pH=3-4 with 1 N HCl and extracted with EtOAc (15 mL*2). The combined organic phase was washed with brine (30 mL), dried over Na₂SO₄, and concentrated in vacuo. The crude product was purified by Prep-HPLC to afford Compound 6 (30 mg, 16% yield) as a white solid.

TLC: MeOH/DCM=1/10 (v/v), Rf=0.30

LCMS: T=1.920 min, [M−1]=353.2

¹H NMR: (400 MHz, DMSO-d₆) δ 12.86 (s, 1H), 8.97 (s, 1H), 6.88 (d, J=2.2 Hz, 1H), 6.60 (d, J=8.1 Hz, 1H), 6.52 (d, J=2.2 Hz, 1H), 6.47 (s, 1H), 4.62 (s, 2H), 3.74 (s, 2H), 3.12 (p, J=6.9 Hz, 1H), 2.77 (q, J=7.9 Hz, 4H), 2.14 (s, 3H), 1.97 (q, J=7.4 Hz, 2H), 1.10 (d, J=6.9 Hz, 6H).

### Example 7

### Synthesis of ethyl 2-((7-(3-(1-(4-fluorophenyl)ethyl)-4-hydroxybenzyl)-6-methyl-2,3-dihydro-1H-inden-4-yl)oxy)acetate (Compound 7)

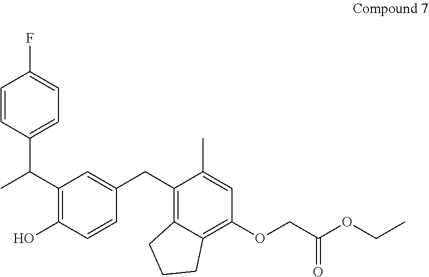 [SMILES: CCOC(=O)COc1cc(C)c(Cc2ccc(O)c(C(C)c3ccc(F)cc3)c2)c2c1CCC2]

To a solution of Intermediate E3 (200 mg, 0.71 mmol, 1.0 eq) and Intermediate B4 (193 mg, 1.41 mmol, 3.0 eq) in DCE (5 mL) was added ZnCl₂ (1 M, 1.77 mL, 1.77 mmol, 2.5 eq) and the mixture was stirred at 85° C. overnight. The mixture was concentrated to dryness, H₂O (30 mL) was added, and the mixture was extracted with EtOAc (25 mL*2). The combined organic layer was washed with brine (50 mL), dried over Na₂SO₄, concentrated to dryness, and purified by RP column chromatography to afford Compound 7 (150 mg, 46% yield) as a white solid.

TLC: EtOAc/pet. ether=1/5 (v/v), Rf=0.29.

### Example 8

### Synthesis of 2-((7-(3-(1-(4-fluorophenyl)ethyl)-4-hydroxybenzyl)-6-methyl-2,3-dihydro-1H-inden-4-yl)oxy)acetic acid (Compound 8)

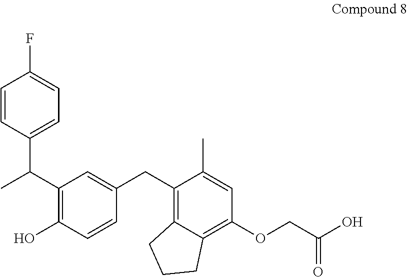 [SMILES: Cc1cc(OCC(=O)O)c2c(c1Cc1ccc(O)c(C(C)c3ccc(F)cc3)c1)CCC2]

To a solution of Compound 7 (150 mg, 324 μmol) in MeOH/H₂O (3 mL/1 mL) at rt was added NaOH (39 mg, 970 μmol) and the mixture was stirred at rt for 1 h. The mixture was diluted with water (10 mL), acidified to pH=3-4 with 1 N HCl, and extracted with EtOAc (15 mL*2). The combined organic phase was washed with brine (30 mL), dried over Na₂SO₄, and concentrated under reduced pressure. The crude product was purified by Prep-HPLC to afford Compound 8 (35 mg, 25% yield) as a white solid.

TLC: MeOH/DCM=1/10 (v/v), Rf=0.30

LCMS: T=2.255 min, [M−1]=433.2

¹H NMR: (400 MHz, DMSO-d₆) δ 12.89 (s, 1H), 9.11 (s, 1H), 7.22-7.13 (m, 2H), 7.10-7.00 (m, 2H), 6.81 (d, J=2.1 Hz, 1H), 6.62 (d, J=8.2 Hz, 1H), 6.56 (dd, J=8.2, 2.1 Hz, 1H), 6.45 (s, 1H), 4.62 (s, 2H), 4.37 (q, J=7.1 Hz, 1H), 3.71 (s, 2H), 2.77 (t, J=7.4 Hz, 2H), 2.69 (t, J=7.5 Hz, 2H), 2.11 (s, 3H), 1.93 (p, J=7.4 Hz, 2H), 1.44 (d, J=7.3 Hz, 3H)

¹⁸F NMR: (376 MHz, DMSO-d₆) δ −117.89.

### Example 9

### Synthesis of ethyl 2-((7-(2-fluoro-4-hydroxy-3-isopropylbenzyl)-6-methyl-2,3-dihydro-1H-inden-4-yl)oxy)acetate (Compound 9)

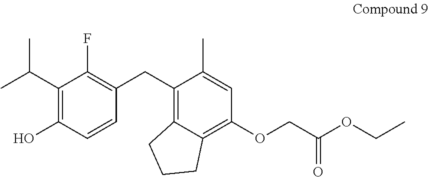 [SMILES: CCOC(=O)COc1cc(C)c(Cc2ccc(O)c(C(C)C)c2F)c2c1CCC2]

To a solution of Intermediate E3 (200 mg, 0.71 mmol, 1.0 eq) and Intermediate B2 (327 mg, 2.12 mmol, 3.0 eq) in DCE (5 mL) was added ZnCl₂ (1 M, 1.77 mL, 1.77 mmol, 2.5 eq) and the mixture was stirred at 85° C. overnight. The mixture was concentrated to dryness, H₂O (30 mL) was added, and the mixture was extracted with EtOAc (25 mL*2). The combined organic layer was washed with brine (50 mL), dried over Na₂SO₄, and concentrated in vacuo. The residue was purified by RP column chromatography to afford product Compound 9 (200 mg, 71% yield) as a white solid.

TLC: EtOAc/pet. ether=1/5 (v/v), Rf=0.31.

### Example 10

### Synthesis of 2-((7-(2-fluoro-4-hydroxy-3-isopropylbenzyl)-6-methyl-2,3-dihydro-1H-inden-4-yl)oxy)acetic acid (Compound 10)

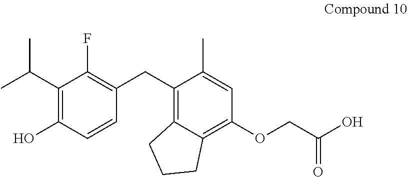 [SMILES: Cc1cc(OCC(=O)O)c2c(c1Cc1ccc(O)c(C(C)C)c1F)CCC2]

To a solution of Compound 9 (200 mg, 0.5 mmol, 1.0 eq) in MeOH/H₂O (3 mL/1 mL) at rt was added NaOH (60 mg, 1.50 mmol, 3.0 eq) and the mixture was stirred for 1 h. The mixture was diluted with water (10 mL), acidified to pH=3-4 with 1 N HCl, and extracted with EtOAc (15 mL*2). The combined organic phase was washed with brine (30 mL), dried over Na₂SO₄ and concentrated in vacuo. The crude product was purified by Prep-HPLC to afford Compound 10 (35 mg, 19% yield) as a white solid.

TLC: MeOH/DCM=1/10 (v/v), Rf=0.30

LCMS: T=2.321 min, [M−1]=371.2

¹H NMR: (400 MHz, DMSO-d₆) δ 12.84 (s, 1H), 9.42 (d, J=1.4 Hz, 1H), 6.49 (s, 1H), 6.44 (d, J=8.4 Hz, 1H), 6.25 (t, J=8.6 Hz, 1H), 4.64 (s, 2H), 3.71 (s, 2H), 3.38 (d, J=7.0 Hz, 1H), 2.79 (t, J=7.5 Hz, 2H), 2.71 (t, J=7.5 Hz, 2H), 2.10 (s, 3H), 1.96 (p, J=7.6 Hz, 2H), 1.25 (d, J=6.6 Hz, 6H)

¹⁹F NMR: (376 MHz, DMSO-d₆) δ −120.59.

### Example 11

### Synthesis of 2-((7-(2-fluoro-4-hydroxy-3-isopropylbenzyl)-6-methyl-2,3-dihydro-1H-inden-4-yl)oxy)-N-methylacetamide (Compound 11)

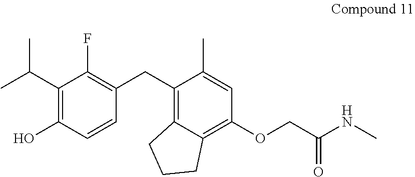 [SMILES: CNC(=O)COc1cc(C)c(Cc2ccc(O)c(C(C)C)c2F)c2c1CCC2]

To a solution of Compound 10 (20 mg, 54 μmol, 1.0 eq) in DCM (2 mL) at rt was added (COCl)₂ (10 mg, 81 μmol, 1.5 eq) and 1 drop of DMF. The mixture was stirred for 1 h, then concentrated to dryness, and dissolved in DCM (2 mL). The mixture was added to a solution of methylamine (2 M in THF, 81 μL, 161 μmol, 3.0 eq) and stirred for 10 min. The mixture was diluted with water (10 mL) and extracted with DCM (5 mL). The organic phase was washed with brine (30 mL), dried over Na₂SO₄, and concentrated in vacuo. The crude product was purified by Prep-HPLC to afford Compound 11 (5.0 mg, 25% yield) as a white solid.

TLC: MeOH/DCM=1/10 (v/v), Rf=0.64

LCMS: T=2.396 min, [M−1]=384.2

¹H NMR: (400 MHz, DMSO-d₆) δ 9.42 (d, J=1.4 Hz, 1H), 7.82 (d, J=5.8 Hz, 1H), 6.54 (s, 1H), 6.43 (d, J=8.8 Hz, 1H), 6.25 (t, J=8.6 Hz, 1H), 4.43 (s, 2H), 3.71 (s, 2H), 3.40 (d, J=7.5 Hz, 1H), 2.86 (t, J=7.5 Hz, 2H), 2.72 (d, J=7.5 Hz, 2H), 2.66 (d, J=4.7 Hz, 3H), 2.10 (s, 3H), 1.96 (p, J=7.5 Hz, 2H), 1.25 (d, J=7.0 Hz, 6H)

¹⁹F NMR: (376 MHz, DMSO-d₆) δ −120.56.

### Example 12

### Synthesis of 2-((7-(2-fluoro-4-hydroxy-3-isopropylbenzyl)-6-methyl-2,3-dihydro-1H-inden-4-yl)oxy)-N,N-dimethylacetamide (Compound 12)

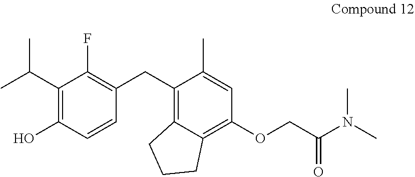 [SMILES: Cc1cc(OCC(=O)N(C)C)c2c(c1Cc1ccc(O)c(C(C)C)c1F)CCC2]

To a solution of Compound 10 (70 mg, 188 μmol, 1.0 eq) in DCM (2 mL) at rt was added (COCl)₂ (36 mg, 280 μmol, 1.5 eq) and 1 drop of DMF. The mixture was stirred for 1 h then concentrated to dryness, and dissolved in DCM (2 mL). The mixture was added to a solution of dimethylamine (1 M in THF, 1.41 mL, 1.41 mmol, 5.0 eq) and stirred for 10 min.

The mixture was diluted with water (10 mL) and extracted with DCM (5 mL). The organic phase was washed with brine (30 mL), dried over Na₂SO₄, and concentrated in vacuo. The crude product was purified by Prep-HPLC to afford Compound 12 (16 mg, 22% yield) as a white solid.

TLC: MeOH/DCM=1/10 (v/v), Rf=0.64

LCMS: T=2.535 min, [M−1]=398.2

¹H NMR: (400 MHz, DMSO-d₆) δ 9.41 (s, 1H), 6.53 (s, 1H), 6.44 (d, J=8.3 Hz, 1H), 6.26 (t, J=8.6 Hz, 1H), 4.74 (s, 2H), 3.71 (s, 2H), 3.41-3.39 (m, 1H), 3.00 (s, 3H), 2.84 (s, 3H), 2.79 (t, J=7.4 Hz, 2H), 2.70 (t, J=7.4 Hz, 2H), 2.10 (s, 3H), 1.96 (p, J=7.5 Hz, 2H), 1.31-1.20 (m, 6H)

¹⁹F NMR: (376 MHz, DMSO-d₆) δ −120.60.

### Example 13

### Synthesis of ethyl 2-((4-(4-hydroxy-3-isopropylbenzyl)naphthalen-1-yl)oxy)acetate (Compound 13)

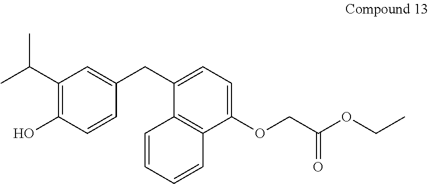 [SMILES: CCOC(=O)COc1ccc(Cc2ccc(O)c(C(C)C)c2)c2ccccc12]

To a solution of Intermediate F1 (70 mg, 160 μmol, 1.0 eq) in DCM (14 mL) at rt was added TFA (37 μL, 480 μmol, 3.0 eq) and Et₃SiH (127 μL, 798 μmol, 5.0 eq) and the reaction was heated at 50° C. overnight. After cooling to rt, the reaction mixture was diluted with DCM (20 mL), washed brine (10 mL*2), dried over Na₂SO₄, and concentrated in vacuo. The crude product was purified through Prep-TLC (EtOAc/pet. ether=1/3) to afford Compound 13 (60 mg, 99% yield) as a light yellow oil.

TLC: EtOAc/pet. ether=1/3 (v/v), Rf=0.6

¹H NMR: (400 MHz, DMSO-d₆) δ 9.01 (s, 1H), 8.27-8.21 (m, 1H), 8.04-7.99 (m, 1H), 7.55-7.46 (m, 2H), 7.20 (d, J=8.0 Hz, 1H), 7.04 (s, 1H), 6.83 (d, J=8.0 Hz, 1H), 6.72 (dd, J=8.4, 2.4 Hz, 1H), 6.62 (d, J=8.4 Hz, 1H), 4.95 (s, 2H), 4.23-4.16 (m, 4H), 3.12 (p, J=6.8 Hz, 1H), 1.23 (s, 3H), 1.10 (d, J=7.2 Hz, 6H).

### Example 14

### Synthesis of 2-((4-(4-hydroxy-3-isopropylbenzyl)naphthalen-1-yl)oxy)acetic acid (Compound 14)

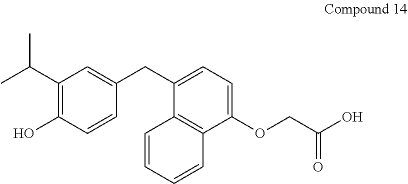 [SMILES: CC(C)c1cc(Cc2ccc(OCC(=O)O)c3ccccc23)ccc1O]

To a solution of Compound 13 (55 mg, 150 μmol, 1.0 eq) in THF/H₂O (2 mL/0.5 mL) was added LiOH·H₂O (12 mg, 290 μmol, 2.0 eq). The mixture was stirred at rt overnight, diluted with water (10 mL), acidified to pH=3-4 with 1N HCl, and extracted with EtOAc (10 mL*2). The combined organic phase was washed with brine (10 mL), dried over Na₂SO₄, and concentrated in vacuo. The crude product was purified by Prep-HPLC to afford Compound 14 (20 mg, 39% yield) as a white solid.

TLC: MeOH/DCM=1/10 (v/v), Rf=0.2

LCMS: T=3.310 min, [M−1]=349.0

¹H NMR: (400 MHz, DMSO-d₆) δ 13.08 (s, 1H), 9.02 (s, 1H), 8.28-8.21 (m, 1H), 8.01 (d, J=8.0 Hz, 1H), 7.55-7.46 (m, 2H), 7.20 (d, J=7.6 Hz, 1H), 7.05 (s, 1H), 6.82 (d, J=8.0 Hz, 1H), 6.72 (d, J=8.4 Hz, 1H), 6.62 (d, J=8.0 Hz, 1H), 4.85 (s, 2H), 4.20 (s, 2H), 3.13 (p, J=6.8 Hz, 1H), 1.10 (d, J=6.8 Hz, 6H).

### Example 15

### Synthesis of ethyl 2-((5-fluoro-7-(4-hydroxy-3-isopropylbenzyl)-2,3-dihydro-1H-inden-4-yl)oxy)acetate (Compound 15)

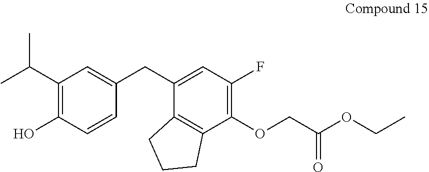 [SMILES: CCOC(=O)COc1c(F)cc(Cc2ccc(O)c(C(C)C)c2)c2c1CCC2]

To a solution of 2-isopropylphenol (71 mg, 0.51 mmol, 3.0 eq) and Intermediate E4 (50 mg, 0.17 mmol, 1.0 eq) in DCE (5 mL) at rt was added ZnCl₂ (1 Min THF, 350 μL, 0.35 mmol, 2.0 eq). The mixture was then heated to 85° C. and stirred overnight. The mixture was concentrated under reduce pressure and purified through Prep-TLC (EtOAc/pet. ether=1/5) to afford Compound 15 (35 mg, 52% yield) as colorless oil.

¹H NMR: (400 MHz, DMSO-d₆) δ 9.05 (s, 1H), 6.94 (s, 1H), 6.74 (d, J=12.7 Hz, 2H), 6.69-6.63 (m, 1H), 4.69 (d, J=2.4 Hz, 2H), 4.19-4.08 (m, 2H), 3.69 (d, J=2.5 Hz, 2H), 3.20-3.08 (m, 1H), 2.89 (t, J=8.0 Hz, 2H), 2.74 (t, J=7.5 Hz, 2H), 2.00 (d, J=10.6 Hz, 2H), 1.18 (ddd, J=8.2, 6.7, 2.5 Hz, 3H), 1.12 (dd, J=7.1, 2.5 Hz, 6H).

### Example 16

### Synthesis of 2-((5-fluoro-7-(4-hydroxy-3-isopropylbenzyl)-2,3-dihydro-1H-inden-4-yl)oxy)acetic acid (Compound 16)

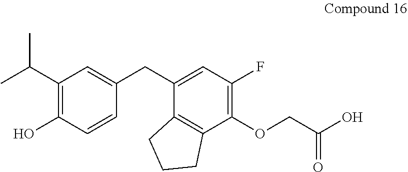 [SMILES: CC(C)c1cc(Cc2cc(F)c(OCC(=O)O)c3c2CCC3)ccc1O]

To a solution of Compound 15 (35 mg, 90 μmol, 1.0 eq) in THF/water (3 mL/1 mL) at rt was added LiOH·H₂O (6 mg, 136 μmol, 1.5 eq). After 2 h the reaction was acidified to pH=5 with aqueous HCl (0.5 N), and extracted with EtOAc (10 mL*2). The combined organic layer was dried over Na₂SO₄ and concentrated in vacuo to give the crude product that was purified by Prep-TLC (methanol/DCM=1/10) to afford Compound 16 (25 mg, 70% yield, 91% purity) as a white solid.

LCMS: T=1.71 min; [M−1]=357.1

¹H NMR: (400 MHz, DMSO-d₆) δ 12.86 (s, 1H), 9.04 (s, 1H), 6.95 (d, J=2.2 Hz, 1H), 6.78-6.70 (m, 2H), 6.66 (d, J=8.1 Hz, 1H), 4.60 (s, 2H), 3.69 (s, 2H), 3.15 (p, J=7.0 Hz, 1H), 2.89 (t, J=7.5 Hz, 2H), 2.74 (t, J=7.5 Hz, 2H), 1.99 (p, J=7.5 Hz, 2H), 1.12 (d, J=6.9 Hz, 6H)

¹⁹F NMR: (376 MHz, DMSO-d₆) δ −136.23.

### Example 17

### Synthesis of ethyl 2-(2-fluoro-4-(4-hydroxy-3-isopropylbenzyl)-5-(trifluoromethyl)phenoxy)acetate (Compound 17)

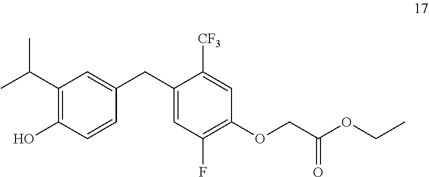 [SMILES: CCOC(=O)COc1cc(C(F)(F)F)c(Cc2ccc(O)c(C(C)C)c2)cc1F]

To a solution of E1′ (100 mg, 318 μmol, 1.0 eq) and 2-isopropylphenol (87 mg, 640 mol, 2.0 eq) in DCE (3 mL) was added ZnCl₂ (1 M, 0.79 mL, 2.5 eq); the mixture was stirred at 85° C. overnight. Water (5 mL) was added, and the mixture was extracted with DCM (5 mL*2). The combined organic layer was washed with brine (10 mL), dried over Na₂SO₄, concentrated to dryness, and purified by Prep-TLC (pet. ether/EtOAc=5/1) to afford Compound 17 (40 mg, 30% yield) as a colorless oil.

¹H NMR: (400 MHz, DMSO-d₆) δ 9.14 (s, 1H), 7.32 (d, J=7.2 Hz, 1H), 7.24 (d, J=5.6 Hz, 1H), 7.02 (d, J=2.2 Hz, 1H), 6.80 (dd, J=8.2, 2.3 Hz, 1H), 6.68 (d, J=8.1 Hz, 1H), 5.00 (s, 2H), 4.16 (q, J=7.1 Hz, 2H), 3.91 (d, J=1.5 Hz, 2H), 3.15 (p, J=6.9 Hz, 1H), 1.18 (t, J=7.1 Hz, 3H), 1.12 (d, J=6.9 Hz, 6H).

### Example 18

### Synthesis of 2-(2-fluoro-4-(4-hydroxy-3-isopropylbenzyl)-5-(trifluoromethyl)phenoxy)acetic acid (Compound 18)

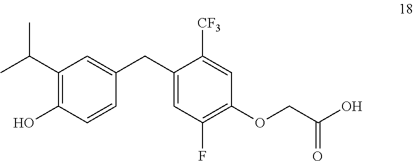 [SMILES: CC(C)c1cc(Cc2cc(F)c(OCC(=O)O)cc2C(F)(F)F)ccc1O]

To a solution of Compound 17 (40 mg, 97 μmol, 1.0 eq) in MeOH/H₂O (2 mL/0.5 mL) at rt was added NaOH (12 mg, 289.58 μmol, 3.0 eq); the mixture was stirred for 1 h at rt. The mixture was diluted with water (10 mL), acidified with 1N HCl to pH=3-4, and extracted with EtOAc (15 mL*2). The combined organic phase was washed with brine (10 mL), dried over Na₂SO₄ and concentrated in vacuo. The crude product was purified by Prep-HPLC to afford Compound 18 (20 mg, 53% yield) as a white solid.

LCMS: T=1.554 min, [M−1]=385.0

¹H NMR: (400 MHz, DMSO-d₆) δ 13.15 (s, 1H), 9.13 (s, 1H), 7.30-7.26 (m, 1H), 7.21 (d, J=5.5 Hz, 1H), 7.02 (d, J=2.2 Hz, 1H), 6.81 (dd, J=8.2, 2.2 Hz, 1H), 6.68 (d, J=8.1 Hz, 1H), 4.89 (s, 2H), 3.91 (s, 2H), 3.18-3.11 (m, 1H), 1.12 (d, J=6.9 Hz, 6H).

### Example 19

### Synthesis of ethyl 2-(3-chloro-4-(2-fluoro-4-hydroxy-3-isopropylbenzyl)phenoxy)acetate (Compound 19)

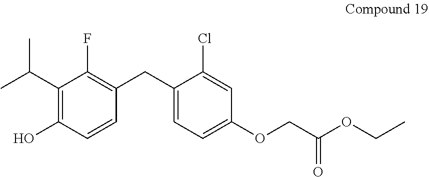 [SMILES: CCOC(=O)COc1ccc(Cc2ccc(O)c(C(C)C)c2F)c(Cl)c1]

To a solution of E2′ (300 mg, 0.97 mmol, 1.0 eq) in DCE (15 mL) at rt were added ZnCl₂ (1M in THF, 3 mL, 3.0 eq) and B2 (451 mg, 2.94 mmol, 3.0 eq). The mixture was heated to 85° C. for 48 h. After cooling, the reaction mixture was diluted with DCM (20 mL), washed with brine (10 mL*2), dried over Na₂SO₄, and concentrated in vacuo. The crude product was purified by Prep-TLC (pet. ether/EtOAc=10/1) to afford Compound 19 (100 mg, 26.9% yield) as a white solid.

TLC: Pet. ether/EtOAc=10/1 (v/v), Rf=0.18

¹H NMR: (400 MHz, DMSO-d₆) δ 9.56 (s, 1H), 7.07 (d, J=8.8 Hz, 1H), 7.03 (d, J=2.4 Hz, 1H), 6.87 (dd, J=8.8, 2.4 Hz, 1H), 6.68 (t, J=8.4 Hz, 1H), 6.53 (d, J=8.4 Hz, 1H), 4.79 (s, 2H), 4.16 (q, J=7.2 Hz, 2H), 3.84 (s, 2H), 1.23 (d, J=7.2 Hz, 6H), 1.19 (d, J=6.8 Hz, 3H).

### Example 20

### Synthesis of 2-(3-chloro-4-(2-fluoro-4-hydroxy-3-isopropylbenzyl)phenoxy)acetic acid (Compound 20)

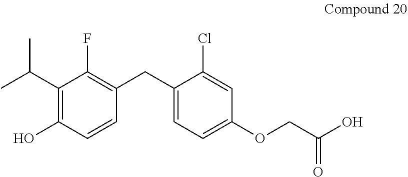 [SMILES: CC(C)c1c(O)ccc(Cc2ccc(OCC(=O)O)cc2Cl)c1F]

To a solution of Compound 19 (100 mg, 0.26 mmol, 1.0 eq) in THF/water (10 mL/1 mL) at rt was added LiOH·H₂O (44 mg, 1.0 mmol, 4.0 eq); the mixture was stirred at rt for 2 h. The mixture was acidified to pH=4-5 with 2N HCl and extracted with EtOAc (20 mL). The organic phase was washed with brine (10 mL), dried over Na₂SO₄, concentrated in vacuo and purified by Prep-HPLC to afford Compound 20 (50 mg, 54% yield) as a white solid.

TLC: DCM/MeOH=5/1 (v/v), Rf=0.28

LCMS: T=1.769 min, [M−1]=351.0

¹H NMR: (400 MHz, DMSO-d₆) δ 12.99 (s, 1H), 9.53 (s, 1H), 7.07 (d, J=8.4 Hz, 1H), 7.00 (d, J=2.4 Hz, 1H), 6.85 (dd, J=8.4, 2.4 Hz, 1H), 6.68 (t, J=8.4 Hz, 1H), 6.54 (d, J=8.0 Hz, 1H), 4.69 (s, 2H), 3.84 (s, 2H), 3.37 (q, J=7.2 Hz, 1H), 1.24 (d, J=6.8 Hz, 6H).

### Example 21

### Synthesis of ethyl 2-(3-bromo-4-(2-fluoro-4-hydroxy-3-isopropylbenzyl)phenoxy)acetate (Compound 21)

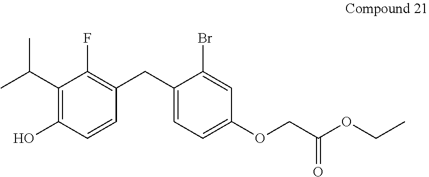 [SMILES: CCOC(=O)COc1ccc(Cc2ccc(O)c(C(C)C)c2F)c(Br)c1]

To a solution of E3′ (4.0 g, 11 mmol, 1.0 eq) in DCE (40 mL) at rt were added B2 (5.3 g, 34 mmol, 3.0 eq) and ZnCl₂ (1 M, 34 mL, 34 mmol, 3.0 eq). The reaction was heated to 90° C. overnight. After cooling, the reaction mixture was diluted with DCM (50 mL), washed with brine (30 mL*2), dried over Na₂SO₄ and concentrated in vacuo. The crude product was purified by silica gel column chromatography (pet. ether/EtOAc=100/1 to 20/1) to afford Compound 21 (1.6 g, 33% yield) as a white solid.

TLC: Pet. ether/EtOAc=5/1 (v/v), Rf=0.33

¹H NMR: (400 MHz, DMSO-d₆) δ 9.55 (d, J=1.6 Hz, 1H), 7.19 (d, J=2.8 Hz, 1H), 7.05 (d, J=8.8 Hz, 1H), 6.91 (dd, J=8.4, 2.4 Hz, 1H), 6.66 (t, J=8.4 Hz, 1H), 6.54 (d, J=8.4 Hz, 1H), 4.79 (s, 2H), 4.16 (q, J=7.2 Hz, 2H), 3.84 (s, 2H), 3.40-3.34 (m, 1H), 1.26-1.18 (m, 9H).

### Example 22

### Synthesis of ethyl 2-(4-(2-fluoro-4-hydroxy-3-isopropylbenzyl)-3-(prop-1-en-2-yl)phenoxy)acetate (Compound 22)

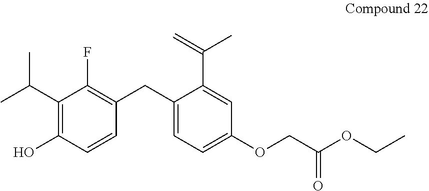 [SMILES: C=C(C)c1cc(OCC(=O)OCC)ccc1Cc1ccc(O)c(C(C)C)c1F]

To a mixture of Compound 21 (300 mg, 0.70 mmol, 1.0 eq) and potassium trifluoro(prop-1-en-2-yl)borate (261 mg, 1.76 mmol, 2.5 eq) in water (1 mL) at rt were added Pd(dppf)Cl₂ (52 mg, 71 μmol, 0.1 eq) and Cs₂CO₃ (460 mg, 1.41 mmol, 2.0 eq) under N₂ (g). The reaction mixture was microwaved at 120° C. for 2 h. After cooling, the mixture was diluted with EtOAc (20 mL), washed with brine (10 mL), dried over Na₂SO₄, and concentrated in vacuo. The residue was purified by silica gel column chromatography (pet. ether/EtOAc=50/1 to 20/1) to afford Compound 22 (220 mg, 80.6% yield) as a white solid.

TLC: Pet. ether/EtOAc=5/1 (v/v), Rf=0.49.

¹H NMR: (400 MHz, DMSO-d₆) δ 9.46 (d, J=1.2 Hz, 1H), 6.94 (d, J=8.8 Hz, 1H), 6.74 (dd, J=8.4, 2.8 Hz, 1H), 6.66 (d, J=2.8 Hz, 1H), 6.59 (t, J=8.4 Hz, 1H), 6.51 (d, J=8.4 Hz, 1H), 5.17 (t, J=2.0 Hz, 1H), 4.75 (dd, J=2.0, 1.2 Hz, 1H), 4.73 (s, 2H), 4.16 (q, J=7.2 Hz, 2H), 3.76 (s, 2H), 3.36 (d, J=7.2 Hz, 1H), 1.90 (d, J=1.2 Hz, 3H), 1.23 (d, J=6.8 Hz, 6H), 1.19 (t, J=7.2 Hz, 3H).

### Example 23

### Synthesis of 2-(4-(2-fluoro-4-hydroxy-3-isopropylbenzyl)-3-(prop-1-en-2-yl)phenoxy)acetic acid (Compound 23)

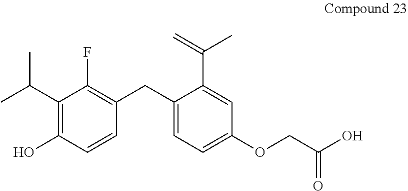 [SMILES: C=C(C)c1cc(OCC(=O)O)ccc1Cc1ccc(O)c(C(C)C)c1F]

To a solution of Compound 22 (80 mg, 0.21 mmol, 1.0 eq) in THF/water (5 mL/1 mL) at rt was added LiOH·H₂O (26 mg, 0.63 mmol, 3.0 eq); the mixture was stirred at rt for 1 h. The reaction was acidified to pH=4-5 with 2N HCl and extracted with EtOAc (5 mL). The organic phase was washed with brine (5 mL), dried over Na₂SO₄ and concentrated in vacuo. The crude material was purified by Prep-HPLC to afford Compound 23 (60 mg, 81% yield) as a white solid.

TLC: DCM/MeOH=5/1 (v/v), Rf=0.37

LCMS: T=3.917 min, [M−1]=357.1

¹H NMR: (400 MHz, DMSO-d₆) δ 9.59 (s, 1H), 6.90 (d, J=8.4 Hz, 1H), 6.67 (dd, J=8.4, 2.8 Hz, 1H), 6.59 (d, J=2.8 Hz, 1H), 6.57 (d, J=8.4 Hz, 1H), 6.51 (d, J=8.4 Hz, 1H), 5.14 (t, J=2.0 Hz, 1H), 4.74 (t, J=1.2 Hz, 1H), 4.38 (s, 2H), 3.74 (s, 2H), 3.38-3.32 (m, 1H), 1.89 (s, 3H), 1.23 (d, J=7.2 Hz, 6H).

### Example 24

### Synthesis of ethyl 2-(4-(2-fluoro-4-hydroxy-3-isopropylbenzyl)-3-isopropylphenoxy)acetate (Compound 24)

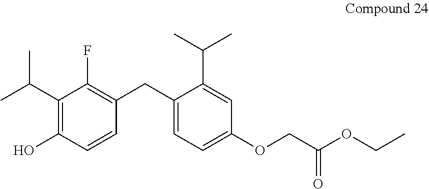 [SMILES: CCOC(=O)COc1ccc(Cc2ccc(O)c(C(C)C)c2F)c(C(C)C)c1]

To a solution of Compound 22 (130 mg, 0.34 mmol) in THF (5 mL) at rt was added Pd/C (41 mg). The mixture was stirred at 50° C. under 1 atm H₂ atmosphere overnight then the reaction was filtered and concentrated in vacuo to afford Compound 24 (130 mg, 99.2% yield) as a colorless oil.

TLC: Pet. ether/EtOAc=5/1 (v/v), Rf=0.51

LCMS: T=2.230 min, [M−1]=387.2.

### Example 25

### Synthesis of 2-(4-(2-fluoro-4-hydroxy-3-isopropylbenzyl)-3-isopropylphenoxy)acetic acid (Compound 25)

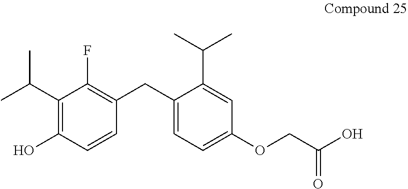 [SMILES: CC(C)c1cc(OCC(=O)O)ccc1Cc1ccc(O)c(C(C)C)c1F]

To a solution of Compound 24 (130 mg, 0.33 mmol, 1.0 eq) in THF/water (5 mL/1 mL) at rt was added LiOH·H₂O (41 mg, 1.0 mmol, 3.0 eq); the mixture was stirred at rt for 1 h. The reaction was acidified to pH=4-5 with 2N HCl and was extracted with EtOAc (5 mL). The organic phase was washed with brine (5 mL), dried over Na₂SO₄, and concentrated in vacuo. The crude material was purified by Prep-HPLC to afford Compound 25 (60 mg, 50% yield) as a white solid.

TLC: Pet. ether/EtOAc=5/1 (v/v), Rf=0.05

LCMS: T=1.058 min, [M−1]=357.0

¹H NMR: (400 MHz, DMSO-d₆) δ 12.94 (s, 1H), 9.47 (d, J=1.2 Hz, 1H), 6.94 (d, J=8.4 Hz, 1H), 6.80 (d, J=2.8 Hz, 1H), 6.63 (dd, J=8.4, 2.8 Hz, 1H), 6.56-6.48 (m, 2H), 4.62 (s, 2H), 3.78 (s, 2H), 3.40 (d, J=7.2 Hz, 1H), 3.08-3.02 (m, 1H), 1.27-1.21 (m, 6H), 1.07 (d, J=6.8 Hz, 6H).

### Example 26

### Synthesis of ethyl 2-(3-chloro-2-fluoro-4-(2-fluoro-4-hydroxy-3-isopropylbenzyl)phenoxy)acetate (Compound 26)

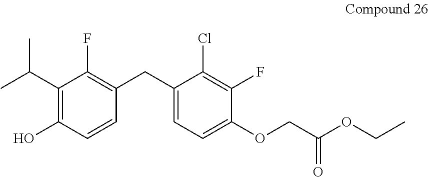 [SMILES: CCOC(=O)COc1ccc(Cc2ccc(O)c(C(C)C)c2F)c(Cl)c1F]

To a solution of E4′ (100 mg, 0.35 mmol, 1.0 eq) in DCE (5 mL) at rt were added 3-fluoro-2-isopropylphenol (B2, 164 mg, 1.07 mmol, 3.0 eq) and ZnCl₂ (710 uL, 0.71 mmol, 2.0 eq). The reaction was heated to 85° C. and stirred overnight. After cooling, the reaction mixture was diluted with DCM (20 mL), washed with brine (20 mL), dried over Na₂SO₄ and concentrated in vacuo. The crude product was purified by Prep-TLC (EtOAc/pet. ether=1/5) to afford Compound 26 (25 mg, 18% yield) as a light yellow oil.

TLC: EtOAc/pet. ether=1/5 (v/v), Rf=0.4

¹H NMR: (400 MHz, DMSO-d₆) δ 9.58 (s, 1H), 7.06 (t, J=8.4 Hz, 1H), 6.93-6.87 (m, 1H), 6.70 (t, J=8.4 Hz, 1H), 6.55 (d, J=8.4 Hz, 1H), 4.89 (s, 2H), 4.16 (q, J=7.6 Hz, 2H), 3.87 (s, 2H), 3.37 (q, J=7.6 Hz, 1H), 1.23 (d, J=7.2 Hz, 6H), 1.21-1.17 (m, 3H).

### Example 27

### Synthesis of 2-(3-chloro-2-fluoro-4-(2-fluoro-4-hydroxy-3-isopropylbenzyl)phenoxy)acetic acid (Compound 27)

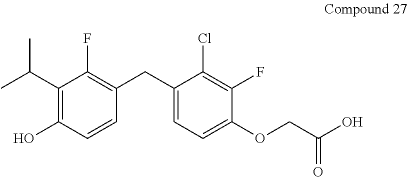 [SMILES: CC(C)c1c(O)ccc(Cc2ccc(OCC(=O)O)c(F)c2Cl)c1F]

To a solution of Compound 26 (70 mg, 180 μmol, 1.0 eq) in THF (3 mL) was added LiOH·H₂O (11 mg, 260 μmol, 1.5 eq) in water (1 mL); the mixture was stirred at rt for 2 h. The reaction was diluted with water (5 mL), acidified with 1N HCl to pH=3-4 and extracted with EtOAc (5 mL*3). The combined organic layers were washed with brine (5 mL), dried over Na₂SO₄ and concentrated in vacuo. The crude product was purified by Prep-TLC (MeOH/DCM=1/10) to afford Compound 27 (20 mg, 31% yield) as an off-white solid.

TLC: DCM/MeOH=10/1 (v/v), Rf=0.2

LCMS: T=3.842 min, [M−1]=369.0

¹H NMR: (400 MHz, DMSO-d₆) δ 9.59 (s, 1H), 7.02 (t, J=8.4 Hz, 1H), 6.90 (d, J=8.4 Hz, 1H), 6.69 (t, J=8.4 Hz, 1H), 6.55 (d, J=8.4 Hz, 1H), 4.74 (s, 2H), 3.86 (s, 2H), 3.23 (m, 1H), 1.23 (d, J=6.8 Hz, 6H)

¹⁹F NMR: (376 MHz, DMSO-d₆) δ −120.14 , −134.51.

### Example 28

### Synthesis of ethyl 2-(5-chloro-2-fluoro-4-(2-fluoro-4-hydroxy-3-isopropylbenzyl)phenoxy)acetate (Compound 28)

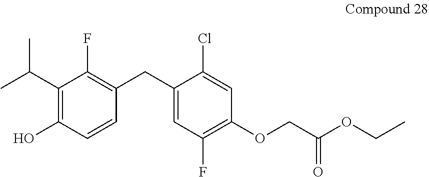 [SMILES: CCOC(=O)COc1cc(Cl)c(Cc2ccc(O)c(C(C)C)c2F)cc1F]

To a solution of E5′ (200 mg, 0.71 mmol, 1.0 eq) in DCE (5 mL) at rt were added ZnCl₂ (1.0 M, 1.42 mL, 1.42 mmol, 2.0 eq) and 3-fluoro-2-isopropylphenol (B2, 329 mg, 2.13 mmol, 3.0 eq). The mixture was heated at reflux and stirred overnight. After cooling to rt, the reaction mixture was diluted with water (5 mL), and extracted with DCM (5 mL*3). The combined organic phase was washed with brine (5 mL), dried over Na₂SO₄ and concentrated in vacuo. The crude product was purified by Prep-TLC (EtOAc/pet. ether=1/5) to afford Compound 28 (70 mg, 25% yield) as a colorless oil.

TLC: EtOAc/pet. ether=1/5 (v/v), Rf=0.4.

### Example 29

### Synthesis of 2-(5-chloro-2-fluoro-4-(2-fluoro-4-hydroxy-3-isopropylbenzyl)phenoxy)acetic acid (Compound 29)

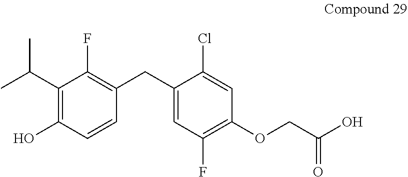 [SMILES: CC(C)c1c(O)ccc(Cc2cc(F)c(OCC(=O)O)cc2Cl)c1F]

To a solution of Compound 28 (70 mg, 180 μmol, 1.0 eq) in THF (5 mL) was added LiOH·H₂O (11 mg, 260 μmol, 1.5 eq) in water (1 mL) and the mixture was stirred at rt for 2 h. The reaction was diluted with water (5 mL), acidified with 1N HCl to pH=3-4, and extracted with EtOAc (3 mL*3). The combined organic layers were washed with brine (5 mL), dried over Na₂SO₄ and concentrated in vacuo. The crude product was purified by Prep-TLC (MeOH/DCM=1/10) to afford Compound 29 (40 mg, 61% yield) as an off-white solid.

TLC: DCM/MeOH=10/1 (v/v), Rf=0.2

LCMS: T=3.786 min, [M−1]=369.0

¹H NMR: (400 MHz, DMSO-d₆) δ 9.62 (s, 1H), 7.12 (d, J=8.0 Hz, 1H), 7.01 (d, J=12.0 Hz, 1H), 6.69 (t, J=8.4 Hz, 1H), 6.55 (d, J=8.4 Hz, 1H), 4.64 (s, 2H), 3.82 (s, 2H), 3.39 (d, J=6.8 Hz, 1H), 1.24 (d, J=6.8 Hz, 6H).

¹⁹F NMR: (376 MHz, DMSO-d₆) δ −120.11 , −136.07.

### Example 30

### Synthesis of ethyl 2-(2-fluoro-4-(2-fluoro-4-hydroxy-3-isopropylbenzyl)-3-isopropylphenoxy)acetate (Compound 30)

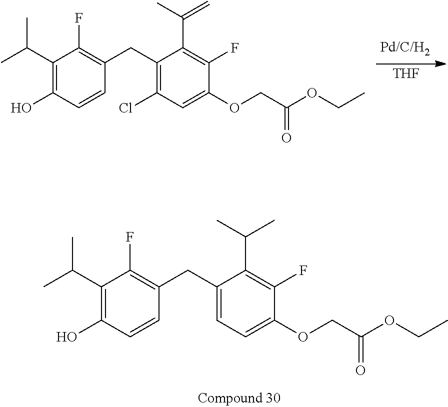

To a solution of Intermediate F2′ (140 mg, 319 μmol, 1.0 eq) in THF (2.0 mL) was added Pd/C (70 mg). The mixture was flushed with H₂ three times and stirred under 1 atm H₂ atmosphere at 60° C. overnight. The mixture was filtered and concentrated in vacuo to afford Compound 30 (60 mg, 43% yield) as a yellow oil.

TLC: Pet. ether/EtOAc=1/5 (v/v), Rf=0.18.

### Example 31

### Synthesis of 2-(2-fluoro-4-(2-fluoro-4-hydroxy-3-isopropylbenzyl)-3-isopropylphenoxy)acetic acid (Compound 31)

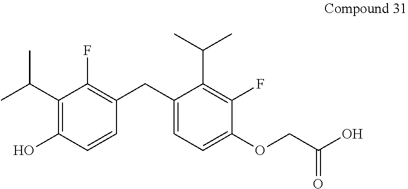 [SMILES: CC(C)c1c(O)ccc(Cc2ccc(OCC(=O)O)c(F)c2C(C)C)c1F]

To a solution of Compound 30 (60 mg, 150 μmol, 1.0 eq) in THF/H₂O (2 mL/0.5 mL) at rt was added LiOH H₂O (19 mg, 440 μmol, 3.0 eq); the mixture was stirred at rt for 1 h. The mixture was diluted with water (30 mL), acidified to pH=3-4 with 1 N HCl, and extracted with EtOAc (15 mL*3). The combined organic layers were washed with brine (30 mL), dried over Na₂SO₄, and concentrated in vacuo. The crude material was purified by Prep-HPLC to afford Compound 31 (15 mg, 27% yield) as a white solid.

TLC: MeOH/DCM=1/10 (v/v), Rf=0.20

LCMS: T=1.955 min, [M−1]=376

¹H NMR: (400 MHz, DMSO-d₆) δ 9.49 (s, 1H), 6.85-6.77 (m, 2H), 6.53-6.47 (m, 2H), 4.69 (s, 2H), 3.80 (s, 2H), 3.08 (p, J=7.2 Hz, 1H), 1.27-1.22 (m, 6H), 1.15 (dd, J=7.2, 1.2 Hz, 6H)

¹⁹F NMR: (376 MHz, DMSO-d₆) δ −120.48 , −136.36.

### Example 32

### Synthesis of ethyl 2-(5-bromo-2-fluoro-4-(4-hydroxy-3-isopropylbenzyl)phenoxy)acetate (Compound 32)

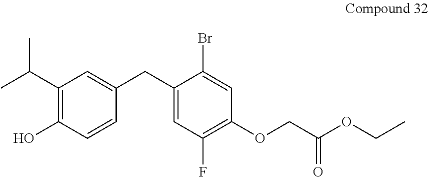 [SMILES: CCOC(=O)COc1cc(Br)c(Cc2ccc(O)c(C(C)C)c2)cc1F]

To a solution of Compound E6′ (200 mg, 0.61 mmol, 1.0 eq) in DCE (5 mL) at rt were added 2-isopropylphenol (167 mg, 1.23 mmol, 2.0 eq) and ZnCl₂ (209 mg, 1.54 mmol, 2.5 eq). The reaction was heated to 90° C. and stirred overnight. After cooling, the reaction mixture was diluted with DCM (20 mL), washed with brine (10 mL*2), dried over Na₂SO₄ and concentrated in vacuo. The crude product was purified by Prep-TLC (pet. ether/EtOAc=5/1) to afford Compound 32 (140 mg, 53.6% yield) as a white solid.

TLC: Pet. ether/EtOAc=5/1 (v/v), Rf=0.29

LCMS: T=1.479 min, [M−1]=422.9

¹H NMR: (400 MHz, DMSO-d₆) δ 9.11 (s, 1H), 7.37 (d, J=8.0 Hz, 1H), 7.15 (d, J=12.0 Hz, 1H), 7.00 (d, J=2.4 Hz, 1H), 6.77 (dd, J=8.0, 2.0 Hz, 1H), 6.67 (d, J=8.0 Hz, 1H), 4.91 (s, 2H), 4.17 (q, J=7.2 Hz, 2H), 3.84 (s, 2H), 3.15 (p, J=6.8 Hz, 1H), 1.20 (t, J=7.2 Hz, 3H), 1.12 (d, J=6.8 Hz, 6H).

### Example 33

### Synthesis of ethyl 2-(2-fluoro-4-(4-hydroxy-3-isopropylbenzyl)-5-(prop-1-en-2-yl)phenoxy)acetate (Compound 33)

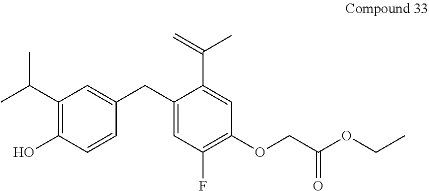 [SMILES: C=C(C)c1cc(OCC(=O)OCC)c(F)cc1Cc1ccc(O)c(C(C)C)c1]

To a mixture of Compound 32 (140 mg, 0.32 mmol, 1.0 eq) and potassium trifluoro(prop-1-en-2-yl)borate (97 mg, 0.66 mmol, 2.0 eq) in water (1 mL) at rt were added Pd(dppf)Cl₂ (27 mg, 32.9 μmol, 0.1 eq) and Cs₂CO₃ (214 mg, 0.66 mmol, 2.0 eq) under N₂ (g). The reaction mixture was microwaved at 120° C. for 2 h. After cooling, the mixture was diluted with EtOAc (20 mL), washed with brine (10 mL), dried over Na₂SO₄ and concentrated in vacuo. The residue was purified by Prep-TLC (pet. ether/EtOAc=5/1) to afford Compound 33 (110 mg, 86.6% yield) as a colorless oil.

TLC: Pet. ether/EtOAc=5/1 (v/v), Rf=0.49.

LCMS: T=1.568 min, [M−1]=385.0.

¹H NMR: (400 MHz, DMSO-d₆) δ 9.05 (s, 1H), 6.92 (d, J=12.8 Hz, 1H), 6.87 (d, J=2.0 Hz, 1H), 6.80 (d, J=8.8 Hz, 1H), 6.69 (dd, J=8.0, 2.4 Hz, 1H), 6.66 (d, J=8.0 Hz, 1H), 5.20 (t, J=2.0 Hz, 1H), 4.84 (s, 2H), 4.78 (s, 1H), 4.16 (q, J=7.2 Hz, 2H), 3.77 (s, 2H), 3.14 (p, J=6.8 Hz, 1H), 1.89 (s, 3H), 1.20 (t, J=9.2 Hz, 3H), 1.10 (d, J=6.8 Hz, 6H).

### Example 34

### Synthesis of 2-(2-fluoro-4-(4-hydroxy-3-isopropylbenzyl)-5-(prop-1-en-2-yl)phenoxy)acetic acid (Compound 34)

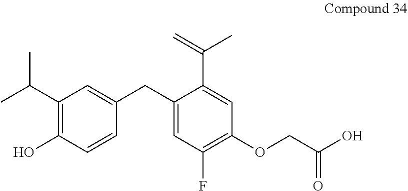 [SMILES: C=C(C)c1cc(OCC(=O)O)c(F)cc1Cc1ccc(O)c(C(C)C)c1]

To a solution of Compound 33 (110 mg, 0.28 mmol, 1.0 eq) in MeOH/water (5 mL/1 mL) at rt was added LiOH H₂O (36 mg, 0.85 mmol, 3.0 eq). The mixture was stirred at rt for 1 h, then water (10 mL) was added. The reaction was acidified to pH=3-4 with 1N HCl and extracted with EtOAc (10 mL*2). The combined organic phase was washed with brine (20 mL), dried over Na₂SO₄ and concentrated in vacuo to afford Compound 34 (75 mg, 74% yield) as a yellow oil.

TLC: Pet. ether/EtOAc=5/1 (v/v), Rf=0.04.

LCMS: T=1.058 min, [M−1]=357.0.

### Example 35

### Synthesis of 2-(2-fluoro-4-(4-hydroxy-3-isopropylbenzyl)-5-isopropylphenoxy)acetic acid (Compound 35)

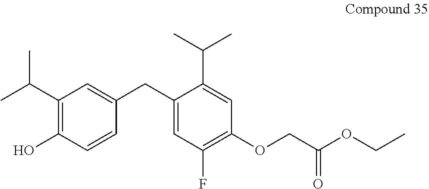 [SMILES: CCOC(=O)COc1cc(C(C)C)c(Cc2ccc(O)c(C(C)C)c2)cc1F]

To a solution of Compound 33 in THE at rt is added Pd/C (10%, 50% by weight). The mixture is flushed with H₂ three times and stirred under 1 atm of H₂ at 60° C. overnight. The mixture is filtered and concentrated in vacuo to afford Compound 35.

### Example 36

### Synthesis of 2-(2-fluoro-4-(4-hydroxy-3-isopropylbenzyl)-5-isopropylphenoxy)acetic acid (Compound 36)

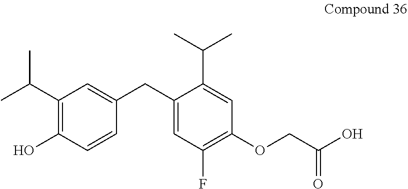 [SMILES: CC(C)c1cc(Cc2cc(F)c(OCC(=O)O)cc2C(C)C)ccc1O]

To a solution of Compound 34 (75 mg, 0.21 mmol) in THF (5 mL) at rt was added Pd/C (15 mg). The mixture was stirred at 60° C. under 1 atm H₂ overnight, then the mixture was filtered, concentrated in vacuo, and purified by Prep-HPLC to afford Compound 36 (40 mg, 53% yield) as a white solid.

TLC: DCM/MeOH=5/1 (v/v), Rf=0.35

LCMS: T=1.616 min, [M−1]=359.1

¹H NMR: (400 MHz, DMSO-d₆) δ 13.02 (s, 1H), 9.04 (s, 1H), 6.92 (d, J=3.2 Hz, 1H), 6.89 (s, 1H), 6.87 (d, J=1.6 Hz, 1H), 6.68 (dd, J=8.4, 2.0 Hz, 1H), 6.65 (d, J=8.4 Hz, 1H), 4.74 (s, 2H), 3.80 (s, 2H), 3.17-3.06 (m, 2H), 1.10 (d, J=6.8 Hz, 6H), 1.04 (d, J=6.8 Hz, 6H).

### Example 37

### Synthesis of ethyl 2-(3-chloro-2,6-difluoro-4-(4-hydroxy-3-isopropylbenzyl)phenoxy)acetate (Compound 37)

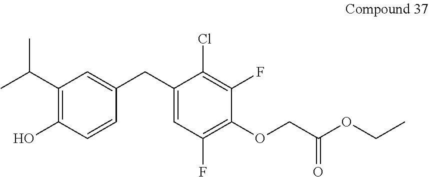 [SMILES: CCOC(=O)COc1c(F)cc(Cc2ccc(O)c(C(C)C)c2)c(Cl)c1F]

To a solution of E7′ (200 mg, 0.67 mmol, 1.0 eq) and 2-isopropylphenol (273 mg, 2.01 mmol, 3.0 eq) in DCE (3 mL) was added ZnCl₂ (1 Min THF, 1.67 mL, 1.67 mmol, 2.5 eq). The mixture was stirred at 85° C. overnight, then concentrated to dryness in vacuo. Water (5 mL) was added, and the mixture was extracted with DCM (5 mL*2). The combined organic layers were washed with brine (10 mL), dried over Na₂SO₄, concentrated to dryness and purified by Prep-TLC (pet. ether/EtOAc=5/1) to afford Compound 37 (30 mg, 11% yield) as a colorless oil.

¹H NMR: (400 MHz, DMSO-d₆) δ 9.14 (s, 1H), 7.10 (dd, J=12.2, 2.2 Hz, 1H), 7.01 (d, J=2.2 Hz, 1H), 6.78 (dd, J=8.2, 2.3 Hz, 1H), 6.68 (d, J=8.2 Hz, 1H), 4.86 (s, 2H), 4.14 (q, J=7.1 Hz, 2H), 3.90 (s, 2H), 3.15 (p, J=6.9 Hz, 1H), 1.17 (d, J=7.1 Hz, 3H), 1.12 (d, J=6.9 Hz, 6H).

### Example 38

### Synthesis of 2-(3-chloro-2,6-difluoro-4-(4-hydroxy-3-isopropylbenzyl)phenoxy)acetic acid (Compound 38)

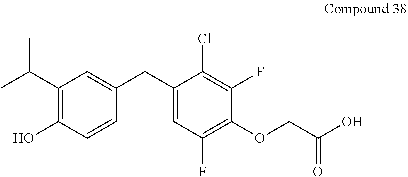 [SMILES: CC(C)c1cc(Cc2cc(F)c(OCC(=O)O)c(F)c2Cl)ccc1O]

To a solution of Compound 37 (30 mg, 75 μmol, 1.0 eq) in MeOH/H₂O (2 mL/0.5 mL) at rt was added NaOH (9.0 mg, 230 μmol, 3.0 eq); the mixture was stirred at rt for 1 h. The mixture was diluted with water (10 mL), acidified to pH=3-4 with 1N HCl, and extracted with EtOAc (15 mL*2). The combined organic layers were washed with brine (10 mL), dried over Na₂SO₄ and concentrated in vacuo. The crude product was purified by Prep-HPLC to afford Compound 38 (10 mg, 35% yield) as a white solid.

LCMS: T=2.309 min, [M+Na]=392.9

¹H NMR: (400 MHz, DMSO-d₆) δ 13.00 (s, 1H), 9.14 (s, 1H), 7.08 (dd, J=12.3, 2.2 Hz, 1H), 7.01 (d, J=2.2 Hz, 1H), 6.78 (dd, J=8.2, 2.2 Hz, 1H), 6.68 (d, J=8.1 Hz, 1H), 4.78 (s, 2H), 3.89 (s, 2H), 3.14 (q, J=6.9 Hz, 1H), 1.12 (d, J=6.9 Hz, 6H).

### Example 39

### Synthesis of 2-(5-chloro-2-fluoro-4-(2-fluoro-4-hydroxy-3-isopropylbenzyl)phenoxy)-N-methylacetamide (Compound 39)

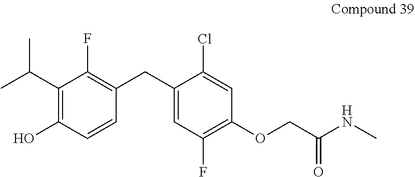 [SMILES: CNC(=O)COc1cc(Cl)c(Cc2ccc(O)c(C(C)C)c2F)cc1F]

Compound 39 was prepared starting from Compound 29 using the method described in Example 11.

LCMS: [M−1]=382.2

### Example 40

### Synthesis of 2-(5-chloro-2-fluoro-4-(2-fluoro-4-hydroxy-3-isopropylbenzyl)phenoxy)-N,N-dimethylacetamide (Compound 40)

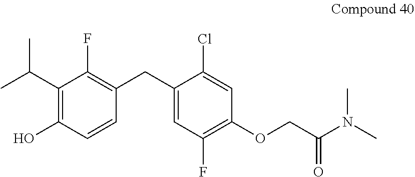 [SMILES: CC(C)c1c(O)ccc(Cc2cc(F)c(OCC(=O)N(C)C)cc2Cl)c1F]

Compound 40 was prepared starting from Compound 29 using the method described in Example 12.

LCMS: [M−1]=396.1

### Example 41

### Synthesis of 2-(4-(2-fluoro-4-hydroxy-3-isopropylbenzyl)-3-(prop-1-en-2-yl)phenoxy)-N-methylacetamide (Compound 41)

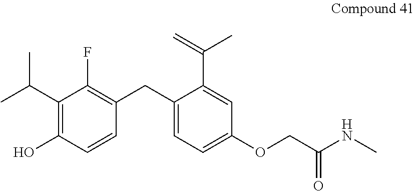 [SMILES: C=C(C)c1cc(OCC(=O)NC)ccc1Cc1ccc(O)c(C(C)C)c1F]

Compound 41 was prepared starting from Compound 23 using the method described in Example 11.

LCMS: [M+1]=372.2

### Example 42

### Synthesis of 2-(4-(2-fluoro-4-hydroxy-3-isopropylbenzyl)-3-(prop-1-en-2-yl)phenoxy)-N,N-dimethylacetamide (Compound 42)

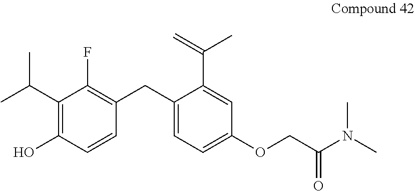 [SMILES: C=C(C)c1cc(OCC(=O)N(C)C)ccc1Cc1ccc(O)c(C(C)C)c1F]

Compound 42 was prepared starting from Compound 23 using the method described in Example 12.

LCMS: [M−1]=384.2

### Example 43

### Synthesis of 2-(4-(2-fluoro-4-hydroxy-3-isopropylbenzyl)-3-isopropylphenoxy)-N-methylacetamide (Compound 43)

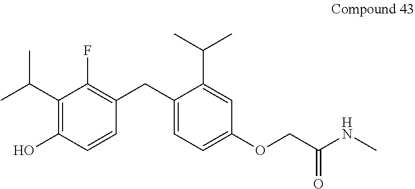 [SMILES: CNC(=O)COc1ccc(Cc2ccc(O)c(C(C)C)c2F)c(C(C)C)c1]

Compound 43 was prepared starting from Compound 25 using the method described in Example 11.

LCMS: [M+1]=374.1

### Example 44

### Synthesis of 2-(4-(2-fluoro-4-hydroxy-3-isopropylbenzyl)-3-isopropylphenoxy)-N,N-dimethylacetamide (Compound 44)

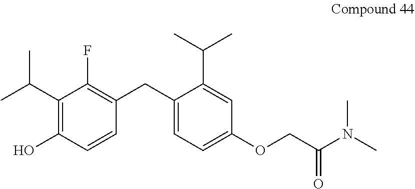 [SMILES: CC(C)c1cc(OCC(=O)N(C)C)ccc1Cc1ccc(O)c(C(C)C)c1F]

Compound 44 was prepared starting from Compound 25 using the method described in Example 12.

LCMS: [M+1]=388.1

### Example 45

### Synthesis of 2-(2-fluoro-4-(4-hydroxy-3-isopropylbenzyl)-5-(prop-1-en-2-yl)phenoxy)-N-methylacetamide (Compound 45)

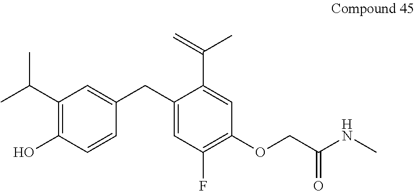 [SMILES: C=C(C)c1cc(OCC(=O)NC)c(F)cc1Cc1ccc(O)c(C(C)C)c1]

Compound 45 was prepared starting from Compound 33 using the method described in Example 11.

LCMS: [M−1]=372.1

### Example 46

### Synthesis of 2-(2-fluoro-4-(4-hydroxy-3-isopropylbenzyl)-5-(prop-1-en-2-yl)phenoxy)-N,N-dimethylacetamide (Compound 46)

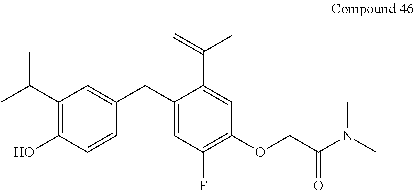 [SMILES: C=C(C)c1cc(OCC(=O)N(C)C)c(F)cc1Cc1ccc(O)c(C(C)C)c1]

Compound 46 was prepared starting from Compound 33 using the method described in Example 12.

LCMS: [M−1]=384.2

### Example 47

### Synthesis of 2-(2-fluoro-4-(4-hydroxy-3-isopropylbenzyl)-5-isopropylphenoxy)-N-methylacetamide (Compound 47)

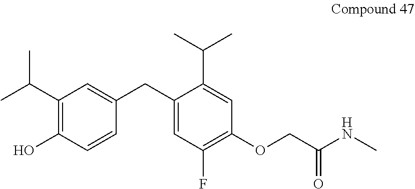 [SMILES: CNC(=O)COc1cc(C(C)C)c(Cc2ccc(O)c(C(C)C)c2)cc1F]

Compound 47 was prepared starting from Compound 36 using the method described in Example 11.

LCMS: [M−1]=372.2

### Example 48

### Synthesis of 2-(2-fluoro-4-(4-hydroxy-3-isopropylbenzyl)-5-isopropylphenoxy)-N,N-dimethylacetamide (Compound 48)

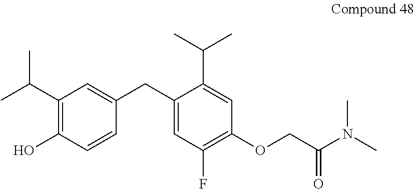 [SMILES: CC(C)c1cc(Cc2cc(F)c(OCC(=O)N(C)C)cc2C(C)C)ccc1O]

Compound 48 was prepared starting from Compound 36 using the method described in Example 12.

LCMS: [M−1]=386.2

### Example 49

### Biological Assays

### Thyroid-Hormone Reporter-Gene Assays

Compounds were tested for thyroid-hormone receptor activity using TR reporter-gene assays. Reporter cells used in the assays express a TR-receptor hybrid (either TRα or TRO) in which the native N-terminal DNA binding domain (DBD) has been replaced with that of the yeast Gal4 DBD. The reporter gene, firefly luciferase, is functionally linked to the Gal4 upstream activation sequence (UAS). Both cell lines were derived from human embryonic kidney (HEK293).

Step 1: A suspension of reporter cells was prepared in cell recovery medium containing 10% charcoal-stripped FBS, and dispensed into assay plates. The plates were pre-incubated for 6 hours in a cell culture incubator (37° C./5% CO2/85% humidity).

Step 2: Test compound master stocks and triiodothyronine were diluted in DMSO to generate solutions at “1,000×-concentration” relative to each final treatment concentration. These intermediate stocks were subsequently diluted directly into compound screening medium containing 10% charcoal-stripped FBS to generate “2×-concentration” treatment media (containing 0.2, 0.4 or 0.8% DMSO).

Step 3: At the end of the pre-incubation period, culture media were discarded from the assay plates, and all wells received 100 μl of compound screening medium. 100 μl of each of the previously prepared “2×-concentration” treatment media were dispensed into duplicate assay wells, thereby achieving the desired final treatment concentrations. The final concentration of DMSO in all assay wells was 0.1, 0.2 or 0.4%. Assay plates were incubated for 24 hr in a cell culture incubator (37° C./5% CO2/85% humidity).

Step 4: At the 24 h assay endpoint, treatment media were discarded and 100 l/well of luciferase detection reagent was added. Relative luminometer units (RLUs) were quantified from each assay well. The performance of the TRα and TRβ assays was validated using the reference agonist triiodothyronine (T3).

The results of these assays are presented in Table 3 below, wherein data are reported as EC₅₀ values determined for TRα and TRβ receptors, and the selectivity index (SI) is calculated as EC₅₀ (TRα)/EC₅₀ (TRβ). To this end, EC₅₀ and SI values are expressed as follows:
Potency: +EC₅₀>1,000 nM
   ++100 nM<EC₅₀≤1,000 nM
   +++10 nM<EC₅₀≤100 nM
   ++++EC₅₀≤10 nM
   ND Not determined
Selectivity: +T3−SI≤3X
   ++3X<T3−SI≤30X
   +++T3−SI>30X
   ND Not determined

TABLE 3
Activity Data

| Compound | | | |
|---|---|---|---|
| No. | TRα | TRβ | T3-SI |
| T3 | ++++ | +++ | + |
| 2 | + | ++ | ++ |
| 4 | + | + | ND |
| 6 | ++ | +++ | ++ |
| 8 | +++ | ++++ | ++ |
| 10 | +++ | ++++ | ++ |
| 14 | + | ++ | ++ |
| 16 | + | + | ++ |
| 18 | + | + | + |
| 20 | + | ++ | ++ |
| 23 | + | ++ | ++ |
| 25 | ++ | +++ | ++ |
| 27 | + | + | ND |
| 29 | + | ++ | +++ |
| 31 | + | + | ++ |
| 36 | ++ | ++ | ++ |
| 38 | ND | + | ND |

As indicated by the above experiments, compounds of the present invention show improved TRβ selectivity when compared to the natural agonist T3 as may also show improved potency when compared to T3.

### FAAH Substrate Evaluation

Purified recombinant human FAAH (rhFAAH) was purchased from Cayman Chemical (Ann Arbor, Mich., USA). The total volume for each incubation was 400 μL containing a final 0.5 ng/μL rhFAAH, 1 μM test compound, 1.25% ethanol or 1 μM PF-3845 (FAAH inhibitor), and 0.1% bovine serum albumin in Tris-EDTA buffer at pH 8.0). The positive control was LL-341001. The incubation was conducted at the room temperature. At 0, 5, 15, 30 and 60 minutes, an aliquot of 30 μL reaction mixtures was removed and mixed with 300 μL acetonitrile containing 5 ng/mL terfenadine and 10 ng/mL tolbutamide as internal standards to quench the reaction. The resulting mixture was centrifuged at 4000 rpm, 4° C. for 15 minutes, and 100 μL supernatant was ready for LC-MS/MS analysis to measure the formation of acid metabolite.

### LC-MS/MS Analysis

Acquity Ultra Performance LC system from Waters was used for sample analysis. The chromatography was performed on a reverse phase Kinetex 2.6 μm C18 column, 2.1×30 mm, 100 Å. The mobile phase A comprised of 0.10% formic acid in water and mobile phase B comprised of 0.1% formic acid in acetonitrile with a 2-min run time at the flow rate of 0.8 mL/min for the acid metabolite from positive control or a 1.5 min run time at the flow rate of 0.9 mL/min for the acid metabolite of test compounds. The mass spectrometer (API-5500 and API Q Trap 4000 Applied Biosystems/MDS SCIEX Instruments, Framingham, Mass., USA) was operated under ESI positive or negative ion MRM mode.

### Data Analysis

The formation of acid metabolite was monitored and quantified using one calibration point of 1 μM. The observed rate constant (ke) for the acid metabolite formation was calculated by plotting the metabolite concentration versus time of incubation with the slope being ke and is shown in Table 4.

TABLE 4

| Compound | ke | Compound | ke | Compound | ke |
|---|---|---|---|---|---|
| 11 | A | 12 | ND | 39 | A |
| 40 | ND | 41 | A | 42 | ND |
| 43 | A | 44 | ND | 45 | A |
| 46 | ND | 47 | A | 48 | A |

A = ke is less than 2.0 and more than or equal to 0.05;
ND = not determined.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the structure of Formula (I): 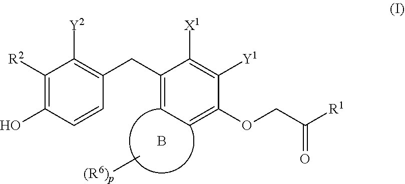
or a pharmaceutically acceptable isomer, racemate, tautomer, hydrate, solvate, isotope, or salt thereof, wherein:
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
R² is lower alkyl, lower alkenyl, carbocycle, heterocycle, carbocyclealkyl, or heterocyclealkyl;
each R⁶ is, independently, lower alkyl, lower alkenyl, lower haloalkyl, or halo; and
p is 0-5;
wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

2. The compound of claim 1, or a pharmaceutically acceptable isomer, racemate, tautomer, hydrate, solvate, isotope, or salt thereof, wherein B is carbocycle.

3. The compound of claim 1 or claim 2, or a pharmaceutically acceptable isomer, racemate, tautomer, hydrate, solvate, isotope, or salt thereof, wherein B is a 5-6 membered carbocycle.

4. The compound of any one of claims 1-3, or a pharmaceutically acceptable isomer, racemate, tautomer, hydrate, solvate, isotope, or salt thereof, wherein B is a 5-6 membered cycloalkyl.

5. The compound of any one of claims 1-3, or a pharmaceutically acceptable isomer, racemate, tautomer, hydrate, solvate, isotope, or salt thereof, wherein B is a 5-6 membered aryl.

6. The compound of claim 1, or a pharmaceutically acceptable isomer, racemate, tautomer, hydrate, solvate, isotope, or salt thereof, wherein B is heterocycle.

7. The compound of claim 1 or claim 6, or a pharmaceutically acceptable isomer, racemate, tautomer, hydrate, solvate, isotope, or salt thereof, B is a 5-6 membered heterocycle.

8. The compound of any one of claim 1 or 6-7, or a pharmaceutically acceptable isomer, racemate, tautomer, hydrate, solvate, isotope, or salt thereof, wherein B is a 5-6 membered heterocycloalkyl.

9. The compound of any one of claim 1 or 6-7, or a pharmaceutically acceptable isomer, racemate, tautomer, hydrate, solvate, isotope, or salt thereof, wherein B is a 5-6 membered heteroaryl.

10. The compound of any one of claims 1-9, or a pharmaceutically acceptable isomer, racemate, tautomer, hydrate, solvate, isotope, or salt thereof, wherein R² is lower alkyl optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

11. The compound of any one of claims 1-10, or a pharmaceutically acceptable isomer, racemate, tautomer, hydrate, solvate, isotope, or salt thereof, wherein R² is unsubstituted lower alkyl.

12. The compound of any one of claims 1-11, having the structure of Formula (II): 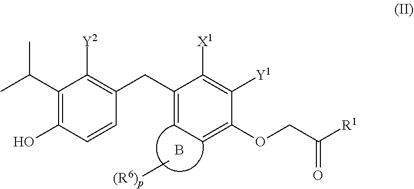
or a pharmaceutically acceptable isomer, racemate, tautomer, hydrate, solvate, isotope, or salt thereof, wherein:
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle; and
R^{1c} is H, lower alkyl, carbocycle, heterocycle, carbocyclealkyl, or heterocyclealkyl;
each R⁶ is, independently, lower alkyl, lower alkenyl, lower haloalkyl, or halo; and
p is 0-5;
wherein R^{1a}, R^{1b}, and R^{1c} are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

13. The compound of claim 1, wherein R² is carbocyclealkyl or heterocyclealkyl and having the structure of Formula (III): 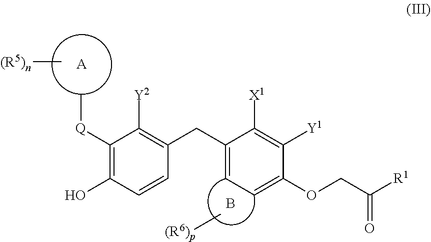
or a pharmaceutically acceptable isomer, racemate, tautomer, hydrate, solvate, isotope, or salt thereof, wherein:
A is aryl or heteroaryl;
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
Q is —C(R³R⁴)— or —{C(R³R⁴)}₂—;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle; and
R^{1c} is H, lower alkyl, carbocycle, heterocycle, carbocyclealkyl, or heterocyclealkyl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocycle alkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
R^{a} and R^{b} are each, independently, H, lower alkyl, or lower haloalkyl;
each R⁶ is, independently, lower alkyl, lower alkenyl, lower haloalkyl, or halo;
n is 0-5; and
p is 0-5;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

14. The compound of claim 1, having the structure of Formula (IV): 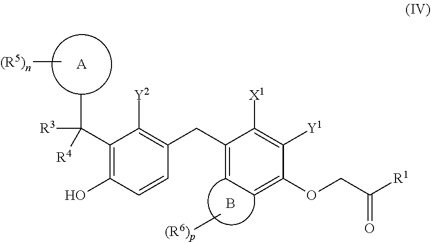
or a pharmaceutically acceptable isomer, racemate, tautomer, hydrate, solvate, isotope, or salt thereof, wherein:
A is aryl or heteroaryl;
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
R^{a} and R^{b} are each, independently, H, lower alkyl, or lower haloalkyl;
each R⁶ is, independently, lower alkyl, lower alkenyl, lower haloalkyl, or halo;
n is 0-5; and
p is 0-5;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

15. The compound of claim 1, having the structure of Formula (V): 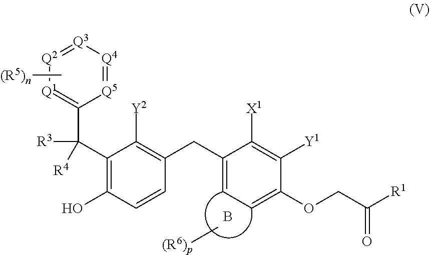
or a pharmaceutically acceptable isomer, racemate, tautomer, hydrate, solvate, isotope, or salt thereof, wherein:
Q¹, Q², Q³, Q⁴, and Q⁵ are each, independently, CH, CR⁵, or N;
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
R^{a} and R^{b} are each, independently, H, lower alkyl, or lower haloalkyl;
each R⁶ is, independently, lower alkyl, lower alkenyl, lower haloalkyl, or halo;
n is 0-5; and
p is 0-5;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

16. The compound of claim 1, having the structure of Formula (VI): 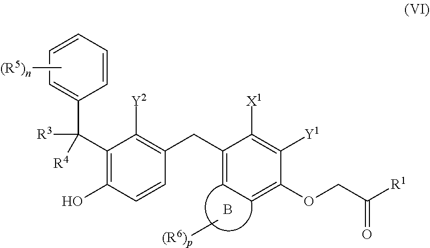
or a pharmaceutically acceptable isomer, racemate, tautomer, hydrate, solvate, isotope, or salt thereof, wherein:
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
R^{a} and R^{b} are each, independently, H, lower alkyl, or lower haloalkyl;
each R⁶ is, independently, lower alkyl, lower alkenyl, lower haloalkyl, or halo;
n is 0-5; and
p is 0-5;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

17. The compound of claim 1, having the structure of Formula (VII): 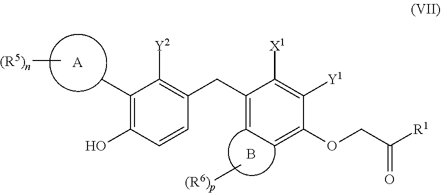
or a pharmaceutically acceptable isomer, racemate, tautomer, hydrate, solvate, isotope, or salt thereof, wherein:
A is aryl or heteroaryl;
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
R^{a} and R^{b} are each, independently, H, lower alkyl, or lower haloalkyl;
each R⁶ is, independently, lower alkyl, lower alkenyl, lower haloalkyl, or halo;
n is 0-5; and
p is 0-5;
wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

18. The compound of claim 1, having the structure of Formula (VIII): 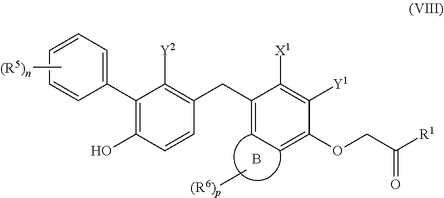
or a pharmaceutically acceptable isomer, racemate, tautomer, hydrate, solvate, isotope, or salt thereof, wherein:
B is a carbocyle or a heterocyle;
X¹ is H, lower alkyl, lower alkenyl, lower haloalkyl, or halo;
Y¹ is H, —CN, halo, lower alkyl, or lower alkoxy;
Y² is H, —CN, halo, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
R^{a} and R^{b} are each, independently, H, lower alkyl, or lower haloalkyl;
each R⁶ is, independently, lower alkyl, lower alkenyl, lower haloalkyl, or halo;
n is 0-5; and
p is 0-5;
wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

19. The compound of any one of claims 13-16, or a pharmaceutically acceptable isomer, racemate, tautomer, hydrate, solvate, isotope, or salt thereof, wherein R³ is H or lower alkyl.

20. The compound of any one of claims 13-16, or a pharmaceutically acceptable isomer, racemate, tautomer, hydrate, solvate, isotope, or salt thereof, wherein R³ is methyl, ethyl, or propyl.

21. The compound of any one of claims 1-20, or a pharmaceutically acceptable isomer, racemate, tautomer, hydrate, solvate, isotope, or salt thereof, wherein R¹ is —NR^{1a}R^{1b}.

22. The compound of any one of claims 1-21, or a pharmaceutically acceptable isomer, racemate, tautomer, hydrate, solvate, isotope, or salt thereof, wherein R^{1a} is lower alkyl.

23. The compound of claim 22, or a pharmaceutically acceptable isomer, racemate, tautomer, hydrate, solvate, isotope, or salt thereof, wherein R^{1a} is methyl.

24. The compound of any one of claims 1-23, or a pharmaceutically acceptable isomer, racemate, tautomer, hydrate, solvate, isotope, or salt thereof, wherein R^{1b} is H.

25. The compound of any one of claims 1-20, or a pharmaceutically acceptable isomer, racemate, tautomer, hydrate, solvate, isotope, or salt thereof, wherein R¹ is —OR^{1c}.

26. The compound of claim 25, or a pharmaceutically acceptable isomer, racemate, tautomer, hydrate, solvate, isotope, or salt thereof, wherein R^{1c} is H.

27. The compound of claim 25, or a pharmaceutically acceptable isomer, racemate, tautomer, hydrate, solvate, isotope, or salt thereof, wherein R^{1c} is lower alkyl.

28. The compound of any one of claims 1-27, or a pharmaceutically acceptable isomer, racemate, tautomer, hydrate, solvate, isotope, or salt thereof, wherein X¹ is H.

29. The compound of any one of claims 1-27, or a pharmaceutically acceptable isomer, racemate, tautomer, hydrate, solvate, isotope, or salt thereof, wherein X¹ is lower alkyl.

30. The compound of claim 29, or a pharmaceutically acceptable isomer, racemate, tautomer, hydrate, solvate, isotope, or salt thereof, wherein X¹ is methyl.

31. The compound of any one of claims 1-30, or a pharmaceutically acceptable isomer, racemate, tautomer, hydrate, solvate, isotope, or salt thereof, wherein Y¹ is halo.

32. The compound of any one of claims 1-30, or a pharmaceutically acceptable isomer, racemate, tautomer, hydrate, solvate, isotope, or salt thereof, wherein Y¹ is H.

33. The compound of any one of claims 1-32, or a pharmaceutically acceptable isomer, racemate, tautomer, hydrate, solvate, isotope, or salt thereof, wherein Y² is halo.

34. The compound of any one of claims 1-32, or a pharmaceutically acceptable isomer, racemate, tautomer, hydrate, solvate, isotope, or salt thereof, wherein Y² is H.

35. The compound of any one of claims 1-34, or a pharmaceutically acceptable isomer, racemate, tautomer, hydrate, solvate, isotope, or salt thereof, wherein p is 0-3.

36. The compound of any one of claims 1-35, or a pharmaceutically acceptable isomer, racemate, tautomer, hydrate, solvate, isotope, or salt thereof, wherein at least one R⁶ is lower alkyl.

37. The compound of any one of claims 1-35, or a pharmaceutically acceptable isomer, racemate, tautomer, hydrate, solvate, isotope, or salt thereof, wherein p is 0.

38. A compound having the structure of any one of the compounds listed in Table 1, or a pharmaceutically acceptable isomer, racemate, hydrate, solvate, isotope, or salt thereof, having the structure of any one of the compounds listed in Table 1.

39. A compound having the structure of Formula (I′): 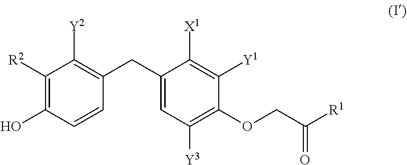
or a pharmaceutically acceptable isomer, racemate, tautomer, hydrate, solvate, isotope, or salt thereof, wherein:
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl; and
R² is lower alkyl, lower alkenyl, carbocycle, heterocycle, carbocyclealkyl, or heterocyclealkyl;
wherein R^{1a}, R^{1b}, R^{1c}, and R² are each, independently, optionally substituted with one or more halo, lower alkyl, lower haloalkyl, —CN, —OR′, —NR′R″, ═O, ═S, —S(O)₂R′ or —S(O)₂OR′, wherein R′ and R″ are each, independently, H, lower alkyl, or lower haloalkyl.

40. The compound of claim 39, or a pharmaceutically acceptable isomer, racemate, tautomer, hydrate, solvate, isotope, or salt thereof, wherein R² is lower alkyl optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

41. The compound of claim 39 or claim 40, or a pharmaceutically acceptable isomer, racemate, tautomer, hydrate, solvate, isotope, or salt thereof, wherein R² is unsubstituted lower alkyl.

42. The compound of claim 39, having the structure of Formula (II′): 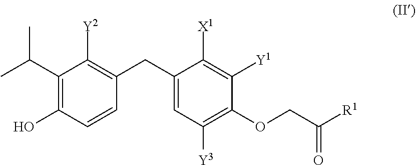
or a pharmaceutically acceptable isomer, racemate, tautomer, hydrate, solvate, isotope, or salt thereof, wherein:
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl; and
wherein R^{1a}, R^{1b}, and R^{1c} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

43. The compound of claim 39, wherein R² is carbocyclealkyl or heterocyclealkyl and having the structure of Formula (III′): 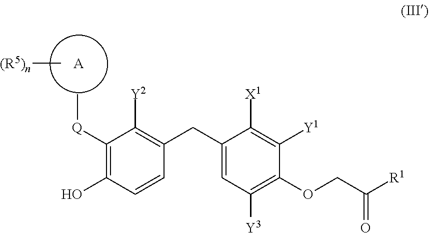
or a pharmaceutically acceptable isomer, racemate, tautomer, hydrate, solvate, isotope, or salt thereof, wherein:
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
Q is —C(R³R⁴)— or —{C(R³R⁴)}₂—;
A is aryl or heteroaryl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
n is 0-5; and
R^{a} and R^{b} are each, independently, H, lower alkyl, or lower haloalkyl;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

44. The compound of claim 39 having the structure of Formula (IV′): 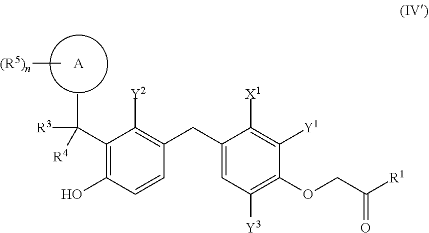
or a pharmaceutically acceptable isomer, racemate, tautomer, hydrate, solvate, isotope, or salt thereof, wherein:
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
A is aryl or heteroaryl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
n is 0-5; and
R^{a} and R^{b} are each, independently, H, lower alkyl, or lower haloalkyl;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

45. The compound of claim 39 having the structure of Formula (V′): 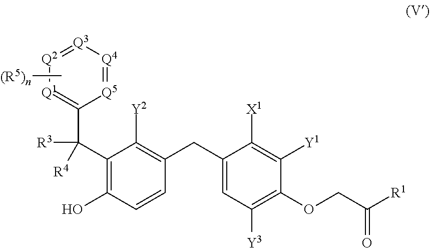
or a pharmaceutically acceptable isomer, racemate, tautomer, hydrate, solvate, isotope, or salt thereof, wherein:
Q¹, Q², Q³, Q⁴, and Q⁵ are each, independently, CH, CR⁵, or N;
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
n is 0-5; and
R^{a} and R^{b} are each, independently, H, lower alkyl, or lower haloalkyl;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

46. The compound of claim 39 having the structure of Formula (VI′): 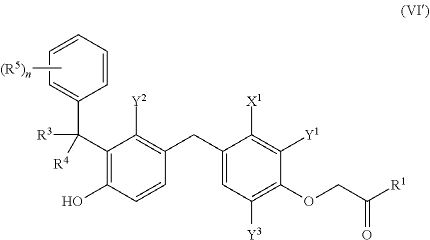
or a pharmaceutically acceptable isomer, racemate, tautomer, hydrate, solvate, isotope, or salt thereof, wherein:
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
R³ and R⁴ are each, independently, H, halo, —CN, lower alkyl, lower alkenyl, lower alkynyl, lower haloalkyl, —OR^{a}, —NR^{a}R^{b}, carbocycle, heterocycle, carbocyclealkyl, or heterocyclealkyl, or R³ and R⁴, together, form ═O or ═S;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
n is 0-5; and
R^{a} and R^{b} are each, independently, H, lower alkyl, or lower haloalkyl;
wherein R^{1a}, R^{1b}, R^{1c}, R³, R⁴, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

47. The compound of claim 39 having the structure of Formula (VII′): 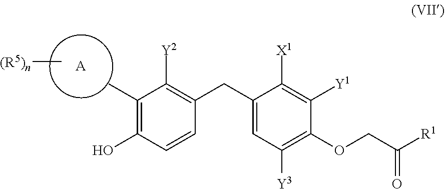
or a pharmaceutically acceptable isomer, racemate, tautomer, hydrate, solvate, isotope, or salt thereof, wherein:
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
A is aryl or heteroaryl;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
n is 0-5; and
R^{a} and R^{b} are each, independently, H, lower alkyl, or lower haloalkyl;
wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

48. The compound of claim 39 having the structure of Formula (VIII′): 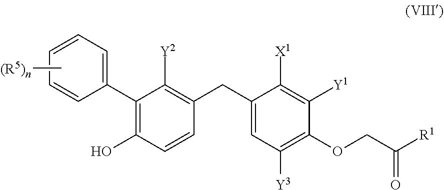
or a pharmaceutically acceptable isomer, racemate, tautomer, hydrate, solvate, isotope, or salt thereof, wherein:
X¹ is lower alkyl, lower alkenyl, lower haloalkyl, or halo;
Y¹ is H, —CN, halogen, lower alkyl, or lower alkoxy;
Y² is H or halogen;
Y³ is H, —CN, halogen, lower alkyl, or lower alkoxy;
R¹ is —NR^{1a}R^{1b} or —OR^{1c};
R^{1a} and R^{1b} are each, independently, H, lower alkyl, lower alkenyl, lower alkynyl, —OR^{a}, —NR^{a}R^{b}, carbocycle, carbocyclealkyl, heterocycle, or heterocyclealkyl, or R^{1a} and R^{1b} taken together with the nitrogen atom to which they are attached form heterocycle;
R^{1c} is H, lower alkyl, carbocycle, heterocycle, carbocyclealkyl, or heterocyclealkyl;
each R⁵ is, independently, halo, —CN, lower alkyl, lower alkenyl, lower alkynyl, lower haloalkyl, carbocycle, heterocycle, carbocyclealkyl, heterocyclealkyl, —OR^{a}, —NR^{a}R^{b}, —C(O)R^{a}, —C(O)OR^{a}, —C(O)NR^{a}R^{b}, —NR^{a}C(O)R^{b}, —S(O)₂R^{a}, or —S(O)₂OR^{a};
n is 0-5; and
R^{a} and R^{b} are each, independently, H, lower alkyl, or lower haloalkyl;
wherein R^{1a}, R^{1b}, R^{1c}, R⁵, R^{a}, and R^{b} are each, independently, optionally substituted with one or more halo, —CN, —OR′, —NR′R″, ═O, ═S, —S(O)₂R′ or —S(O)₂OR′, wherein R′ and R″ are each, independently, H, lower alkyl, or lower haloalkyl.

49. The compound of any one of claims 43-46, or a pharmaceutically acceptable isomer, racemate, tautomer, hydrate, solvate, isotope, or salt thereof, wherein R³ is H.

50. The compound of any one of claims 39-49, or a pharmaceutically acceptable isomer, racemate, tautomer, hydrate, solvate, isotope, or salt thereof, wherein R¹ is —NR^{1a}R^{1b}.

51. The compound of any one of claims 39-50, or a pharmaceutically acceptable isomer, racemate, tautomer, hydrate, solvate, isotope, or salt thereof, wherein R^{1a} is lower alkyl.

52. The compound of claim 51, or a pharmaceutically acceptable isomer, racemate, tautomer, hydrate, solvate, isotope, or salt thereof, wherein R^{1a} is methyl.

53. The compound of any one of claims 39-52, or a pharmaceutically acceptable isomer, racemate, tautomer, hydrate, solvate, isotope, or salt thereof, wherein R^{1b} is H.

54. The compound of any one of claims 39-49, or a pharmaceutically acceptable isomer, racemate, tautomer, hydrate, solvate, isotope, or salt thereof, wherein R¹ is —OR^{1c}.

55. The compound of claim 54, or a pharmaceutically acceptable isomer, racemate, tautomer, hydrate, solvate, isotope, or salt thereof, wherein R^{1c} is H.

56. The compound of claim 54, or a pharmaceutically acceptable isomer, racemate, tautomer, hydrate, solvate, isotope, or salt thereof, wherein R^{1c} is lower alkyl.

57. The compound of any one of claims 39-56, or a pharmaceutically acceptable isomer, racemate, tautomer, hydrate, solvate, isotope, or salt thereof, wherein X¹ is lower alkyl.

58. The compound of any one of claims 39-56, or a pharmaceutically acceptable isomer, racemate, tautomer, hydrate, solvate, isotope, or salt thereof, wherein X¹ is halo.

59. The compound of any one of claims 39-56, or a pharmaceutically acceptable isomer, racemate, tautomer, hydrate, solvate, isotope, or salt thereof, wherein X¹ is lower haloalkyl.

60. The compound of any one of claims 39-56, or a pharmaceutically acceptable isomer, racemate, tautomer, hydrate, solvate, isotope, or salt thereof, wherein X¹ is lower alkenyl.

61. The compound of any one of claims 39-60, or a pharmaceutically acceptable isomer, racemate, tautomer, hydrate, solvate, isotope, or salt thereof, wherein Y¹ is halogen.

62. The compound of any one of claims 39-60, or a pharmaceutically acceptable isomer, racemate, tautomer, hydrate, solvate, isotope, or salt thereof, wherein Y¹ is H.

63. The compound of any one of claims 39-62, or a pharmaceutically acceptable isomer, racemate, tautomer, hydrate, solvate, isotope, or salt thereof, wherein Y² is halogen.

64. The compound of any one of claims 39-62, or a pharmaceutically acceptable isomer, racemate, tautomer, hydrate, solvate, isotope, or salt thereof, wherein Y² is H.

65. The compound of any one of claims 39-64, or a pharmaceutically acceptable isomer, racemate, tautomer, hydrate, solvate, isotope, or salt thereof, wherein Y³ is halogen.

66. The compound of any one of claims 39-64, or a pharmaceutically acceptable isomer, racemate, tautomer, hydrate, solvate, isotope, or salt thereof, wherein Y³ is H.

67. A compound having the structure of any one of the compounds listed in Table 2, or a pharmaceutically acceptable isomer, racemate, tautomer, hydrate, solvate, isotope, or salt thereof.

68. A pharmaceutical composition comprising a compound of any one of claims 1-67, or a pharmaceutically acceptable isomer, racemate, hydrate, solvate, isotope, or salt thereof, and a pharmaceutically acceptable excipient.

69. A method of treating a subject having a neurodegenerative disease comprising administering to the subject in need thereof a pharmaceutically effective amount of the compound of any one of claims 1-67, or a pharmaceutically salt or composition thereof.

70. The method of claim 69, wherein the neurodegenerative disease is a demyelinating disease.

71. The method of claim 69 or 70, wherein the neurodegenerative disease is adult Refsum disease, Alexander disease, Alzheimer's disease, Balo concentric sclerosis, Canavan disease, central pontine myelinolysis, cerebral palsy, cerebrotendineous xanthomatosis, chronic inflammatory demyelinating polyneuropathy, Devic's syndrome, diffuse myelinoclastic sclerosis, idiopathic inflammatory demyelinating disease, infantile Refsum disease, Krabbe disease, Leber hereditary optic neuropathy, Marburg multiple sclerosis, Marchiafava-Bignami disease, metachromatic leukodystrophy, multifocal motor neuropathy, paraproteinemic demyelinating polyneuropathy, Pelizaeus-Merzbacher disease, peroneal muscular atrophy, progressive multifocal leukoencephalopathy, transverse myelitis, tropical spastic paraparesis, van der Knaap disease, X-linked adrenoleukodystrophy, or Zellweger syndrome.
